(19) United States
(12) Patent Application Publication
(10) Pub. No.: US 2025/0180508 A1
(43) Pub. Date: Jun. 5, 2025

(54) QUANTITATIVE LIPIDOMIC ANALYSIS, METHODS AND USES THEREOF

(71) Applicant: McMaster University, Hamilton (CA)

(72) Inventors: Philip Britz-McKibbin, Hamilton (CA); Ritchie Ly, Stoney Creek (CA)

(21) Appl. No.: 18/965,513

(22) Filed: Dec. 2, 2024

Related U.S. Application Data
(60) Provisional application No. 63/604,948, filed on Dec. 1, 2023.
(60) Provisional application No. 63/604,953, filed on Dec. 1, 2023.

(51) Int. Cl. G01N 27/447; G01N 1/40; G01N 33/92

(52) U.S. Cl. CPC G01N 27/44704; G01N 1/4055; G01N 33/92; G01N 2405/06; G01N 2560/00; G01N 2570/00; G01N 2800/32; G01N 2800/50; G01N 2800/52

(57) ABSTRACT

This disclosure relates to the field of lipidomics, and in particular, to a novel derivatization strategy for quantitative lipid methylation and uses thereof. Further, this disclosure relates to use of phospholipid biomarkers for assessing and monitoring Omega-3 Index (O3I), including methods and uses thereof.

### RELATED APPLICATIONS

This application claims benefit of U.S. Provisional Patent Application Ser. No. 63/604,948 filed Dec. 1, 2023, and U.S. Provisional Patent Application Ser. No. 63/604,953 filed Dec. 1, 2023, both of which are incorporated herein by reference in their entirety.

### FIELD

The present disclosure relates to the field of lipidomics, and in particular, to a novel derivatization strategy for quantitative lipid methylation. The present disclosure further relates to phospholipid biomarkers for assessing Omega-3 Index status in a subject and uses thereof.

### BACKGROUND

The human lipidome comprises a vast number of lipid molecular species present in tissues, cells, exosomes and biofluids, which are defined by their specific polar head group, chemical linkage, fatty acid carbon chain length, number of double bond equivalents, oxygenated fatty acyls, and regio-/stereochemistry.^{1,2} As lipid homeostasis plays an important role in energy metabolism, membrane structure, and cell signalling, dysregulation in lipid metabolism has long been associated with inflammation and the etiology of cardiometabolic disorders, including obesity, type 2 diabetes, cardiovascular and neurodegenerative diseases.^{3,4} Lipidomic studies have also gained traction in nutritional epidemiology as objective indicators of food exposures since essential dietary fats and fat-soluble vitamins relevant to human health⁵ are not accurately assessed from self-reports.⁶ For these reasons, new advances in untargeted lipid profiling by high resolution mass spectrometry (MS)⁷ provide a hypothesis-generating approach for gaining new insights into complex disease mechanisms.⁸ However, several technical hurdles impede the progress in lipidomics given the lack of chemical standards and reference MS/MS spectra that limit comparative quantitative reporting and the unambiguous identification of unknown lipids of clinical significance.⁹ Recent efforts have focused on developing consensus guidelines in lipid classification and annotation,^{10,11} using internal standards for data normalization,¹² applying automated data processing with open-access software tools,^{13,14} as well as implementing standardized lipidomic protocols and inter-laboratory ring trials using reference and quality control samples.¹⁵⁻¹⁷ Nevertheless, lipidomics workflows require careful method optimization to avoid bias and false discoveries depending on the specific biospecimen type and instrumental platform, including sample pretreatment protocols.¹⁸

Nutritional epidemiological studies have relied on food frequency questionnaires to estimate omega-3 FA dietary fat intake for chronic disease risk assessment (71). Alternatively, biomarkers may offer a more reliable way to assess nutritional status (72) given between-subject differences in n3-LCPUFA bioavailability and metabolism, the variable content of omega-3 FAs in marine foods, and memory recall bias. In this case, the omega-3 index (O3I), defined as the erythrocyte EPA+DHA content from the phospholipid (PL) fraction as a mole percent to total fatty acids, represents a novel biomarker of coronary heart disease risk and sudden cardiac death independent of traditional risk factors (20). Although PL erythrocytes reflect habitual n3-LCPUFA intake patterns over a longer time interval (˜120 days) as compared to other more dynamic PL class pools in circulation (77), a moderate to strong correlation of the O3I with EPA and DHA PL content measured in plasma or whole dried blood has been reported previously (74,78). The disadvantages of the existing O3I index include the need to access erythrocytes, which are not widely available in biorepositories unlike other blood specimens (79,80). Also, gas chromatography (GC) requires complicated sample handling procedures for O3I status determination after off-line PL fractionation by thin layer chromatography and their subsequent saponification into FA methyl esters, which is time consuming and less amenable to large-scale epidemiological studies (81). Also, there is a lack of standardization when reporting the O3I as varying number of FAs are measured by GC methods complicating comparative studies. Therefore, there is currently a need for improved methods to assess O3I that are more amenable to routine screening.

The background herein is included solely to explain the context of the disclosure. This is not to be taken as an admission that any of the material referred to was published, known, or part of the common general knowledge as of the priority date.

### SUMMARY

Herein, a novel two-step chemical derivatization strategy is introduced for the quantitative methylation of PLs based on 9-fluorenylmethyoxycarbonyl chloride (FMOC) followed by 3-methyl-1-p-tolyltriazene (MTT) that offers a practical way to expand lipidome coverage in mass spectrometry, such as MSI-NACE-MS. For the first time, it is demonstrated that this procedure enables the rapid identification and quantification of phosphatidylcholines (PCs) and sphingomyelins (SMs) as their cationic phosphate methyl esters, which was validated on a standard reference human plasma sample previously analyzed in an inter-laboratory harmonization study.¹⁵

This two-stage FMOC/MTT lipid methylation derivatization strategy can also be applied to improve the resolution and detection of other classes of lipids when using complementary liquid chromatography-mass spectrometry, direct infusion-mass spectrometry and ion mobility-mass spectrometry methods.

An accelerated data workflow using a sub-group analysis of serum extracts from placebo and high-dose fish oil (FO) treatment participants confirmed that dietary omega-3 fatty acids were predominately uptaken as their phosphatidylcholines (PCs) in comparison to other serum phospholipid pools. Consistently in both FO (5.0 g/day) and docosahexaenoic acid (DHA) or eicosapentaenoic acid (EPA)-specific (3.0 g/day) intervention studies, serum PC (16:0_20:5) was most responsive (>7-fold change from baseline) to supplementation (>28 days) as compared to various DHA containing PCs, notably PC (16:0_22:6) (>2-fold change from baseline), reflective of preferential incorporation of EPA into these circulating lipid pools. It was also demonstrated that the sum of serum PC (16:0_20:5) and PC (16:0_22:6) was positively correlated to 031 measurements when using FO (r=0.717, p=1.62×10⁻¹¹, n=69), as well as DHA or EPA (r=0.764, p=3.00×10⁻³³, n=167) with most participants improving their O3I status >8.0%. However, DHA was more efficacious in improving O3I (ΔO3I=4.90+1.33) compared to EPA (ΔO3I=2.99+1.19) that was dose dependent with large between-subject variability. It was concluded that MSI-NACE-MS offers a promising multiplexed separation platform for more convenient assessment of O3I status using specific PCs derived from widely available serum or plasma specimens.

Other instrumental methods can also be used for rapid screening of the 031 status based on these circulating PLs with or without chemical labeling, including direct infusion-tandem mass spectrometry, liquid chromatography-mass spectrometry, and ion mobility-mass spectrometry. This work can support nutritional epidemiological studies exploring the role of essential dietary fats in human health while optimizing individual responses to dietary or pharmacological interventions based on specific omega-3 fatty acid formulations. Accordingly, the present disclosure describes the identification of circulating phospholipid species that may serve as surrogate biomarkers of O3I status using the novel derivatization strategy described herein.

Accordingly, an aspect of the disclosure is a method of generating a lipid profile using mass spectrometry (MS), the method comprising:
a) extracting a lipid fraction from a sample obtained from a subject;
b) protecting the amine of one or more lipids in the lipid fraction using an amine protecting reagent to provide one or more protected lipids;
c) methylating a phosphate ester to generate a phosphate methyl ester of the one or more protected lipids using 3-methyl-1-p-tolyltriazene (MTT) to generate one or more methylated lipids;
d) optionally back extracting the one or more methylated lipids;
e) optionally separating the one or more methylated lipids;
f) introducing the one or more methylated lipids to a mass spectrometer under positive-ion mode; and
g) acquiring a mass spectrum chart of the one or more methylated lipids to generate the lipid profile of the sample.

In some embodiments, the amine protecting reagent is 9-fluorenylmethyoxycarbonyl chloride (FMOC).

In some embodiments, separating, in step e) comprises capillary electrophoresis, liquid chromatography or ion mobility.

In some embodiments, extracting the lipid fraction in step a) comprises incubating the sample with methyl tert butyl ether (MTBE), hexane, chloroform, methanol or acetonitrile.

In some embodiments, extracting the lipid fraction in step a) comprises incubating the sample with methyl tert butyl ether (MTBE).

In some embodiments, step b) further comprises drying the one or more protected lipids under nitrogen.

In some embodiments, the one or more lipids is a phospholipid, glycerolipid, glycerophospholipid, sphingolipid, sterol, steroid, isoprenoid, glycolipid, polyketide, saccharolipid, prenol lipid, bile acid, fatty acid, a lipid containing a reactive amino, carboxyl, phenol, thiol, hydroxyl, or phosphate functionality (e.g., acylcarnitines, acyl-coenzyme A) or combinations thereof.

In some embodiments, the one or more lipids is a phospholipid.

In some embodiments, the phospholipid is sphingomyelin (SM), phosphatidylcholine (PC), phosphatidylserine (PS), phosphatidylinositol (PI), phosphatidylethanolamine (PE), phosphatidic acid (PA), lysophosphatidylcholine (LPC), lysophosphatidylethanolamine (LPE), cardiolipin (CL) lysophosphatidic acid (LPA), or combinations thereof.

In some embodiments, the method further comprises in step e) introducing a known amount of one or more methylated lipids as a reference sample for calibration of lipid quantity.

In some embodiments, the one or more lipids in the lipid fraction in step b) are cationic or zwitterionic. In some embodiments, the one or more methylated lipids are cationic or zwitterionic, optionally the one or more methylated lipids are zwitterionic or cationic phospholipids.

In some embodiments, step c) has a reaction time of about 20 minutes to about 100 minutes, about 20 minutes to about 40 minutes, about 40 minutes to about 60 minutes, about 60 minutes to about 100 minutes, about 60 minutes to about 80 minutes, or about 40 minutes to about 80 minutes, optionally the reaction time is about 60 minutes.

In some embodiments, the concentration of MTT in step c) is about 50 mM to about 900 mM, optionally, the MTT is at a concentration of about 450 mM.

In some embodiments, step c) has a reaction temperature of about 20° C. to about 100° C., optionally the reaction temperature is about 60° C.

In some embodiments, step b) has a reaction time of about 1 minute to about 30 minutes.

In some embodiments, step b) has a reaction time of about 5 minutes.

In some embodiments, the concentration of the amine protecting reagent in step b) is about 0.10 mM to about 10 mM.

In some embodiments, the concentration of the amine protecting reagent in step b) is about 0.85 mM.

In some embodiments, in step f) the mass spectrometer comprises multisegment injection-nonaqueous capillary electrophoresis-mass spectrometry (MSI-NACE-MS), direct infusion-MS, desorption ionization (DESI)-MS, gas chromatography (GC)-MS, ion mobility (IM)-MS liquid chromatography (LC)-MS or supercritical fluid chromatography (SFC)-MS (SFC)-MS/MS.

In some embodiments, in step f) the mass spectrometer is multisegment injection-nonaqueous capillary electrophoresis-mass spectrometry (MSI-NACE-MS).

In some embodiments, the sample is of animal, plant or human origin.

In some embodiments, the sample is from human blood, optionally plasma or serum.

In some embodiments, in step b) the amine protecting reagent is added in excess and the excess amine protecting reagent reacts with p-toluidine and/or phosphatidylethanolamine (PE).

In some embodiments, back extracting the one or more methylated lipids in step d) comprises back extracting with hexane.

In some embodiments, back extracting the one or more methylated lipids in step d) comprises back extracting with methyl tert butyl ether (MTBE).

In some embodiments, the lipid profile is untargeted.

In some embodiments, the lipid profile is targeted.

In some embodiments, the method further comprises separating a portion of the lipid fraction obtained in step a) for mass spectrometry in negative ion mode.

In some embodiments, negative ion mode is for detecting anionic lipids and a mass spectrum chart is generated and results are combined with the lipid profile of the sample in g).

Another aspect of the disclosure is a method of assessing omega-3 index (O3I) status in a subject, the method comprising:
a) obtaining a first sample from the subject at a first time point;
b) measuring the level of one or more omega-3 containing phospholipid biomarkers in the first sample; and
c) comparing the level of the one or more omega-3 containing phospholipid biomarkers in the first sample to a control level or value of the one or more omega-3 containing phospholipid biomarkers of known O3I status, wherein similarity of level of the phospholipid biomarkers in the sample with the control indicates that the sample is from a subject with the known O3I status.

In some embodiments, the one or more omega-3 containing phospholipid biomarkers comprises one or more phosphatidylcholines (PCs) selected from the group consisting of PC 38:6 (16:0_22:6), PC 36:5 (16:0_20:5), PC 38:5, PC 40:6, PC 36:6, and PC 40:5.

In some embodiments, the one or more PCs comprise one PC, two PCs, three PCs, four PCs, five PCs or six PCs.

In some embodiments, the one or more PCs comprise PC 36:5 (16:0_20:5) and PC 38:6 (16:0_22:6).

In some embodiments, the one or more omega-3 containing phospholipid biomarkers consist of two PCs, and wherein the two PCs consist of PC 36:5 (16:0_20:5) and PC 38:6 (16:0_22:6).

In some embodiments, the one or more omega-3 containing phospholipid biomarkers comprise PCs comprising omega-3 fatty acids containing eicosapentaenoic acid (EPA, 20:5). docosahexaenoic acid (DHA, 22:6), docosapentaenoic acid (DPA) and/or alpha-linolenic acid (ALA) together with other fatty acyl chains (e.g., 18:0). In some embodiments, the PCs comprise EPA and/or DHA.

In some embodiments, the sample comprises serum, plasma, whole blood or dried blood.

In some embodiments, the method further comprises repeating the method of assessing O3I status for a second sample taken from the same subject at a second time point to assess O3I status and monitor change from the first time point to the second time point.

In some embodiments, assessing the level of one or more omega-3 containing phospholipid biomarkers comprises a method of chemical derivatization, the method comprising:
a) protecting the amine of the one or more omega-3 containing phospholipid biomarkers using an amine protecting reagent to provide one or more protected lipids, optionally the amine protecting reagent is FMOC;
b) methylating a phosphate ester of the one or more protected omega-3 containing phospholipid biomarkers using 3-methyl-1-p-tolyltriazene (MTT) to generate one or more methylated omega-3 containing phospholipid biomarkers.

In some embodiments, the method further comprises introducing the one or more methylated lipids to a mass spectrometer under positive-ion mode and acquiring a mass spectrum chart of the one or more methylated lipids to generate the lipid profile of the sample.

Another aspect of the disclosure is a method of assessing cardiovascular risk in a subject, the method comprising: assessing omega-3 index (O3I) status in a subject using the methods described herein,

wherein if the level of the one or more omega-3 containing phospholipid biomarkers is similar to a control of less than 4% O3I the subject is determined to be at high cardiovascular risk, if the level of the one or more omega-3 containing phospholipid biomarkers is similar to a control of 4% to 8% O3I the subject is determined to be at intermediate cardiovascular risk, and if the level of the one or more omega-3 containing phospholipid biomarkers is similar to a control of more than 8% O3I the subject is determined to be at low cardiovascular risk.

In some embodiments, if the subject has a high or intermediate cardiovascular risk, the method further comprises treating the subject by administering omega-3 fatty acid supplementation.

In some embodiments, the omega-3 fatty acid supplementation comprises fish oil, eicosapentaenoic acid (EPA) and/or docosahexaenoic acid (DHA).

The preceding section is provided by way of example only and is not intended to be limiting on the scope of the present disclosure and appended claims. Additional objects and advantages associated with the compositions and methods of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional advantages objects and embodiments are expressly included within the scope of the present disclosure. The publications and other materials used herein to illuminate the background of the disclosure, and in particular cases, to provide additional details respecting the practice, are incorporated by reference, and for convenience are listed in the appended reference section.

### DRAWINGS

Further objects, features and advantages of the disclosure will become apparent from the following detailed description taken in conjunction with the accompanying figures showing illustrative embodiments of the disclosure, in which:

FIGS. 1A-B show FMOC/MTT derivatization. FIG. 1A depicts an overview of FMOC/MTT derivatization scheme proposed as a safer alternative to hazardous diazomethane to render zwitter-ionic PLs with a net positive charge as their methylphosphate esters. The initial addition of excess FMOC reacts with interfering PEs to avoid isobaric interferences with PCs following methylation while also reacting with p-toluidine as major by-product in the reaction to reduce ion suppression prior to hexane back extraction. FIG. 1B shows series of extracted ion electropherograms in MSI-NACE-MS under positive ion mode that highlight the large mobility shift following methylation, where methylated (cationic) phospholipids (PC) migrate faster than the EOF (top) with improved resolution and separation efficiency. Major ion suppression for the reference mass is evident for native zwitter-ionic PCs co-migrating close to the EOF, which is avoided after their chemical derivatization.

FIG. 2 shows the proposed mechanism for methylation of phospholipids using MTT in an exemplary embodiment of the disclosure. Briefly, this reaction involves a proton transfer to amino group of MTT for activation to increase its electrophilic character resulting in formation of p-tolyltriazene and a methylated phosphate ester head group with a net cationic charge on the phospholipid. This reaction subsequently liberates N2 gas with concomitant generation of p-toluidine as a major by-product.

FIG. 3 shows the impact of the FMOC/MTT derivatization The top panel shows a reaction scheme depicting p-toluidine by-product generation after methylation of phospholipids from MTT. The bottom panel shows prior to the introduction of FMOC, a singly charged molecular ion [M+H]⁺ associated with formation of p-toluidine (m/z 108.081) was observed within the migration time (MT) separation window for methylated PC species. FMOC not only was required to react with PEs to prevent interference with isobaric PC species, but also to react with p-toluidine and form a neutral adduct, thereby preventing ion suppression as excess p-toluidine otherwise migrated close to methylated phospholipids in MSI-NACE-MS.

FIG. 4 shows an extracted ion electropherogram overlay of purine (m/z 121.0509) used as reference mass calibrant in the sheath liquid solution under different sample workup conditions following FMOC/MTT derivatization of NIST SRM-1950 plasma extracts by MSI-NACE-MS under positive ion mode detection in an exemplary embodiment of the disclosure. Two major regions of ion suppression correspond to excess MTT by-product (e.g., p-toluidine) and the EOF, where abundant neutral lipid classes co-migrate (e.g., cholesterol esters, diacylglycerides). Two different organic solvents (i.e., MTBE vs. hexane) were used for back extraction of methylated phospholipids following chemical derivatization as a way to reduce ion suppression effects as compared to a standard run without washing (bottom trace). Notably, the use of hexane (top trace) outperformed MTBE (middle trace) resulting in a superior sample cleanup that greatly reduced ion suppression from excess reagents in a region where methylated phospholipids migrate when using multiplexed separations by MSI-NACE-MS.

FIGS. 5A,B show MS/MS spectra acquired after CID experiments on derivatized methylated SM 34:1; O2 in positive and negative ion mode when using NACE-MS (single injection) in an exemplary embodiment of the disclosure. FIG. 5A shows for positive ion mode, a methyl shift [+14 Da] as shown in the base peak product ion (m/z 198.0888). FIG. 5B shows a double formate adduct anion for methylated SM 34:1; O2 were generated in negative ion mode, however acyl fatty acid product anions were not detected at this collision energy unlike methylated PCs.

FIG. 6A shows optimization of lipid methylation reaction conditions using FMOC/MTT as a function of reaction time that highlights a visible change in color intensity with longer reaction times. FIG. 6B shows a minimum reaction time of 60 min at 60° C. was determined to generate a quantitative and stable yield of methylated PCs based on analysis of 16 representative plasma PCs from NIST SRM-1950. FIG. 6C shows bar graphs that compare the average yield of methylated PCs (˜90%) in plasma extracts, where errors bars represent standard deviation (±1s, n=5). FIG. 6D shows representative extracted ion electropherograms highlighting the quantitative yield of methylated PCs without ion suppression, where reaction yields were assessed on native (underivatized) plasma PCs analyzed prior to and following FMOC/MTT labeling using NACE-MS with a single sample injection to improve their resolution from the EOF to avoid matrix-induced ion suppression effects in exemplary embodiments of the disclosure.

FIG. 7A shows electrophoretic mobility plot as a function of the accurate mass for 76 PLs measured in NIST SRM-1950 plasma extracts by MSI-NACE-MS under positive ion mode detection. A large mobility shift occurs following quantitative methylation, resulting in better separation resolution of both methylated PCs and SMs that are dependent on their chemical linkage, total fatty acyl chain carbon numbers, and degrees of unsaturation. FIG. 7B shows linear least-squares regression models were used to predict changes in the apparent electrophoretic mobility for plasma PLs as reflected by a homologous series of saturated and polyunsaturated PCs with the same total carbon chain length and as a function of increasing degrees of unsaturation. These distinctive mobility trends support the identification of unknown lipids in conjunction with MS/MS in exemplary embodiments of the disclosure.

FIGS. 8A,B show a comparison of MS/MS spectra acquired after collision-induced dissociation experiments in exemplary embodiments of the disclosure under positive (FIG. 8A) and negative ion (FIG. 8B) modes for methylated and native PC 40:6 from plasma extracts. This confirmed the methylation of the phosphatidylcholine head group as reflected by a characteristic methyl shift (+14 Da) when comparing the molecular ion and base peak/product ion under positive ion mode, whereas the fatty acyl chain backbone and their relative positioning under negative ion mode was consistent with PC 18:0_22:6.

FIG. 9 shows a representative MS/MS spectrum acquired for methylated PC 38:5 from NIST SRM-1950 plasma ether extract when using MSI-NACE-MS, which was tentatively identified as an EPA (20:5)-containing phospholipid in exemplary embodiments of the disclosure. However, collision-induced dissociation experiments at 20 V under negative ion mode subsequently confirmed that methylated PC 38:5 was in fact comprised of a mixture of two co-migrating phospholipids, namely PC (16:0_22:5) and PC (18:1_20:4) as major and minor species, respectively. As a result, not all plasma phospholipids annotated based on their sum composition represent fully resolved and unique lipid species.

FIGS. 10A,B show mobility maps depicting all methylated PC species (n=48) (FIG. 10A) and methylated SM species (n=27) (FIG. 10B) detected from NIST SRM-1950 human plasma extracts after FMOC/MTT derivatization using MSI-NACE-MS in positive ion mode with full-scan data acquisition in exemplary embodiments of the disclosure. Overall, there was a good linear correlation between the apparent electrophoretic mobility for single charged methylated phospholipids as a function of molecular weight (i.e., total carbon number) with deviations due to variations in degrees of unsaturation and specific chemical linkage that impact molecular volume within a phospholipid class, notably among highly unsaturated PCs.

FIG. 11 shows Venn diagram summarizing the coverage of consensus lipids from NIST SRM-1950 plasma ether extracts when using MSI-NACE-MS under positive (with FMOC/MTT derivatization) and negative ion mode (no derivatization) as reported by Bowden et al. (J. Lipid Res. 2017 58:2275) in exemplary embodiments of the disclosure. In this case, plasma PCs were annotated in the lipidomic harmonization study as their sum composition together with mass resolvable plasmanyl and plasmenyl PCs. However, the latter species were not detected in NIST SRM-1950 by MSI-NACE-MS. Due to the hexane back extraction clean up used to reduce ion suppression from excess MTT by-products, hydrophilic/polar PLs (PC<30, LPCs, PIs etc.) were better suited for their direct analysis by MSI-NACE-MS under negative ion mode without chemical derivatization. Other acidic lipid classes from plasma extracts that were not reported (e.g., PSs and PAs) or did not satisfy criteria in the lipidomics harmonization study have not been included, such as various FAs. However, electrically neutral lipid classes (e.g., cholesteryl esters, diacylglycerols) are not reliably quantified by MSI-NACE-MS under these conditions as they co-migrate with the EOF.

FIGS. 12A-C show detection of methylated PC species. FIG. 12A shows representative extracted ion electropherograms for methylated PC species when using distinct serial injection configurations in MSI-NACE-MS, including spike and recovery studies, serial dilution of NIST SRM-1950, and a serial dilution of calibrant solutions in exemplary embodiments of the disclosure. FIG. 12B shows the lack of ion suppression effects for methylated PC 40:6 and PC 38:6 was evident based on the good mutual agreement of their relative response factors or slope (i.e., mM⁻¹) acquired from 5-point calibration curves after serial dilution of PL standards or NIST SRM-1950 in exemplary embodiments of the disclosure. FIG. 12C shows inter-laboratory method comparison of PCs (n=20) and SMs (n=26) as consensus PLs from NIST SRM-1950 reported by Bowden et al. (J. Lipid Res. 2017 58:2275-2288). relative to their average concentrations measured by MSI-NACE-MS in exemplary embodiments of the disclosure. Plasma PL concentrations were estimated by performing a serial dilution of NIST SRM-1950 using their median of mean concentrations (>0.5 mM, COV<40%) to derive a response factor in MSI-NACE-MS among 21 quantifiable PLs (>4 calibrant points, Table 1). This strategy allowed for semi-quantification of plasma PLs by MSI-NACE-MS when standards were lacking. However, greater variability and bias was noted for lower abundance plasma PLs with an average bias of 103% (<5 mM, n=17), whereas an average bias of −9.7% was more acceptable for more abundant PLs (>5.0 mM, n=29). As expected, plasma SMs and PLs using surrogate lipids for response factor estimation were more prone to inaccuracy.

FIGS. 13A,B demonstrate a representative methodology of an exemplary embodiment of the disclosure. FIG. 13A shows overview of an accelerated data workflow for rapid identification of putative serum biomarkers of n3-LCPUFA intake following high-dose FO supplementation as compared to placebo/baseline when using MSI-NACE-MS with temporal pattern signal pattern recognition under two complementary configurations. FIG. 13B shows representative extracted ion electropherograms highlighting various PL classes/species that do not change following FO supplementation (e.g., SM 34:1; O2, LPC 20:5, PE 38:6) in contrast to specific serum PC species that undergo a notable increase after FO ingestion, such as PC 36:5. In all cases, a sub-group analysis was performed by MSI-NACE-MS under positive and negative ion modes when analyzing a serial injection of pooled serum samples from participants in the placebo/baseline and FO treatment arms in triplicate together with a blank extract as control. All lipids in serum extracts were annotated based on their accurate mass (m/z), relative migration time (RMT) and ionization mode (p or n) in exemplary embodiments of the disclosure.

FIGS. 14A-D show sub-group analysis of high-dose FO supplementation as compared to placebo/baseline showing response changes for EPA (FIG. 14A) and DHA (FIG. 14B) as their free NEFAs that were measured by MSI-NACE-MS under negative ion mode. FIG. 14C shows independent replication of increase to PC 36:5 (underivatized and detected as its acetate adduct) by MSI-NACE-MS following FO ingestion under negative ion mode, which was sub-optimal for quantitative analysis given impact of matrix induced ion suppression and overall lower sensitivity under these operating conditions. FIG. 14D shows a DHA-containing PC species (PC 38:6) was confirmed to have a major increase in response following FO ingestion albeit to a lesser extent than PC 36:5 when using MSI-NACE-MS under positive ion mode (with FMOC/MTT labeling; refer to FIG. 13B in exemplary embodiments of the disclosure.

FIGS. 15A,B show representative extracted ion electropherograms for methylated PC 36:5 (FIG. 15A) and PC 38:6 (FIG. 15B) and their corresponding full-scan TOF-MS spectra highlighting potential type-II isotopic effects in MSI-NACE-MS when using a seven serial sample injection under positive ion mode in exemplary embodiments of the disclosure. Both methylated PC 36:5 and PC 38:6 from plasma extracts did not have type-II isotopic effects due to the lack of co-migrating lipid isotopomers having one additional double bond (i.e., PC 36:6 or PC 38:7). In fact, the signal of the M+2 isomer for most other PC species are dominated by their co-migrating isotopomer having one additional double bond (>80%) except for PC 36:1, and PC 38:3 and PC 38:2.

FIGS. 16A,B show study of serum lipid profiles in women involved in a placebo-controlled trial involving long-chain omega-3 fatty acid supplementation. FIG. 16A shows the study design overview of serum samples from a cohort of young women ingesting either sunflower oil (SO) or high-dose fish oil (FO) supplement over a 56-day period. FIG. 16B shows a summary of lipidomic data structure and data quality when using a 2D PCA scores plot and 2D hierarchical cluster analysis heatmap based on the analysis of 44 serum PCs (as their methylated phosphoesters), including putative O3I biomarkers identified after the sub-group analysis. The technical precision based on repeat analysis of pooled QC serum samples was acceptable (median CV=13%, n=13) when compared to the biological variability of the serum lipidome (median CV=49%, n=69) as also demonstrated in the control chart for PC 36:5. Extracted ion electropherogram depicts changes in the ion response for a randomized series of serum extracts analyzed by MSI-NACE-MS under positive ion mode conditions. Samples representing FO supplementation are noted by their large ion responses as compared to baseline or placebo samples, whereas the QC represents a pooled average response for the entire cohort in exemplary embodiments of the disclosure.

FIGS. 17A-C show serum PC trajectory plots measured in young women (n=9) from baseline after ingesting high-dose fish oil (FO) supplement (3 g/day EPA+2 g/day DHA) as compared to sunflower oil (SO) as placebo over 56-day period in exemplary embodiments of the disclosure. FIG. 17A highlights two top-ranked serum PCs (PC 36:5; PC 38:5) that were most responsive to FO intake unlike a linoleic acid containing PC as control (PC 36:2). FIG. 17B highlights two top-ranked serum PCs (PC 40:6; PC 38:6) that were most responsive to FO intake unlike an oleic acid containing PC as control (PC 32:1). FIG. 17C shows a comparison of treatment response trajectories to high-dose FO intake based on the sum of three EPA (PC 36:5+PC 38:5+PC 40:5) or DHA (PC 36:6+PC38:6+PC 40:6) associated PCs as compared to the sum of only two EPA and DHA-specific lipid biomarkers (PC 36:5+PC 38:6). Error bars on plots represent ±1s as the biological variance that increased after FO supplementation relative to baseline or placebo. Table 7 summarizes statistical outcomes when using a two-way repeat measures ANOVA mixed model from this data, whereas lipid responses were reported in terms of their relative peak area (RPA) with signals normalized to PC 16:0[D62] as an internal standard.

FIGS. 18A, B show a comparison of two circulating lipid pools in young adult women and their correlation with O3I following high-dose FO supplementation versus placebo (SO) in exemplary embodiments of the disclosure. Scatter plot for serum ether extracts for the sum of PC 36:5+PC 38:6 with O3I (FIG. 18A) demonstrate a slightly stronger correlation to O3I with greater senstivity than the sum of EPA+DHA as their NEFAs (FIG. 18B). NEFA data was previously reported in serum ether extracts using MSI-NACE-MS under negative ion mode conditions as reported by Azab et al. (J. Lipid Res. 2020, 63:933-944).

FIGS. 19A,B show collision-induced dissociation MS/MS spectra acquired for methylated PC 36:5 (FIG. 19A) and PC 38:6 (FIG. 19B) identified as two circulating biomarkers of the O3I from serum extracts under positive and negative ion mode using a Q-TOF under an optimal collision energy of 20 V in exemplary embodiments of the disclosure. Annotation of MS/MS spectra confirm that a phosphatidyl choline head group likely containing palmitic acid (FA 16:0) and EPA (FA 20:5) or DHA (FA 22:6) in sn-1 and sn-2 positions, respectively based on their relative peak intensity (PC 16:0_20:5; PC 16:0_22:6). Chemical derivatization was performed using FMOC/MTT to render a positive charge on methylated PCs and improve their resolution and ion responses in MSI-NACE-MS under positive ion mode detection.

FIGS. 20A,B show calibration curves used for (semi)-quantification of lead omega-3 containing PCs responsive to high-dose fish oil and EPA or DHA supplementation in human serum or plasma extracts when using pre-column methylation prior to MSI-NACE-MS analysis under positive ion mode conditions in exemplary embodiments of the disclosure. Least-squares linear regression was performed with linearity over a 50 to 100-fold dynamic range when using an authentic lipid standard for PC 38:6 (or PC 16:0_22:6) (FIG. 20A), and a surrogate lipid standard (PC 36:4) for PC 36:5 (or PC 16:0_20:5) (FIG. 20B) based on serial dilution of NIST SRM-1950 (pooled human plasma) using consensus concentrations from lipidomics harmonization study by Bowden et al. (J. Lipid Res. 2017 58:2275-2288). All serum or plasma PCs were derivatized using FMOC/MTT to generate cationic phosphomethylesters to improve their separation resolution and ionization response under positive ion mode with full-scan data acquisition, with normalization to a single internal standard, PC 32:0[D62].

FIGS. 21A,B show a scatter plot with a strong linear correlation (r=0.738, n=69) between a serum PC (FIG. 21A) containing EPA (PC 36:5 or PC 16:0_20:5) with erythrocyte PL derived EPA measurements that is more sensitive to treatment responses following FO supplementation at three time intervals (28, 42 and 56 days), including detection of dietary non-adherence for a participant (S10) in exemplary embodiments of the disclosure. FIG. 21B shows there was a weaker linear correlation (r=0.381, n=69) for a serum PC containing DHA (PC 38:6 or PC 16:0_22:6) when compared to erythrocyte PL derived DHA measurements.

FIGS. 22A-C show representative spaghetti plots depicting individual treatment responses of top-ranking plasma PLs after treatment with either EPA (FIG. 22A), DHA (FIG. 22B) or olive oil (OO) (FIG. 22C) as placebo based on single lipid biomarkers and their sum in exemplary embodiments of the disclosure. Overall, PC 36:5 or PC 16:0_20:5 serves as a circulating biomarker most sensitive to EPA supplementation as compared to PC 38:6 or PC 16:0_22:6 following DHA ingestion at a similar dosage (3 g/day) after a 90-day period. No differences were measured in EPA or DHA-containing plasma PCs after ingestion of olive oil (OO). Median concentrations for plasma PCs at baseline and following treatment are reported along with between-subject variation (as a mean CV) in responses following treatment (or their difference from baseline in brackets).

FIGS. 23A,B show representative spaghetti plots for linoleic acid (FIG. 23A) and oleic acid (FIG. 23B) containing PCs (PC 36:1, PC 38:2) from plasma extracts that demonstrated only a modest increase from baseline in the olive oil (OO) placebo/control sub-group over 56 days in exemplary embodiments of the disclosure. As expected, this control sub-group did not exhibit significant increases in circulating omega-3 containing PCs (PC 36:5, PC 38:6).

FIGS. 24A,B show changes in the O3I status following high-dose DHA or EPA supplementation as compared to placebo. FIG. 24A shows a scatter plot showing strong correlation (r=0.764, p=3.0×10⁻³³) of the O3I to plasma concentrations of the sum of PC 36:5 and PC 38:6 at baseline/placebo and following EPA or DHA only supplementation (n=167). Established O3I cut-off intervals are stratified into three categories, where <4%, 4-8% and >8% are defined as high-, intermediate- and low-risk profiles for cardiovascular events. FIG. 24B shows overall, high-dose DHA supplementation contributed to a greater treatment response in terms of O3I status than EPA intake as depicted in the correlation plot between the sum concentration for these two plasma PCs to the change in O3I status at baseline for individual participants in exemplary embodiments of the disclosure.

FIG. 25 shows a correlation plot highlighting the strong linear relationship between circulating plasma PC 36:5+PC 38:6 expressed as a fraction of total PC (n=44) relative to erythrocyte PL membrane derived O3I measurements following high-dose EPA, DHA or olive oil (OO) supplementation as placebo from baseline in a cohort of young Canadian adults. Overall, the correlation strength to O3I was only modestly improved when using fraction (%) of PC 36:5+PC 38:6 as compared to their absolute concentrations (umol/L) in fasting human plasma. Plasma ether extracts were analyzed by MSI-NACE-MS under positive ion mode with full-scan data acquisition after lipid methylation using FMOC/MTT.

### DETAILED DESCRIPTION

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature described herein may be combined with any other feature or features described herein.

### I. Definitions

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies. In addition, all ranges given herein include the end of the ranges and also any intermediate range points, whether explicitly stated or not.

As used in this disclosure, the singular forms “a”, “an” and “the” include plural references unless the content clearly dictates otherwise. Thus, for example, a composition containing “a compound” includes a mixture of two or more compounds.

In embodiments comprising an “additional” or “second” component, the second component as used herein is chemically different from the other components or first component. A “third” component is different from the other, first, and second components, and further enumerated or “additional” components are similarly different.

The term “and/or” as used herein, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The abbreviation, “e.g.” is derived from the Latin exempli gratia and is used herein to indicate a non-limiting example. Thus, the abbreviation “e.g.” is synonymous with the term “for example.”

The recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

The present description refers to a number of chemical terms and abbreviations used by those skilled in the art. Nevertheless, definitions of selected terms are provided for clarity and consistency.

It will be understood that any component defined herein as being included may be explicitly excluded by way of proviso or negative limitation, such as any specific compounds or method steps, whether implicitly or explicitly defined herein.

All definitions, as defined and used herein, should be understood to control over dictionary definitions, definitions in documents incorporated by reference, and/or ordinary meanings of the defined terms.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary.

### II. Methods

Provided herein is a method of generating a lipid profile using mass spectrometry (MS), the method comprising:
a) extracting a lipid fraction from a sample obtained from a subject;
b) protecting the amine of one or more lipids in the lipid fraction using an amine protecting reagent to provide one or more protected lipids;
c) methylating a phosphate ester to generate a phosphate methyl ester of the one or more protected lipids using 3-methyl-1-p-tolyltriazene (MTT) to generate one or more methylated lipids;
d) optionally back extracting the one or more methylated lipids;
e) optionally separating the one or more methylated lipids;
f) introducing the one or more methylated lipids to a mass spectrometer under positive-ion mode; and
g) acquiring a mass spectrum chart of the one or more methylated lipids to generate the lipid profile of the sample.

As used herein, “mass spectrometry” or “MS” refers to an analytical technique used to identify the chemical composition and structure of a sample by measuring the mass-to-charge ratio (m/z) of its ionized particles. The method involves ionizing chemical compounds to generate charged molecules or molecular fragments, separating these ions based on their mass-to-charge ratios, and detecting them to produce a spectrum. This spectrum serves as a molecular fingerprint that can be analyzed to determine the identities and quantities of the components in a sample. Mass spectrometry techniques include a variety of ionization methods and mass analyzer types which may be combined. Examples of ionization methods include, without limitation, Electron Impact Ionization (EI), Chemical Ionization (CI), Electrospray Ionization (ESI), Matrix-Assisted Laser Desorption/Ionization (MALDI), Atmospheric Pressure Chemical Ionization (APCI), Atmospheric Pressure Photoionization (APPI), Fast Atom Bombardment (FAB), Desorption Electrospray Ionization (DESI), Secondary Ion Mass Spectrometry (SIMS), Field Ionization (FI), Field Desorption (FD) and multi-segment injection (MSI). Examples of mass analyzer types include, without limitation, Quadrupole Mass Analyzer, Time-of-Flight (TOF), Orbitrap, Magnetic Sector Analyzer, Ion Trap (including 3D and linear ion traps), Fourier Transform Ion Cyclotron Resonance (FT-ICR), Double-Focusing Mass Analyzer, Quadrupole Ion Trap (QIT), Hybrid Ion Trap-Orbitrap Systems, Dynamic Reaction Cell (DRC) for ICP-MS. Hybrid techniques include, for example, tandem mass spectrometry (MS/MS), including triple quadrupole and quadrupole time of flight (TOF), gas chromatography mass spectrometry (GS-MS), liquid chromatography mass spectrometry (LC-MS), inductively coupled plasma mass spectrometry (ICP-MS), capillary electrophoresis-mass spectrometry (CE-MS), MALDI-TOF, MALDI-TOF/TOF, and nonaqueous capillary electrophoresis-mass spectrometry (NACE-MS).

The term “positive ion mode” as used herein refers to a mass spectrometry technique in which the instrument detects and analyzes positively charged ions, such as amines, peptides, proteins and small organic compounds.

In some embodiments, in step e) separating comprises capillary electrophoresis, liquid chromatography or ion mobility

In some embodiments, extracting the lipid fraction in a) comprises incubating the sample with methyl tert butyl ether (MTBE) or related organic solvents, such as hexane, chloroform, methanol, acetonitrile.

As used herein, the term “lipid fraction” refers to the lipid portion of a sample, such as a blood sample (e.g., whole blood, dried blood spot, plasma, serum), as well as other specimens, such as cell or tissue extract. Methods of obtaining a lipid fraction are known to the skilled person and include, for example, the Bligh and Dyer method and the Folch method.

In some embodiments, step b) further comprises drying the one or more protected lipids under nitrogen.

The term “protecting” as used herein refers to a chemical modification to mask a functional group on a molecule, preventing it from reacting under certain conditions in a multi-step synthesis. Examples of amine protecting groups include, tert-butyloxycarbonyl (BOC), carbobenzoxy (Cbz) and FMOC. In some embodiments, the amine protecting reagent is 9-fluorenylmethyoxycarbonyl chloride (FMOC).

The terms “methylated” or “methylating” as used herein refers to a chemical modification in which a methyl (—CH₃) group is added to a molecule.

In some embodiments, the one or more methylated lipids is a cationic phosphate methyl ester lipid.

In some embodiments, the one or more lipids includes various lipid classes that can undergo methylation, such as phospholipids, glycerophospholipids, sphingolipids, glycerolipids, sterols, fatty acids, bile acids, steroids, isoprenoids, glycolipids, polyketides, saccharolipids, prenol lipids, sterols, a lipid containing a reactive amino, carboxyl, phenol, thiol, hydroxyl, or phosphate functionality (e.g., acylcarnitines, acyl-coenzyme A) or combinations thereof.

In some embodiments the one or more lipids is a phospholipid.

In some embodiments, the phospholipid is sphingomyelin (SM), phosphatidylcholine (PC), phosphatidylserine (PS), phosphatidylinositol (PI), phosphatidylethanolamine (PE), phosphatidic acid (PA), lysophosphatidylcholine (LPC), lysophosphatidylethanolamine (LPE), lysophosphatidic acid (LPA), cardiolipin (CL) or combinations thereof.

In some embodiments, the method further comprises in step e) introducing a known amount of one or more methylated lipids as a reference sample for calibration of lipid quantity, optionally the method further comprises generating an external calibration curve using a serial dilution of the reference sample.

In some embodiments, the PLs are quantified with improved resolution, sensitivity, and throughput compared to methylation using diazomethane.

In some embodiments, the one or more lipids in the lipid fraction in step b) are cationic or zwitterionic. In some embodiments, the one or more methylated lipids are cationic or zwitterionic, optionally the one or more methylated lipids are zwitterionic or cationic phospholipids.

In some embodiments, step c) has a reaction time of about 20 minutes to about 100 minutes, about 20 minutes to about 40 minutes, about 40 minutes to about 60 minutes, about 60 minutes to about 100 minutes, about 60 minutes to about 80 minutes, or about 40 minutes to about 80 minutes, optionally the reaction time is about 60 minutes.

In some embodiments, the concentration of MTT in step c) is about 50 mM to about 900 mM, optionally, the MTT is at a concentration of about 450 mM.

In some embodiments, MTT represents a safer and more convenient alternative to diazomethane.

In some embodiments, step c) has a reaction temperature of about 20° C. to about 100° C., optionally the reaction temperature is about 60° C.

In some embodiments, step b) has a reaction time of about 1 minute to about 30 minutes.

In some embodiments, step b) has a reaction time of about 5 minutes.

In some embodiments, the concentration of the amine protecting reagent, optionally FMOC, in step b) is about 0.10 mM to about 10 mM.

In some embodiments, the concentration of the amine protecting reagent, optionally FMOC, in step b) is about 0.85 mM.

In some embodiments, the mass spectrometer comprises multisegment injection-nonaqueous capillary electrophoresis-mass spectrometry (MSI-NACE-MS), direct infusion-MS, desorption ionization (DESI)-MS, gas chromatography (GC)-MS, ion mobility (IM)-MS and liquid chromatography (LC)-MS and supercritical fluid chromatography (SFC)-MS.

In some embodiments, the mass spectrometer is multisegment injection-nonaqueous capillary electrophoresis-mass spectrometry (MSI-NACE-MS).

In some embodiments, the sample is of animal, plant or human origin. In some embodiments, the sample is from human blood, optionally plasma or serum.

In some embodiments, in step b) the amine protecting reagent, optionally FMOC, is added in excess and the excess amine protecting reagent reacts with p-toluidine and/or phosphatidylethanolamine (PE).

In some embodiments, the amine protecting reagent, optionally FMOC, reduces isobaric interferences and ion suppression effects. In some embodiments, the back extracting in d) reduces isobaric interferences and ion suppression effects.

In some embodiments, back extracting the one or more methylated lipids in d) comprises back extracting with hexane. In some embodiments, back extracting the one or more methylated lipids in d) comprises back extracting with methyl tert butyl ether (MTBE).

In some embodiments, the lipid profiling is targeted. In some embodiments, the lipid profiling is untargeted.

The term “untargeted”, as used herein refers to a comprehensive profile of the entire lipidome of a sample without prior knowledge of the lipids present.

The term “targeted” as used herein refers to the identification and quantification of a specific set of known lipids of interest. This technique may be used for absolute or relative quantification using standards or internal references.

In some embodiments, the method further comprises separating a portion of the lipid fraction obtained in step a) for mass spectrometry in negative ion mode. In some embodiments, negative ion mode is for detecting anionic lipids and a mass spectrum chart is generated and results are combined with the lipid profile of the sample in g).

The term “negative ion mode” as used herein refers to a mass spectrometry technique in which the instrument detects and analyzes negatively charged ions, such as carboxylic acids, phenols and phosphates.

Another aspect of the disclosure is a method of assessing omega-3 index (O3I) status in a subject, the method comprising:
a) obtaining a first sample from the subject at a first time point;
b) measuring the level of one or more omega-3 containing phospholipid biomarkers in the first sample; and
c) comparing the level of the one or more omega-3 containing phospholipid biomarkers in the first sample to a control level or value of the one or more omega-3 containing phospholipid biomarkers of known O3I status;
   wherein similarity of level of the phospholipid biomarkers in the sample with the control indicates that the sample is from a subject with the known O3I status.

As used herein, the omega-3 index (O3I), is defined as the erythrocyte (red blood cells (RBCs)) EPA+DHA content from the phospholipid (PL) fraction as a mole percent to total fatty acids, and represents a novel biomarker of coronary heart disease risk and sudden cardiac death independent of traditional risk factors (75). The O3I status can be stratified based on clinically defined cut-off values, where <4% is considered high risk, 4-8% being intermediate risk, and low risk >8% for mortality from coronary heart disease (76), as confirmed in a meta-analysis from 10 cohort studies (77). For example, the mean O3I index for Canadian adults has been reported as 4.5% with less than 3% classified as having high cardioprotection (i.e., O3I>8%) that was dependent on age, ethnicity, fish consumption, supplement use, smoking status and obesity (78). The O3I is calculated using the formula 1:

$$O 3 I \% = \frac{[ EPA + DHA in RBCs ]}{Total Fatty Acids in RBCs} \times 100 \qquad Formula 1$$

The term “docosahexaenoic acid” or “DHA”, as used herein, refers to an omega-3 fatty acid 22:6 (n−3). DHA is commonly found in cold water fish, such as salmon, or can be taken as a dietary supplement. DHA has the following chemical structure:

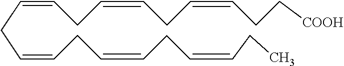 [SMILES: CC/C=C\C/C=C\C/C=C\C/C=C\C/C=C\C/C=C\CCC(=O)O]

The term “eicosapentaenoic acid” or “EPA”, as used herein, refers to an omega-3 fatty acid 20:5 (n−3). EPA is commonly found in oily fish, such as herring, mackerel, salmon or in edible algaes, or can be taken as a dietary supplement. EPA has the following chemical structure:

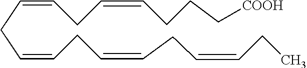 [SMILES: CC/C=C\C/C=C\C/C=C\C/C=C\C/C=C\CCCC(=O)O]

The term “control”, as used herein refers to a comparative sample, such as a blood sample, taken from a subject with a known O3I status, or a specific value or dataset that can be used to prognose or classify the value e.g., phospholipid biomarker level or reference phospholipid biomarker value obtained from a test sample or samples associated with a known O3I status. In one embodiment, the dataset may be obtained from samples of a group of subjects known to have an O3I of less than 4%, an O3I of 4%-8%, and/or an O3I of greater than 8%. The level of the phospholipid biomarkers in the dataset can be used to create a “control value” that is used in testing samples from new subjects. A control value may be obtained from historical phospholipid biomarker levels for a subject or pool of subjects with a known O3I status.

The term “subject” as used herein includes all members of the animal kingdom including mammals, and suitably refers to humans. Optionally, the term “subject” includes healthy mammals. In some embodiments, the term “subject” includes mammals that are taking dietary fish oil, docosahexaenoic acid (DHA) or eicosapentaenoic acid (EPA) supplementation or other dietary long-chain omega-3 fatty acids, including docosapentaenoic acid (DPA) and a-linolenic acid (ALA), as well as various natural lipids or synthetic analogs, such as ethyl eicosapentaenoic acid. In one embodiment, the term “subject” refers to a human having, or suspected of having, cardiovascular disease.

In some embodiments, the subject is a healthy subject. In some embodiments, the subject has, is suspected of having or is at risk of developing a cardiovascular disease. In some embodiments, the subject has, is suspected of having or is at risk of developing a cardiometabolic disorder. In some embodiments, the subject has, is suspected of having or is at risk of developing a neurodegenerative disorder. In some embodiments, the subject has, is suspected of having or is at risk of developing a mental health disorder. In some embodiments, the subject has, is suspected of having or is at risk of developing an autoimmune disorder. In some embodiments, the subject is, is suspected of being, or will become pregnant.

In some embodiments, the method is for monitoring O3I status in a pregnant subject. In some embodiments, the method is for monitoring prenatal supplementation and nutrition in a pregnant subject or in a subject who is planning to become pregnant.

The term “cardiometabolic disorder” as used herein refers to a condition or group of conditions characterized by one or more abnormalities in cardiovascular and/or metabolic systems, including but not limited to hypertension, dyslipidemia, obesity, insulin resistance, impaired glucose tolerance, diabetes, and associated systemic inflammation.

The term “neurodegenerative disorder”, includes any and all disorders and conditions of the central nervous system that involve neural degeneration and/or neural cell loss, including but not limited to Alzheimer's disease (AD), Parkinson's disease (PD), Amyotrophic Lateral Sclerosis (ALS), Huntington's disease (HD), Multiple Sclerosis (MS), cognitive decline and mild cognitive impairment (MCI).

The term “mental health disorder” refers to a condition characterized by disturbances in a person's cognition, emotional regulation or behavior, reflecting a dysfunction in psychological, biological, or developmental processes underlying mental function. Mental disorder includes, without limitation, mood disorders, depression, anxiety disorders, psychotic disorders, post-traumatic stress disorder, eating disorders, neurodevelopmental disorders and personality disorders.

The term “autoimmune disorder” refers to conditions where the immune system mistakenly attacks the body's own tissue leading to chronic or acute inflammation. Examples of autoimmune disorders associated with inflammation include, rheumatoid arthritis, systemic lupus erythematosus, Sjogren's Syndrome, Mixed Connective Tissue Disease, Hashimoto's Thyroiditis, Type 1 Diabetes, Inflammatory Bowel Disease (e.g. Crohn's disease and ulcerative colitis), multiple sclerosis, neuromyelitis optica, vasculitis, psoriasis, vitiligo, ankylosing spondylitis, dermatomyositis, polymyositis, and autoimmune hepatitis.

In some embodiments, the one or more omega-3 containing phospholipid biomarkers that correlate to O3I status comprise one or more phosphatidylcholines (PCs). In some embodiments, the one or more PCs are selected from the group consisting of PC 38:6(16:0_22:6), PC 36:5 (16:0_20:5), PC 38:5, PC 40:6, PC 36:6, and PC 40:5.

In some embodiments, the one or more PCs comprise one PC, two PCs, three PCs, four PCs, five PCs or six PCs.

In some embodiments, the one or more PCs comprise PC 36:5 (16:0_20:5) and PC 38:6 (16:0_22:6). In some embodiments the one or more PCs comprise PC 36:5 (16:0_20:5) and PC 38:6 (16:0_22:6) with chemical derivatization. In some embodiments, the one or more PCs comprise PC 36:5 (16:0_20:5) and PC 38:6 (16:0_22:6) without chemical derivatization.

In some embodiments, the one or more omega-3 containing phospholipid biomarkers that correlate to O3I status consist of two PCs, wherein the two PCs consist of PC 36:5 (16:0_20:5) and PC 38:6 (16:0_22:6).

In some embodiments, the one or more omega-3 containing phospholipid biomarkers that correlate to O3I status are PCs comprising omega-3 fatty acids containing eicosapentaenoic acid (EPA, 20:5) docosahexaenoic acid (DHA, 22:6), docosapentaenoic acid (DPA) and/or alpha-linolenic acid (ALA) together with other fatty acyl chains (e.g., 18:0).

In some embodiments, the omega-3 fatty acids comprise DHA and/or EPA.

In some embodiments, the levels of omega-3 containing PCs in circulating phospholipids are correlated with omega-3 index when adjusted for relative abundances between omega-3 containing circulating PCs compared to red blood cell membrane omega-3 index measurements

In some embodiments, the method further comprises repeating the method of assessing omega-3 index (O3I) status in a subject for a second sample from the same subject taken at a second time point to assess O3I status and monitor change from the first time point to the second time point.

In some embodiments, the first sample and/or the second sample comprises serum, plasma, whole blood or dried blood.

In some embodiments, assessing the level of one or more omega-3 containing phospholipid biomarkers may be measured by any suitable method known to the skilled person, such as immunoassays, including for example, enzyme linked immunosorbent assay (ELISA).

In some embodiments, assessing the level of one or more omega-3 containing phospholipid biomarkers comprises a method of chemical derivatization using the methods described herein.

Another aspect of the disclosure is a method of assessing cardiovascular risk in a subject, the method comprising: assessing omega-3 index (O3I) status in a subject using the methods described herein, wherein if the level of the one or more omega-3 containing phospholipid biomarkers is similar to a control of less than 4% O3I the subject is determined to be at high cardiovascular risk, if the level of the one or more omega-3 containing phospholipid biomarkers is similar to a control of 4% to 8% O3I the subject is determined to be at intermediate cardiovascular risk, and if the level of the one or more omega-3 containing phospholipid biomarkers is similar to a control of more than 8% 031 the subject is determined to be at low cardiovascular risk.

Methods of determining the similarity between profiles are well known in the art. Methods of determining similarity may in some embodiments provide a non-quantitative measure of similarity, for example, using visual clustering. In other embodiments, similarity may be determined using methods which provide a quantitative measure of similarity.

In some embodiments, if the subject has a high or intermediate cardiovascular risk, the method further comprises treating the subject by administering omega-3 fatty acid supplementation.

In some embodiments, the method further comprises re-assessing the cardiovascular risk in a subsequent sample following omega-3 fatty acid supplementation.

In some embodiments, the omega-3 fatty acid supplementation comprises fish oil, eicosapentaenoic acid (EPA) and/or docosahexaenoic acid (DHA).

In some embodiments, the subsequent sample is obtained about 1 day to about 90 days after initiation of supplementation. The timing of obtaining the subsequent sample can be determined by the skilled person and may be determined based on the formulation of the supplementation, the frequency of dosing and the dosage.

In some embodiments, the subsequent sample is obtained about 28 days after initiation of supplementation.

In some embodiments, the method further comprises adjusting the omega-3 fatty acid supplementation based on the change in level of the one or more phospholipid biomarkers that correlate to O3I status from the first time point to the second time point.

In some embodiments, adjusting the omega-3 fatty acid supplementation comprises increasing the omega-3 fatty acid supplementation if the subject is determined to be at high cardiovascular risk or at intermediate cardiovascular risk.

In some embodiments, adjusting the omega-3 fatty acid supplementation comprises maintaining or discontinuing the omega-3 fatty acid supplementation if the subject is determined to be at low cardiovascular risk.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease (e.g. maintaining a subject in remission), preventing disease or preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein can also mean mitigating the risk of or the risk of developing cardiovascular in a subject.

Another aspect of the disclosure is a method of treating and/or preventing a mental health disorder, an autoimmune disorder, or a neurodegenerative disorder in a subject, the method comprising:
a) assessing omega-3 index (O3I) status in a subject using the methods described herein; and
b) (i) administering omega-3 fatty acid supplementation if the level of the one or more phospholipid biomarkers is similar to a control of less than 4% 031;
(ii) administering omega-3 fatty acid supplementation if the level of the one or more phospholipid biomarkers is similar to a control of 4% to 8% O3I; or
(iii) advising the subject not to have omega-3 supplementation if the level of the one or more phospholipid biomarkers is similar to a control of more than 8% 031.

In some embodiments the mental health disorder is depression.

In some embodiments, the omega-3 fatty acid supplementation comprises fish oil, eicosapentaenoic acid (EPA) and/or docosahexaenoic acid (DHA).

In some embodiments, the method further comprises repeating the method of assessing O3I status for a second sample from the same subject taken at a second time point following omega-3 fatty acid supplementation.

In some embodiments, the subsequent sample is obtained about 1 day to about 90 days after initiation of supplementation. The timing of obtaining the subsequent sample can be determined by the skilled person, and may be determined based on the formulation of the supplementation, the frequency of dosing and the dosage

In some embodiments, the second sample is obtained about 28 days after initiation of supplementation.

In some embodiments, the method further comprises adjusting the omega-3 fatty acid supplementation based on the change in level of the one or more phospholipid biomarkers that correlate to O3I status from the first time point to the second time point.

In some embodiments, the impact of changes in omega-3 fatty acid intake through diet or supplements can be monitored over time, as well as risk assessment for incidence of clinical events (e.g., heart failure, stroke, cognitive decline).

The preceding section is provided by way of example only and is not intended to be limiting on the scope of the present disclosure and appended claims. Additional objects and advantages associated with the compositions and methods of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional advantages, objects and embodiments are expressly included within the scope of the present disclosure. The publications and other materials used herein to illuminate the background of the disclosure, and in particular cases, to provide additional details respecting the practice, are incorporated by reference, and for convenience are listed in the appended reference section.

### EXAMPLES

The following non-limiting examples are illustrative of the present disclosure:

### Example 1

Classical methods for lipid profiling of biological samples have relied on the analysis of esterified fatty acids from lipid hydrolysates using gas chromatography (GC)-MS.¹⁹ However, comprehensive analysis of intact phospholipids (PLs) was first achieved by MS when using soft ionization methods based on matrix-assisted laser desorption/ionization and electrospray ionization (ESI).²⁰ Although shotgun lipidomics enables the direct analysis of lipid extracts by direct infusion (DI)-MS,²¹ high efficiency separations are often needed to improve method selectivity while reducing ion suppression effects, isobaric interferences and/or various other mass ambiguities.²² To date, liquid chromatography (LC)-MS remains the separation platform of choice in lipidomics.²³ However, LC-MS protocols vary substantially in terms of operation conditions (e.g., column types, elution conditions etc.) used to resolve different lipid classes primarily by reversed-phase, normal-phase and/or hydrophilic interaction chromatography (HILIC).^{24,25} For instance, greater sample throughput, separation resolution and/or reproducibility can be achieved in reversed-phase LC-MS lipidomic analyses using core shell particles,²⁶ vacuum jacked columns,²⁷ capillaries operated under ultra-high pressure conditions,²⁸ and via multidimensional separations.²⁹ Alternatively, supercritical fluid chromatography-MS can resolve lipids that vary widely in their polarity with better robustness than HILIC-MS.³⁰ Also, ion mobility-MS enables the ultra-fast separation of PLs as compared to chromatographic methods with adequate selectivity to generate a lipidome atlas.³¹ On the other hand, nonaqueous capillary electrophoresis-mass spectrometry (NACE-MS) is largely an unrecognized separation technique in lipidomics likely due to a paucity of published studies limited to certain ionic lipids, such as saturated fatty acids³² lipid A isomers³³ and glycerophospholipids.^{34,35} Indeed, a lack of robust NACE-MS protocols, limited vendor support, and sparse method validation relative to existing chromatographic methods have deterred its use as a viable separation platform in untargeted lipid profiling.

Recently, multisegment injection (MSI)-NACE-MS was introduced as a multiplexed separation platform for the quantitative determination of fatty acids from blood specimens,^{6,36,37} which can also resolve other classes of anionic lipids under negative ion mode detection, such as phosphatidic acids and phosphatidylinositols.³⁸ Serial injection of seven or more samples within a single capillary allows for higher sample throughput³⁹ together with temporal signal pattern recognition in ESI-MS⁴⁰ for rigorous molecular feature selection and lipid authentication when performing nontargeted screening.³⁸ However, separation resolution and selectivity is currently limited for phosphatidylcholines (PC) and other classes of zwitter-ionic lipids that migrate close to the electroosmotic flow (EOF). Pre-column chemical derivatization strategies have been developed to introduce or switch charge states on specific lipid classes to modify their chromatographic retention, reduce isobaric interferences, and improve ionization efficiency with lower detection limits in ESI-MS.⁴¹ For instance, Smith et al.⁴²⁻⁴⁴ have used diazomethane for charge inversion on modified cationic PLs via quantitative methylation. However, given the explosive and toxicity hazards of diazomethane that is generated in-situ,⁴⁵ safer methylating agents are required in routine MS-based lipidomic workflows without blast shields and other personal protective equipment.

### Methods

Ultra LC-MS grade methanol, acetonitrile, water and 2-propanol were used to prepare the sheath liquid and the background electrolyte (BGE). Ammonium formate, formic acid, 1,2-distearoyl-d70-sn-glycero-3-phosphocholine (PC 36:0[D70]), 1,2-dipalmitoyl-d62-sn-glycero-3-phosphocholine (PC 32:0[D62]), methyl-tert-butyl ether (MTBE), MTT, FMOC and all other chemical standards were purchased from Sigma-Aldrich Inc. (St. Louis, MO, USA) unless otherwise stated. All lipid standards purchased were either as a powder or dissolved in solution (1:1) of chloroform and methanol. Stock solutions for lipids were then diluted in chloroform and methanol and stored at −80° C. prior to further use. Reference material from the National Institute of Standards and Technology (NIST) SRM-1950 pooled human plasma was purchased from the NIST (Gaithersburg, ML, USA). While certified reference values for NIST SRM-1950 have been reported for several polar metabolites, plasma PLs measured in this study were compared to the median of mean concentrations reported for NIST SRM-1950 in an international study across 31 laboratories that adopted various LC-MS/MS lipidomic workflows. In this case, consensus plasma PL concentrations required measurements from a minimum of 5 laboratories having a sample coefficient of dispersion (COD)<40%.¹⁵

### Plasma Lipid Extraction Using MTBE:

Plasma samples and lipid calibrant solutions were extracted using a modified MTBE-based liquid extraction procedure previously described for fatty acids and anionic lipids using MSI-NACE-MS in negative ion mode.^{36,38} Briefly, 50 μL of a NIST SRM-1950 plasma aliquot was mixed with 100 μL of methanol containing PC 32:0[D62] as a recovery standard and shaken for 10 min. Then, 250 μL of MTBE was added and the mixture was subject to vigorous shaking for 10 min. To induce phase separation, 100 μL of deionized water was then added prior to centrifugation at 10 min at 4000 g. Next, 200 μL of the lipid-rich MTBE upper layer was transferred into another vial and dried down at room temperature using an Organomation MULTIVAP® nitrogen evaporator (Berlin, MA, USA). For underivatized lipids, dried plasma extracts were then reconstituted to a volume of 50 μL containing acetonitrile/isopropanol/water (70:20:10) with 10 mM ammonium formate containing internal standards PC 36:0[D70] (5 μM), benzyltriethylammoniumchloride (BTA) (1 μM), and of PC 32:0[D62] (5 μM) prior to analysis by MSI-NACE-MS.

### Chemical Derivatization of Zwitterionic Phospholipids Using FMOC and MTT:

All plasma ether extracts and PL calibrants were subject to a two-step chemical labeling procedure using FMOC and MTT. In 2 mL amber glass vials, 100 μL of 0.85 mM FMOC in chloroform was added to dried ether plasma extracts and shaken vigorously for 5 min. Then, samples were blown down to dryness using nitrogen at room temperature prior to reconstitution in 50 μL of MTBE containing 450 mM of MTT. Vials were next sealed with Teflon tape and vortexed for 30 s prior to derivatization at 60° C. for 60 min (unless otherwise stated). Afterwards, 100 μL of MeOH, 250 μL of hexane and 200 μL of deionized water was added to back extract polar by-products of the reaction (e.g., p-toluidine). After centrifuging for 10 min at 4000 g, 200 μL of hexane as the supernatant was transferred out to a separate glass vial and then evaporated to dryness under nitrogen. Lastly, derivatized extracts were then reconstituted in 50 μL of acetonitrile/isopropanol/water (70:20:10) with 10 mM ammonium formate containing internal standards PC 36:0[D70] (5 μM), BTA (1 μM), and of PC 32:0[D62] (5 μM) prior to analysis by MSI-NACE-MS. Derivatization yields for methylated PLs from plasma extracts were calculated based on the integrated relative peak area (RPA) for each native (unlabelled) PL relative to PC 36:0[D70] as an internal standard using equation (1):

$$\% Derivatization Yield = 100 * ( 1 - \frac{FMOC \& MTT treated PL RPA}{Untreated PL RPA} ) \qquad ( 1 )$$

### CE-MS Instrumentation and Serial Injection Configuration:

An Agilent 6230 time-of-flight (TOF) mass spectrometer with a coaxial sheath liquid electrospray (ESI) ionization source equipped with an Agilent G7100A CE unit was used for all experiments (Agilent Technologies Inc., Mississauga, ON, Canada). An Agilent 1260 Infinity isocratic pump and a 1260 Infinity degasser were utilized to deliver an 80:20 MeOH-water with 0.1% vol formic acid at a flow rate of 10 μL/min using a CE-MS coaxial sheath liquid interface kit. For mass correction in real-time, the reference ions purine and hexakis (2,2,3,3-tetrafluoropropoxy) phosphazine (HP-921) were spiked into the sheath liquid at 0.02% vol to provide constant mass signals at m/z 121.0509 and 922.0098, which were utilized for monitoring ion suppression and/or enhancement effects. During sample introduction into the capillary, the nebulizer gas was turned off to prevent siphoning effects that may contribute to air bubbles and current errors upon voltage application.³⁶ This was subsequently turned on at a low pressure of 4 psi (27.6 kPa) following voltage application with the ion source operating at 300° C. with a drying gas of nitrogen that was delivered at 4 L/min. The TOF-MS was operated in 2 GHz extended dynamic range under positive mode detection. A V_{cap} was set at 3500 V while the fragmentor was 120 V, the skimmer was 65 V and the octopole rf was 750 V. All separations were performed using bare fused-silica capillaries with 50 μm internal diameter, a 360 μm outer diameter, and 100 cm total length (Polymicro Technologies Inc., AZ). A capillary window maker (MicroSolv, Leland, NC) was used to remove 7 mm of the polyimide coating on both ends of the capillary to prevent polyimide swelling with organic solvents in the background electrolyte (BGE) or aminolysis under alkaline nonaqueous buffer conditions.⁴⁶ An applied voltage of 30 kV was used for CE separations at 25° C. together while using a forward pressure of 5 mbar (0.5 kPa). The BGE was 35 mM ammonium formate in 70% vol acetonitrile, 15% vol methanol, 10% vol water and 5% vol isopropanol with an apparent pH of 2.3 adjusted with the addition of formic acid. Derivatized plasma extracts and lipid standards were introduced in-capillary hydrodynamically at 50 mbar (5 kPa) alternating between 5 s for each sample plug and 40 s for the BGE spacer plug for a total of seven discrete samples analyzed within a single run.³⁸ Prior to first use, capillaries were conditioned by flushing at 950 mbar (95 kPa) with methanol, 0.1 M sodium hydroxide, deionized water, and BGE sequentially for 15 min each. The BGE and sheath liquid were degassed prior to use. For analysis of NIST SRM-1950 by MSI-NACE-MS in negative ion mode to verify acidic lipids not amenable by the FMOC/MTT labelling, an alkaline BGE with the same organic solvent composition was used, but with ammonium acetate and ammonium hydroxide as the BGE and pH modifier respectively as described elswhere.³⁶ In this case, the same MTBE extraction protocol was applied for the direct analysis of fatty acids and anionic lipids, but the extract was concentrated two-fold without FMOC/MTT chemical derivatization. Plasma PLs were annotated by MSI-NACE-MS based on their sum composition, mass error and relative migration times (RMTs) or apparent electrophoretic mobilities (Table 1, 2) with select PLs from NIST SRM-1950 ether extracts further characterized by MS/MS for confirmation of molecular PC and SM species.

### Results and Discussion

### Separation Performance Enhancement After Phospholipid Methylation:

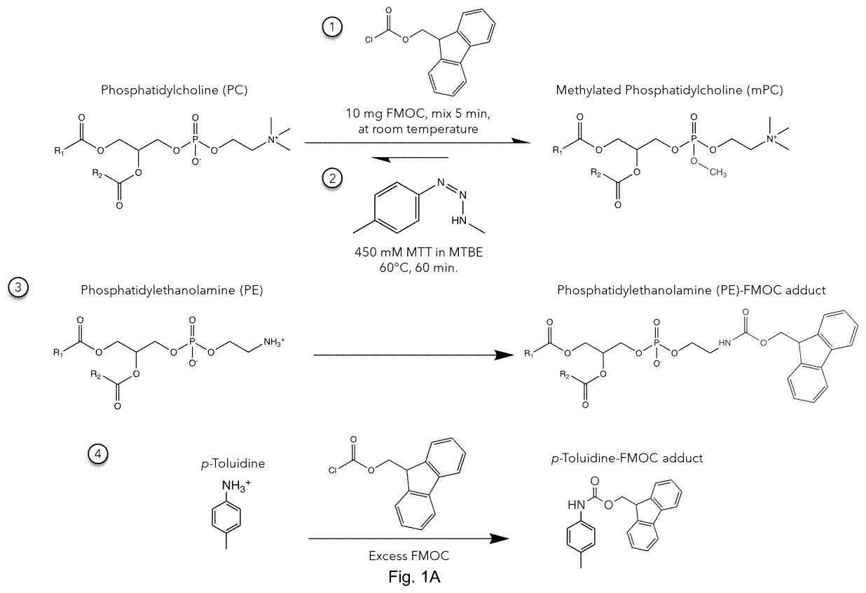

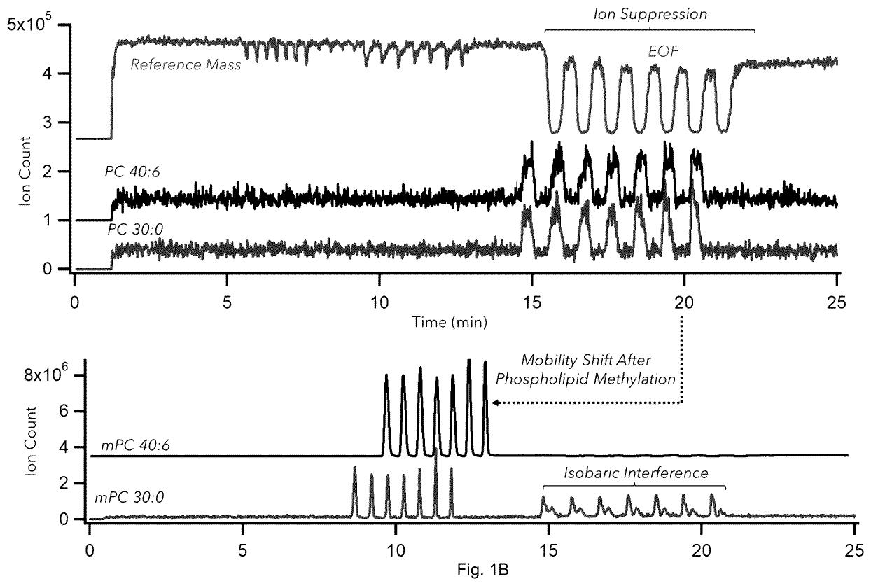

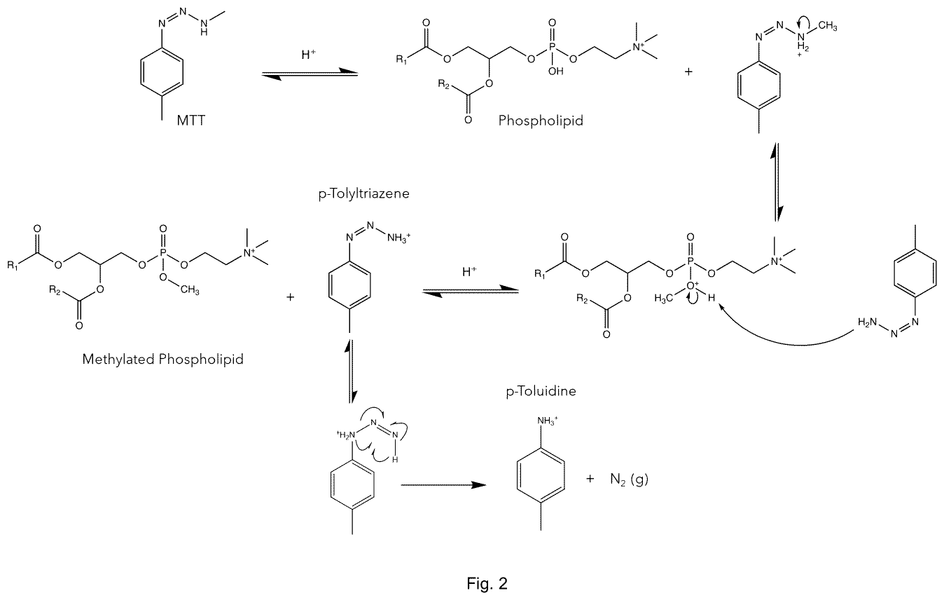

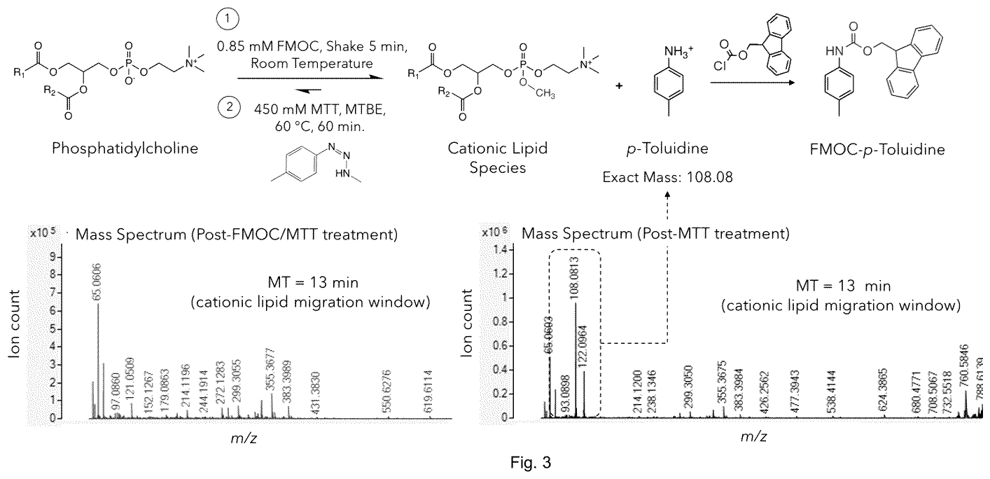

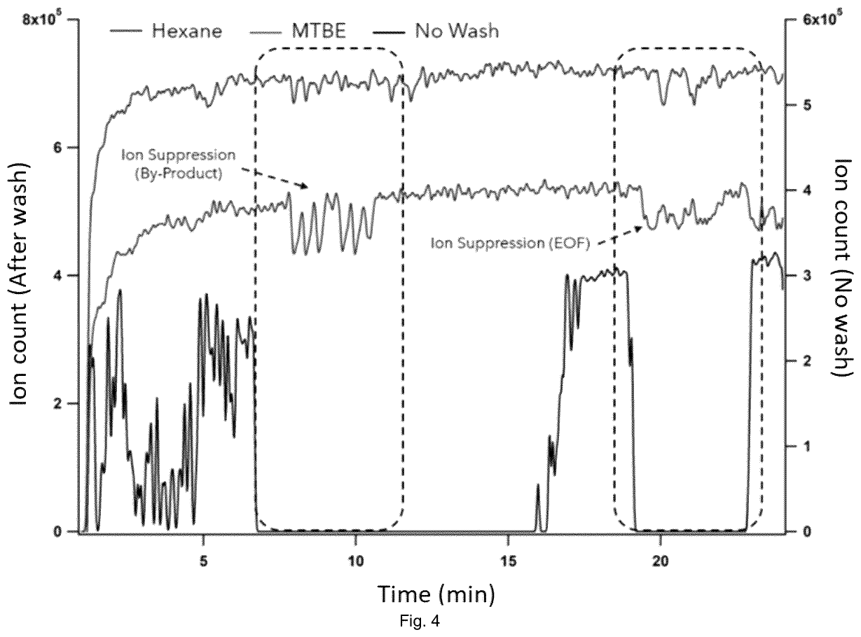

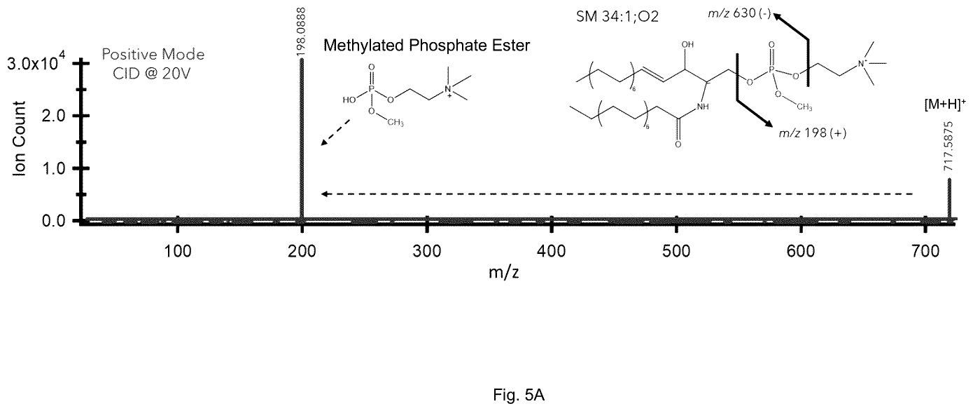

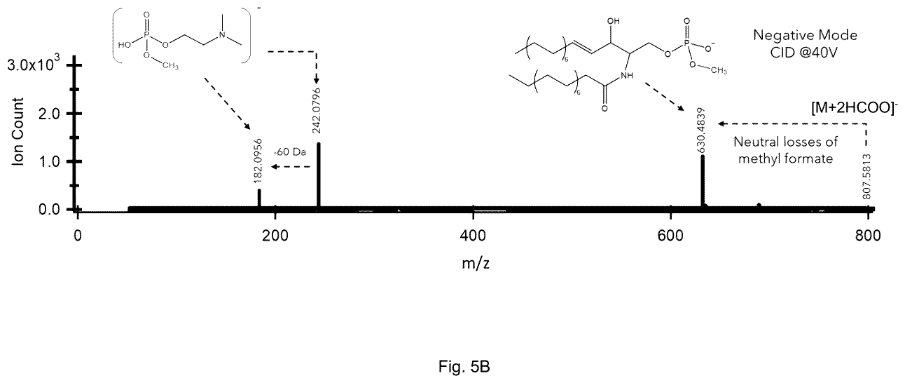

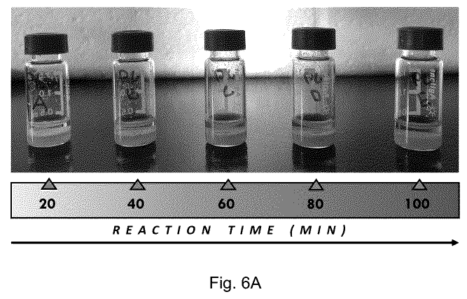

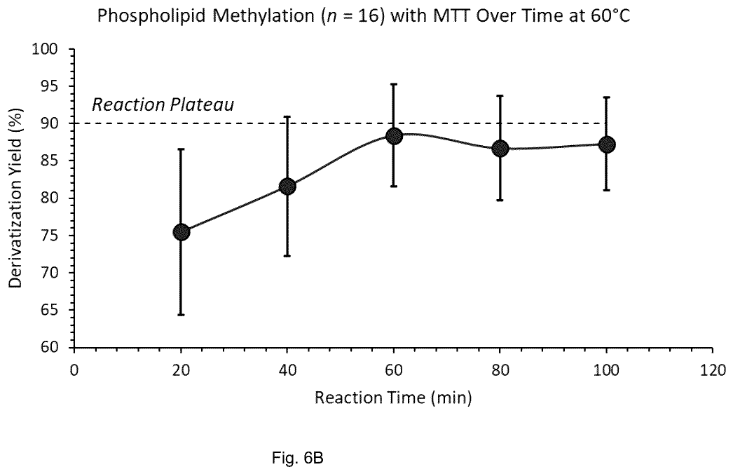

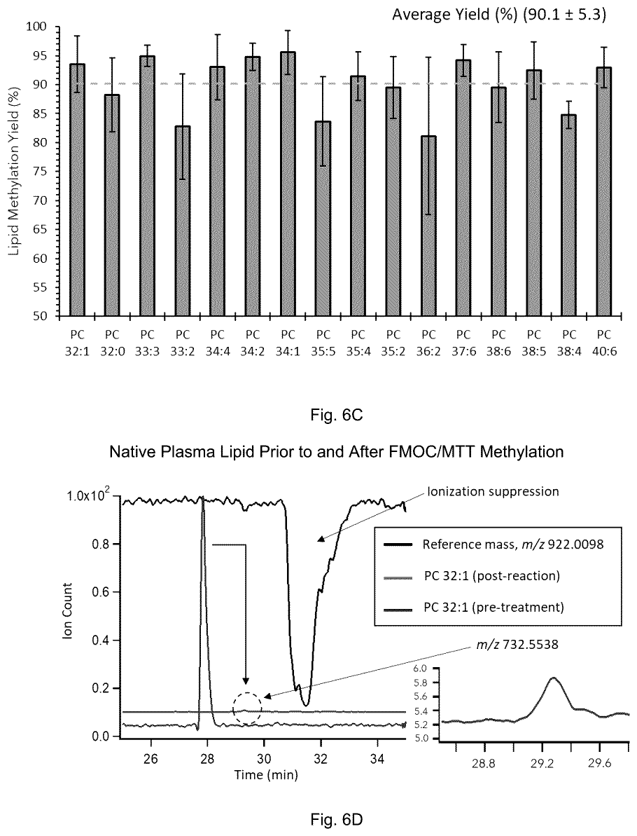

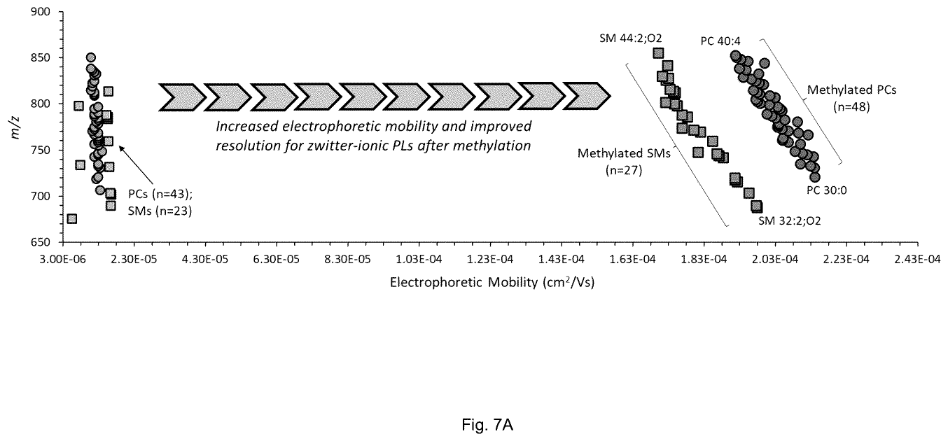

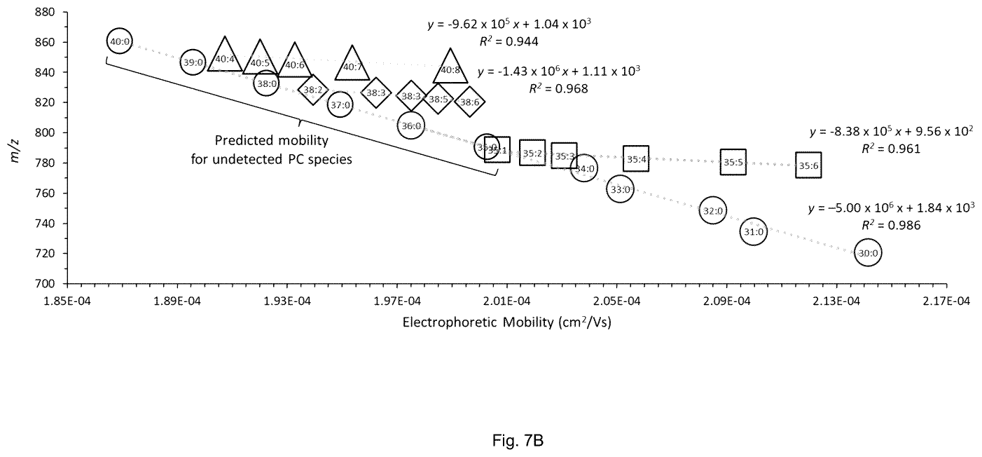

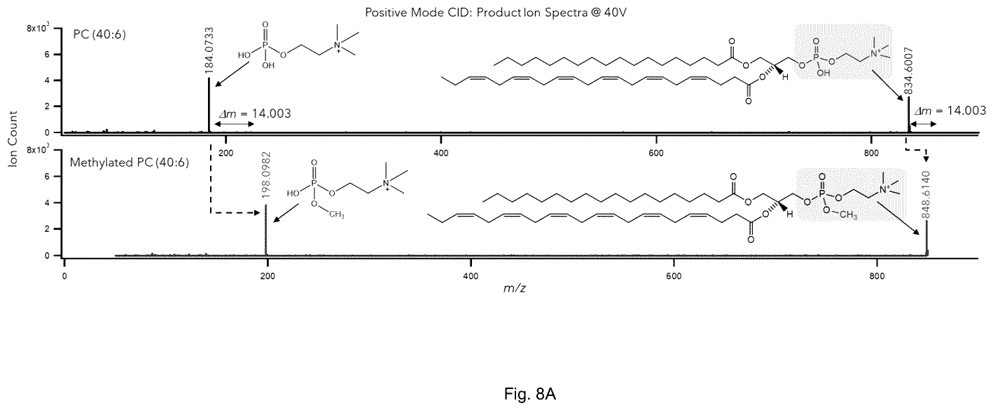

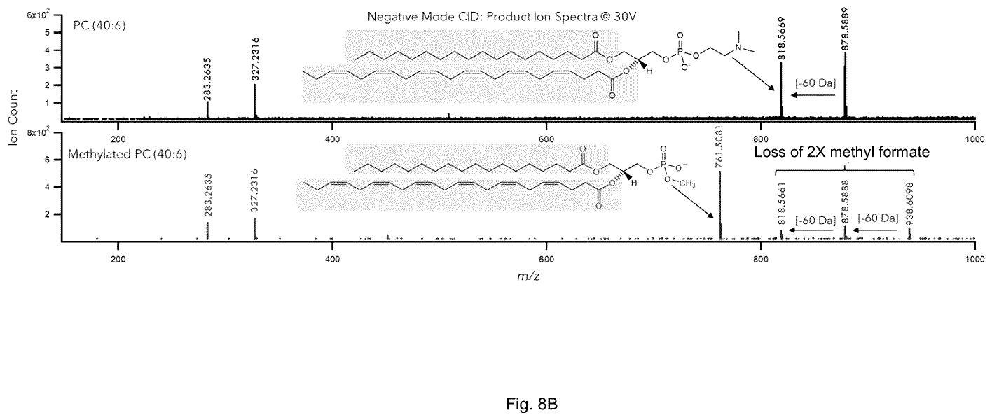

A two-step chemical labeling strategy using FMOC/MTT was first developed to generate a positive charge on methylated PLs to increase their electrophoretic mobility as depicted in FIG. 1A. FMOC was first added as a protecting agent to rapidly react (<5 min) with phosphatidylethanolamines (PEs) from plasma ether extracts since they can generate isobaric interferences with analogous PCs following their permethylation.⁴⁴ In this case, MSI-NACE-MS under alkaline buffer conditions and negative ion mode can directly analyze native PEs and other acidic lipids without chemical derivatization.³⁸ FMOC not only reacts with PE species from plasma ether extracts, but also with excess MTT by-product (i.e., p-toluidine) to form a neutral adduct as shown in the proposed reaction mechanism (FIG. 2). The reaction of p-toluidine with FMOC (FIG. 3) contributes to a reduction of ion suppression for closely migrating methylated phospholipids in MSI-NACE-MS in conjunction with back extraction into hexane that was found to be superior to MTBE as organic solvent (FIG. 4). Overall, methylation of acidic phosphoric acid moieties expands the separation window in MSI-NACE-MS by improving the resolution within PL class species as shown in FIG. 1B. Furthermore, cationic methylated PCs migrate with faster migration times and sharper peaks that enhances concentration sensitivity while avoiding ion suppression that occurs predominately within the EOF region due to the co-migration of abundant and electrically neutral plasma lipids (e.g., diacylglycerides, cholesteryl esters etc.). In all cases, a serial injection of seven independent plasma extracts were analyzed rapidly within a single analytical run by MSI-NACE-MS (˜3.5 min/sample) under positive ion mode with full-scan data acquisition. This method also analyzed methylated SM species, which also undergo a distinct mobility and mass shift (+14 Da) as shown in their MS/MS spectra acquired under positive and negative ion mode detection (FIG. 5). SMs have been reported to undergo methylation with a second equivalent on their hydroxyl moiety when using diazomethane, which leads to signal splitting and lower sensitivity gain.⁴² In this case, dimethylated SM species were not detected likely due to the lower reactivity of MTT as compared to diazomethane that requires special safety precautions when handling given its explosive hazards and toxicity.⁴²⁻⁴

### Optimization of FMOC/MTT Phospholipid Derivatization:

MTT was previously introduced as a methylation agent for esterification of carboxylic acids⁴⁷ that allowed for the analysis of acidic metabolites in urine by GC-MS.⁴⁸ Similarly, Furukawa et al.⁴⁹ reported using MTT to methylate oligosaccharides containing sialic acid residues in glycoblotting experiments prior to MALDI-MS analyses. However, this reagent remains unexplored to date with sparse information related to its reaction mechanism and applicability to routine lipidomic analyses. Initial studies were performed to optimize reaction conditions for the formation of methylated PCs as a function of three experimental factors, namely reaction time (0 to 180 min), MTT concentration (50 to 900 mM) and reaction temperature (20 to 100° C.). A maximum yield for methylated PCs was achieved using 450 mM of MTT with a reaction time of 60 min at 60° C. corresponding to an average yield of ˜70%. This apparent reaction yield was lower than first anticipated without the use of FMOC due to ion suppression effects from p-toluidine formed as a by-product when using excess MTT (data not shown). A kinetic study was next performed to determine the minimum reaction time needed when using a two-step chemical derivatization strategy based on FMOC/MTT, where the reaction progress was reflected by a more intense golden/amber hue color as shown in FIG. 2A. Also, FIG. 2B highlights that the reaction yield plateaued at 60 min as shown for 16 representative plasma PCs species analyzed from NIST SRM-1950 when using MSI-NACE-MS. Importantly, the use of FMOC and hexane back extraction alleviated the issues of isobaric lipid interferences and ion suppression effects, resulting in higher and more consistent quantitative reaction yields (90.1±6.4) % as demonstrated in FIG. 2C. In some instances, the use of FMOC nearly doubled the reaction efficiency for certain methylated PCs (e.g., PC 36:5, PC 36:4, PC 40:6) as they only had a ˜45% reaction yield when using MTT alone. The derivatization yield was assessed by taking the ratio of the normalized signal for each underivatized PC prior to and after FMOC/MTT treatment of NIST SRM-1950 human plasma (refer to equation 1) when using a conventional single sample injection format in NACE-MS. This process ensured that native PCs were adequately resolved from the EOF to avoid ion suppression as highlighted for PC 32:1 in FIG. 2D. However, a limitation of the hexane back extraction protocol following FMOC/MTT derivatization was that more polar lipid classes from plasma extracts were not adequately recovered, including shorter chain PCs (<30:0) and lysophosphatidylcholine (lysoPCs). However, most of these polar PC species can be directly analyzed by MSI-NACE-MS under negative ion mode detection without FMOC/MTT derivatization.³⁸ Indeed, plasma lipidomic protocols that rely on more polar organic solvent mixtures for single-phase extraction often suffer from limited recovery and poor solubility for non-polar lipids that prevents their accurate quantification.⁵⁰

### Expanded Lipidome Coverage and Classification Via Mobility Maps:

Similar to the use of collisional cross-section areas for classifying lipid structures as gas-phase ions in IMS,³¹ the electrophoretic mobility represents an intrinsic physicochemical parameter for characterizing ionic lipids in solution by MSI-NACE-MS.³⁸ Zwitter-ionic PC species that migrate close to the EOF under alkaline BGE conditions overlap substantially resulting in a narrow separation window as compared to acidic lipid classes, such as PEs, phosphatidylinositols (PIs), lysophosphatidic acids (LPAs), and free/nonesterified fatty acids (FAs). This scenario was suboptimal for PCs and SMs as it can contribute to false discoveries from isobaric interferences when performing untargeted lipidomics. FIG. 3 (top) highlights that a large mobility shift with improved separation resolution occurred following FMOC/MTT derivatization for two major classes of PLs, namely methylated PCs (n=48) and SMs (n=27). These plasma PLs were annotated based on their sum composition, mass error (<10 ppm) and relative migration times (RMTs) or apparent electrophoretic mobilities (Table 1, 2). Moreover, these cationic phospholipids also satisfied the selection criteria when using temporal signal pattern recognition in MSI-NACE-MS to reject spurious signals and background ions,³⁸ which were also independently verified as consensus plasma lipids in an inter-laboratory harmonization study using NIST SRM-1950.15 In general, methylated SMs migrated with a slower positive mobility than PCs due to differences in their chemical linkage bonding that impacts their PC 32:1 conformational size in solution. Among methylated PC and SM species having similar masses (i.e., PC 32:2≈SM 36:2; O2), the SMs migrated later due to their longer acyl chains resulting in their slower overall electrophoretic mobility in solution. Also, there were characteristic mobility shift patterns evident within both PL sub-classes,³⁸ since a longer fatty acyl backbone (C30-C44) and greater degrees of unsaturation (n=0-8) predictably reduce or increase the apparent mobility for methylated PCs and SMs, respectively as previously shown for various acidic lipids and FAs.^{36,38} The separation resolution of native zwitter-ionic PLs under these conditions was otherwise poor in MSI-NACE-MS as they co-migrate close with the EOF. The steepness of the slope for underivatized PLs reflects their inadequate within-class separation, which are also prone to ion suppression effects. The benefit of methylation of plasma PLs is more clearly illustrated in FIG. 3 (bottom), which compares mobility changes among saturated PCs (including predicted mobility for non-detected PCs via extrapolation), as well as a homologous series of PC 36, PC 38 and PC 40 that demonstrate a linear increase in their positive electrophoretic mobility as a function of higher degrees of unsaturation when using a least-squares linear regression model (R²>0.930). Despite their similar charge state, more highly unsaturated methylated PCs in this case have smaller hydrodynamic sizes in solution than less unsaturated or fully saturated homologues.

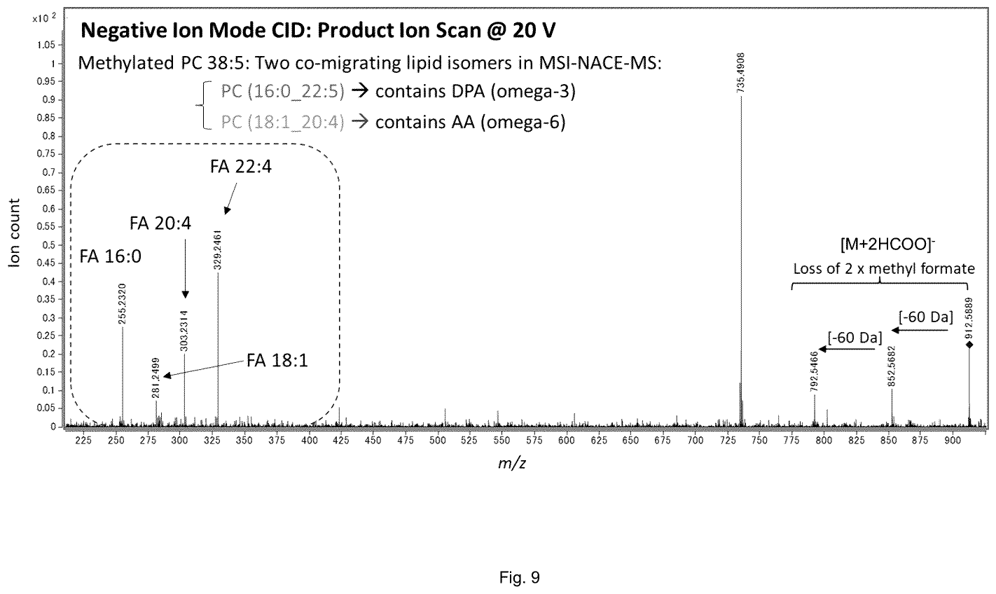

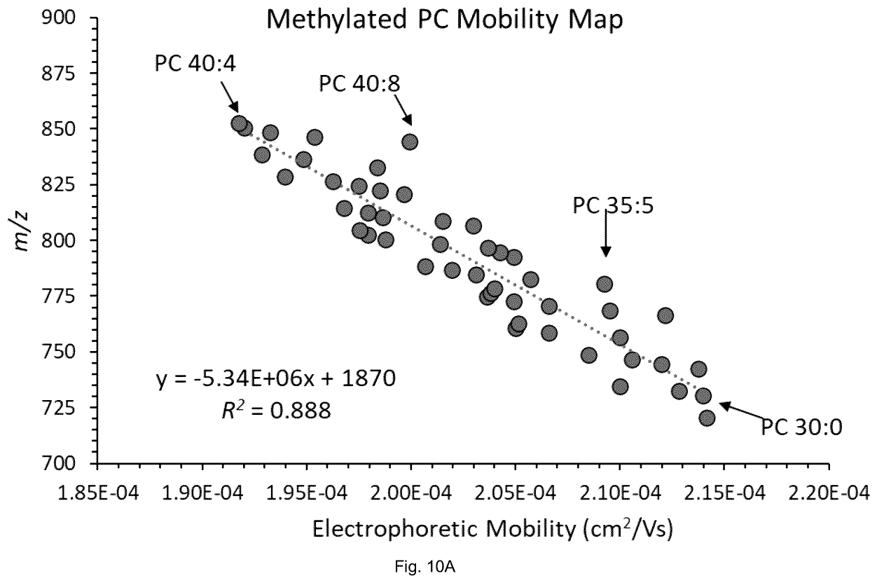

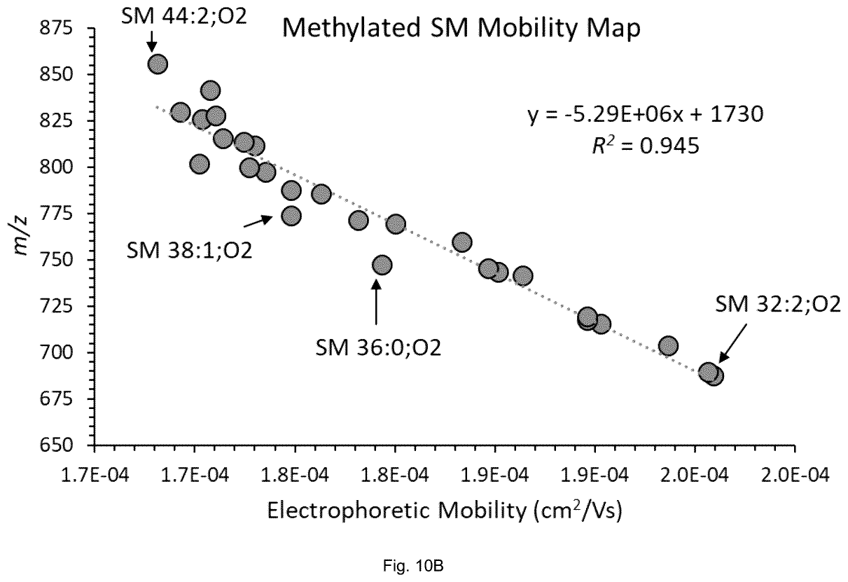

FIG. 4 confirms that the large mobility shift was a result of formation of a cationic phosphate methyl ester as shown in the MS/MS spectra acquired for PC 40:6 under positive and negative ion mode. Annotation of the MS/MS spectra under positive ion mode (at 40 V) for methylated PC 40:6 relative to native PC 40:6 confirmed a diagnostic product ion for its methylated phosphate headgroup (m/z 198.0982) corresponding to a mass shift of m/z 14 as compared to the native PC (m/z 184.0773). Also, annotation of the MS/MS spectra acquired under negative ion mode (at 30 V) confirmed that both methylated PC 40:6 and native PC 40:6 contained a stearic acid (18:0) and docosahexaenoic acid (22:6, DHA) with the latter likely from a sn−2 position when comparing the signal fragment ratio for the two fatty acyl chains. Interestingly, a double formate adduct anion [M+200CH₃]⁻ was detected as the molecular ion for methylated PC 40:6 (PC 18:0_22:6) when acquiring MS/MS spectra in negative ion mode since formic acid was included as an electrolyte in the BGE and sheath liquid solution. This was reflected by a characteristic neutral loss of m/z 60 (methylformate) that occurred twice as compared to only once for native PC 40:6. Moreover, methylated PC 40:6 generated a unique base peak product ion at m/z 761.5081 in negative ion mode corresponding to a neutral loss of methylformate unlike native PC 40:6. However, not all methylated PC isomers from NIST SRM-1950 plasma extracts were comprised of fully resolved species in MSI-NACE-MS as highlighted for methylated PC 38:5 after acquiring MS/MS spectra under negative ion mode (FIG. 9), which comprised a mixture of two co-migrating PL species, namely PC 16:0_22:5 and PC 18:1_20:4. Distinctive MS/MS spectra were also acquired for methylated SM 34:1; O2 under positive and negative ion mode conditions (FIG. 5) that confirmed the same methylated phosphorylcholine head group, but lacked diagnostic fatty acyl chains, which may be better achieved as their lithiated adducts to lower the energy barrier in collision-induced dissociation.⁵¹ Other approaches are needed to confirm the exact stereochemistry of methylated PL molecular species and their potential isomers from human plasma extracts, such as the location of unsaturation and/or geometric configuration when using MS/MS when using ozone-induced dissociation experiments⁵² or photochemical derivatization.⁵³ Nevertheless, mobility plots generated separately for a series of methylated PCs and SMs provide complementary information to deduce the probable chemical structure of plasma PLs and reject potential isobaric candidates as compared to relying on accurate mass alone (FIG. 10-). Overall, MSI-NACE-MS combines the selectivity of HILIC (i.e., polar head group/chemical linkage) and reversed-phase (i.e., total carbon chain length) chromatography, which is optimal for the rapid analysis of ionic classes of lipids from volume or mass-limited samples.³⁸

Characterization of Consensus mPLs from Reference Plasma Sample:

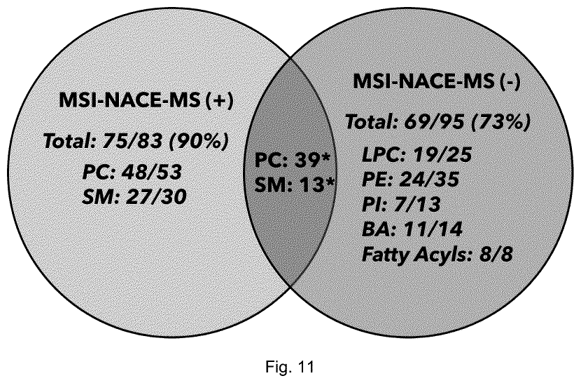

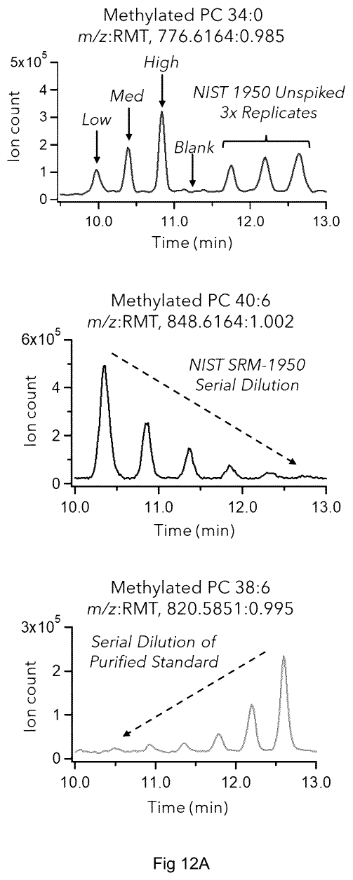

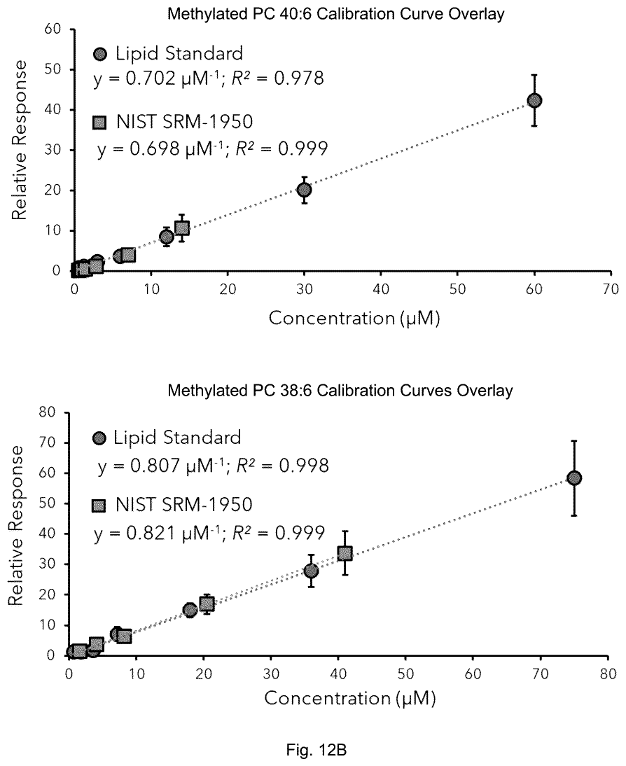

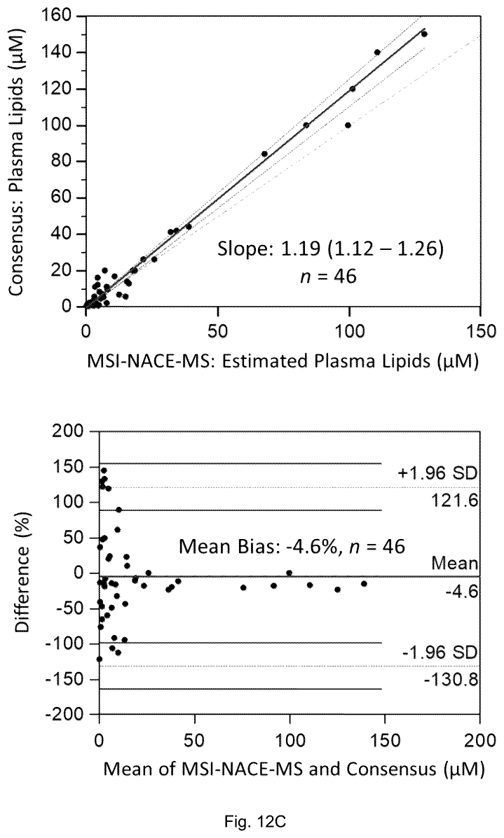

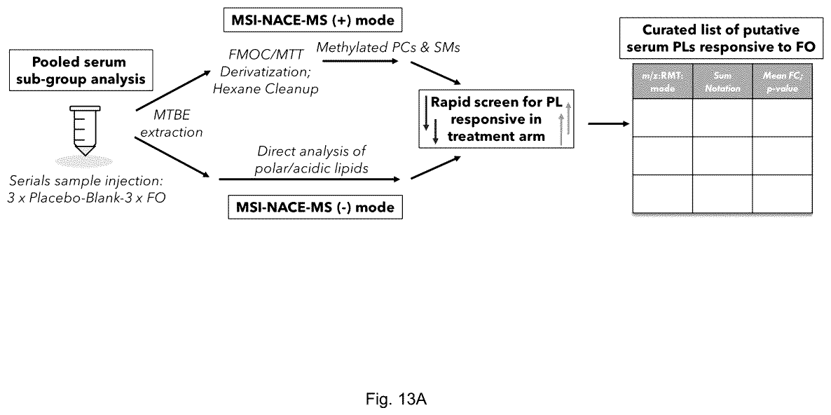

Previously, Bowden et al.¹⁵ reported the use of NIST SRM-1950 as a reference sample when comparing the performance of untargeted lipidomic platforms across 31 international laboratories, each using their own analysis data workflows, LC-MS methodology and hardware/software configuration. Although 1527 unique lipid features were measured quantitatively across all sites, only 339 of these plasma lipids were reported consistently from at least 5 or more laboratories with adequate precision based on a minimum coefficient of dispersion threshold (COD <40%). Next, the two-stage chemical derivatization protocol using MSI-NACE-MS was validated for a panel of methylated PCs and SMs measured consistently from NIST-SRM-1950 plasma extracts as compared to various standardized LC-MS protocols. Overall, 75 plasma PLs reported in the harmonization study were annotated based on their sum composition from NIST SRM-1950 ether extracts in a targeted manner, including 48 PCs and 27 SMs as their cationic phosphate methyl esters (Table 1; Table 2). Overall, MSI-NACE-MS was able to measure 90% of reported consensus PCs (48 out of 53) and SMs (27 out of 30) from NIST SRM-1950, respectively based on the combined PL annotations used by Bowden et al.¹⁵, which also included mass resolvable plasmanyl and plasmenyl species. However, the latter lipid species were confirmed to not be detected in this case. An analysis of acidic lipids from NIST SRM-1950 was also performed when using MSI-NACE-MS under negative ion mode without chemical derivatization to expand lipidome coverage to include more polar classes of acidic lipids under alkaline conditions.³⁸ This also includes LPCs that have a poor recovery after hexane back extraction and PEs that generate isobaric interferences with PCs after methylation if FMOC was not included as a protecting agent. In this case, it was possible to reliably measure 11/14 (79%) bile acids (BAs), 19/25 (76%) of LPCs, but only 24/35 (69%) PE and 7/13 (54%) PI species from the consensus plasma lipids reported by five or more laboratories in Bowden et al.¹⁵ The reduced coverage was likely due to the lower ionization efficiency of polar/acidic lipids under negative ion mode detection in conjunction with the much smaller sample volumes introduced in-capillary (˜10 nL) in MSI-NACE-MS than LC-MS methods. Although only 8 FA species satisfied the selection criteria in the lipidomics harmonization study, MSI-NACE-MS can quantify more than 20 FAs from blood extracts as described elsewhere.^{6,53} FIG. 11 depicts a Venn diagram for consensus PLs from NIST SRM-1950 that were measured by MSI-NACE-MS under both positive and negative ion mode. As expected, a larger fraction (˜50%) of methylated PCs and SMs were measured consistently by MSI-NACE-MS in positive ion relative to negative ion mode without chemical derivatization. This was due to the improved separation resolution and greater ionization response achieved for cationic PCs and SMs following FMOC/MTT derivatization and hexane back extraction. Overall, this work highlights that >150 ionic lipids can be measured in reference human plasma by MSI-NACE-MS under two complementary configurations, including phosphatidylserines (PSs) and PAs that were not reported as consensus plasma lipids from NIST SRM-1950 when using LC-MS methods.¹⁵ For comparison, large-scale CE-MS metabolomic studies using aqueous BGE conditions typically measure <90 polar/hydrophilic metabolites consistently in blood specimens under positive and negative ion mode when using a coaxial sheath liquid flow interface.^{39,54}

### Semi-Quantification of Phospholipids Via Consensus Concentrations in Reference Plasma:

A major analytical challenge in contemporary lipidomic research remains reliable quantification given the lack and/or high costs of lipid standards and matching stable-isotope internal standards. However, a key advantage of MSI-NACE-MS is that ionic lipids migrate with a steady-state mobility under isocratic BGE conditions while using a continuous sheath liquid solution during ionization unlike LC-MS methods that rely on gradient elution for optimal separation performance. Multiplexed separations in MSI-NACE-MS not only improve sample throughput, but also enable versatile serial sample injection configuration to encode mass spectral information temporally within a separation,³⁸ which reduces mass ambiguities when credentialing ionic lipids in an untargeted manner.⁵⁵ FIG. 5A highlights that different serial injection configurations can be designed in MSI-NACE-MS within a single run, such as a spike recovery study for methylated PC 34:0 in NIST SRM-1950 human plasma, a serial dilution of NIST SRM-1950 to estimate the relative response ratio of methylated PC 40:6, and a serial dilution of a lipid standard for methylated PC 38:6 for generation of an external calibration curve. Spike and recovery experiments using four PC lipid standards were also performed at three different concentration levels (low, medium, high) ranging from 1.0 to 20 μM (n=5). In all cases, methylated PCs and SMs were normalized to a single deuterated internal standard given the lack of ion suppression or enhancement effects in MSI-NACE-MS after sample workup. The potential for reliable quantification of methylated PCs was evaluated by comparing relative response factors (i.e., sensitivity) generated from the slopes of calibration curves for each lipid standard with those derived for the same lipid following a serial dilution of NIST SRM-1950 human plasma. In the latter case, consensus (median of mean) PL concentrations reported in a lipidomics harmonization study¹⁵ were used to construct calibration curves. FIG. 5B depicts two representative calibration curve overlays for methylated PC 38:6 and PC 40:6, which highlights good mutual agreement in measured sensitivity (i.e., slope of calibration curve) based on a least-squares linear regression with excellent linearity (R²>0.980). This comparison also confirmed the lack of matrix-induced ion suppression in MSI-NACE-MS given minimal differences (bias<2%) in the apparent sensitivity measured from calibrant standards and directly in reference plasma extracts.

Table 3 summarizes the performance of MSI-NACE-MS for reliable quantification of four representative plasma PCs when using external calibration curves as compared to a serial dilution of NIST SRM-1950. As expected, good accuracy was achieved when quantifying methylated PC 34:0, PC 38:6, and PC 40:6 in both spike-recovery studies, as well as unspiked reference plasma (mean bias<10%) when using calibration curves by MSI-NACE-MS when compared to untargeted LC-MS methods.¹⁵ Slightly higher bias (<25%) was found for PC 38:6 and PC 40:6 concentrations in NIST SRM-1950 when compared to a targeted shotgun (separation-free) lipidomic inter-laboratory comparison study by DI-MS/MS using a commercial lipid kit under standardized operating conditions.¹⁷ The latter discrepancy may arise due to isobaric interferences when high efficiency separations are not used in lipidomic analyses. Overall, poor accuracy (mean bias ˜−50%) was noted primarily for PC 30:0 after hexane sample cleanup since this procedure favors a quantitative recovery of more lipophilic PLs having longer total carbon acyl chain lengths. An alternative strategy for semi-quantitative estimation of other plasma PLs lacking chemical standards was also explored via response factors derived from the serial dilution of NIST SRM-1950 when using the median of mean consensus lipid concentrations reported by Bowden et al.¹⁵ As expected, this strategy was better suited to more abundant plasma PLs (>10 mM) given the serial dilution process unlike lipid standards that permitted PL quantification over a wider linear dynamic range (FIG. 5B). Overall, 21 plasma PC (n=14) and SM (n=7) species were measured in at least 4 concentration levels with adequate precision (CV<20%) and linearity (mean R²=0.987) as summarized in Table 4. This in turn was used to estimate the response factors and corresponding concentrations for 46 annotated plasma PLs (>0.5 mM), including 19 PCs and 27 SMs (Table 5). In cases where a direct measurement of a response factor was not feasible by MSI-NACE-MS due to inadequate dynamic range, the closest PL analog in terms of mass and lipid class from Table 4 was used as a surrogate to estimate its response factor. FIG. 4C demonstrates that this approach generally resulted in a good mutual agreement when estimating the concentration for most plasma PLs by MSI-NACE-MS as compared to their consensus concentrations by several LC-MS methods as reflected by a slope of 1.19 (95% CI: 1.12-1.26) and a mean bias of-6.9% over a 500-fold dynamic range (0.5 to 200 mM). Yet, greater bias and variability was evident for lower abundance PLs (<5 mM) as response factors were more difficult to reliably assess in MSI-NACE-MS following serial dilution of NIST SRM-1950 resulting in the reliance of non-matching PL surrogate species. For instance, the average bias was acceptable at-9.7% for most plasma PLs (n=27) having reported consensus concentrations >5.0 mM in contrast to a larger average bias of 104% for PLs<5.0 mM (n=17). The latter group of PLs comprised mostly lower abundance SMs and PCs that relied on surrogate PLs to estimate their response factor with greater uncertainty (Table 5). Further work is needed to further evaluate the quantitative accuracy and long-term analytical performance of MSI-NACE-MS for plasma PLs when using FMOC/MTT derivatization.

Nevertheless, this approach offers a higher throughput approach for quantitative lipidomic analyses even in cases when standards are not available, which was recently applied to identify two specific circulating PCs as surrogate biomarkers of the omega-3 index following high-dose fish oil, docosahexaenoic acid or eicosapentaenoic acid supplementation (see Example 2).⁵⁵ In summary, expanded lipidome coverage was achieved in MSI-NACE-MS when using a two-step pre-column chemical derivatization strategy to convert zwitter-ionic PLs into their corresponding cationic methyl phosphate esters. This labeling procedure is quantitative and more convenient to use than diazomethane for PL methylation, which results in improved separation performance and ionization efficiency. Overall, 75 cationic PCs and SMs were characterized from reference human plasma with adequate precision when using MSI-NACE-MS following FMOC/MTT derivatization and hexane back extraction as compared to an international lipidomic harmonization study. Additionally, more than 69 other acidic and polar PLs from NIST SRM-1950 plasma extracts can also be measured by MSI-NACE-MS under negative ion mode without chemical derivatization, not including polar lipid classes poorly retained in reversed-phase LC-MS (e.g., PAs, PSs, FAs). This strategy greatly expands conventional CE-MS metabolomic protocols that rely on aqueous buffer systems and thus have been limited to the analysis of hydrophilic/polar metabolites. Lipid annotation and structural classification was also supported based on predictable trends in the electrophoretic mobility for methylated PCs and SMs that are dependent on polar head group/chemical linkage, total fatty acyl chain length and degrees of unsaturation. Advantages of MSI-NACE-MS include greater throughput and minimal ion suppression effects that allows for unique data workflows for data acquisition and lipid authentication in comparison to other separation methods that utilize single sample injections. MSI-NACE-MS is also more amenable to standardization since it operates using only a bare-fused silica capillary under an isocratic nonaqueous buffer system unlike LC-MS that rely on different column types and gradient elution programs when using reversed-phase and HILIC separations. However, MSI-NACE-MS with a coaxial sheath liquid interface suffers from higher detection limits and lower concentration sensitivity for ionic lipids as compared to LC-MS protocols due to the smaller sample volume introduced on-capillary. Also, electrically neutral lipid classes are not resolved or reliably measured even after methylation, such as diacylglycerides and cholesteryl esters.

### Conclusion:

In this work, a two-step chemical derivatization strategy was introduced using FMOC/MTT for the methylation of zwitter-ionic PLs to expand lipid profiling coverage by MSI-NACE-MS under positive ion mode conditions. FMOC was used as a compatible protecting agent to prevent generation of PE isobaric species to PCs that also reduced ion suppression effects from excess MTT by-products prior to hexane back extraction. The efficacy of this reaction was optimized to generate quantitative yields of 75 cationic methylated PCs and SMs verified in reference human plasma when using MSI-NACE-MS, which comprised 90% of consensus plasma lipids within these two classes as reported in an international lipidomics harmonization study. Overall, PL methylation resulted in improved separation resolution, faster analysis times, reduced ion suppression while allowing for better lipid structural classification based on changes in their electrophoretic mobility. This method is optimal for lipidomic studies requiring higher sample throughput and lower operating costs with stringent quality control, while consuming minimal volumes of sample and organic solvent. Complementary analysis of other polar or acidic lipid classes can be achieved by their direct analysis using MSI-NACE-MS under negative ion mode without chemical derivatization. Good precision and accuracy was also demonstrated when quantifying methylated PCs and SMs in reference plasma samples, including the potential for use of serial dilution of NIST SRM-1950 to estimate relative response factors for lipids lacking chemical standards provided they are present at concentrations >5 mM. This methylation strategy offers a practical alternative to diazomethane for improved lipid analysis when using other MS instrumental platforms without excessive hazards and safety precautions, including direct infusion-MS, ion mobility-MS and LC-MS/MS methods. Although the applicability of this two-tiered derivatization scheme s demonstrated on phosphatidylcholine (PC) and sphingomyelin (SM) using MSI-NACE-MS, other lipid species containing phosphoric acid moieties that exist in biological samples are subject to separation and ionization enhancement using derivatization. This can be especially useful when trying to profile polar lipids using conventional nontargeted lipidomics protocols using ion mobility and/or chromatographic separations coupled to high resolution MS. For example, methylation using trimethylsilyl-diazomethane has been demonstrated to improve separation efficiency and responsiveness of various polar lipids by supercritical fluid chromatography/tandem mass spectrometry (SFC/MS/MS) (134), and more recently, even enhancing resolution of phosphoinositide regioisomers (135). Additionally, a methylation approach was shown to be effective for profiling fatty acyl-coenzyme As (acyl-CoAs) (137) by improving peak shape and reducing carry over effects. The use of FMOC and MTT presents as an alternative strategy for methylation of polar phosphoric acid containing lipid species to enhance column retention, resolution and ionization response for larger scale, routine analysis with considerably lower hazards than diazomethane.

### Example 2

Evidence-based nutritional policies are urgently needed given an alarming increase in obesity and cardiometabolic disease burden worldwide (56). Public health guidelines have historically focused on lowering dietary fat intake (e.g., cholesterol, saturated fats) as a purported ‘heart healthy’ diet (57) rather than assessing overall diet quality (58). For instance, there is widespread deficiency of omega-3 long-chain polyunsaturated fatty acids (n3-LCPUFAs) as it comprises only a small fraction of total fats consumed in contemporary Western diets (59) since endogenous synthesis of this important class of fatty acid (FA) is low (60). For these reasons, the American Heart Association Nutrition Committee recommends the consumption of oily fish/seafood as a marine source of dietary n3-LCPUFA up to twice a week to reduce cardiovascular disease risk (61). Unlike saturated or monounsaturated FAs, humans are unable to synthesize sufficient amounts of n3-LCPUFAs enriched within the cellular membrane of certain tissues/organs (e.g., retina, brain, heart), including docosahexaenoic acid (DHA, 22:6) and eicosapentaenoic acid (EPA, 20:5) (62). Omega-3 FA nutrition impacts membrane composition and cellular function while also modulating inflammatory processes, such as the formation of resolvins and anti-inflammatory lipid mediators (63). Optimal intake of n3-LCPUFAs may also improve skeletal muscle function in older persons by enhancing amino acid-stimulated muscle protein synthesis rates, and mitochondrial respiration kinetics (64).

Although dietary sources of n3-LCPUFAs are derived primarily from marine sources, the content of DHA and EPA in commonly consumed wild and farmed fish species varies widely (65). Alternatively, commercial fish oil (FO) dietary supplements offer a way to ensure adequate omega-3 FA nutrition together with emerging microalgae sources (66), and prescription EPA and/or DHA products (67). Yet, there have been conflicting results of n3-LCPUFAs in clinical trials in terms of their efficacy for cardiovascular disease protection (68). This outcome likely stems from inadequate dosage (<2 g/day) using impure formulations that do not target high-risk patients with hypertriglyceridemia and other comorbidities having a low baseline O3I status (69). For instance, high-dose prescription (4 g/day) icosapent ethyl treatment has been reported to reduce cardiovascular events in current and former smokers to levels similar to never smokers (70). However, prescription, supplemental and/or dietary intake of n3-LCPUFAs do not address excessive consumption of omega-6 FAs prevalent in processed foods (71), or differences in fatty acid desaturase activity (72) that contribute to variations in treatment response.

### Methods

### Study Designs, Participants and Omega-3 FA Supplementation Trials:

Both human n3-LCPUFA supplementation trials in this study obtained signed informed consent from all participants and abided by ethical principles of the Declaration of Helsinki. In the first discovery cohort, fasting serum samples were collected from participants in a randomized, double-blinded, placebo-controlled intervention study that investigated the effects of FO supplementation on attenuating skeletal muscle disuse atrophy following leg immobilization (84). This clinical trial was registered at the US National Library of Medicine (https://clinicaltrials.gov/) as NCT03059836, and approved by the Hamilton Integrated Research Ethics Board. Briefly, this study comprised a cohort of healthy young women with a mean age of 22 years (range: 19-31 years) and BMI of 24 kg/m² (range: 18-26 kg/m²) recruited locally from the Hamilton area. All participants received either a high-dose FO (3.0 g EPA and 2.0 g DHA daily; n=9) or a placebo control based on an isoenergetic and volume equivalent sunflower oil (SO) daily (n=9). Repeat fasting serum samples were collected from participants at baseline and after 28, 42 and 56 days of the intervention. All serum samples were then stored frozen at −80° C. Further details on blood collection, participant selection and exclusion criteria, and erythrocyte PL omega-3 FA analysis for O3I determination are described elsewhere (84). Briefly, lipids were extracted from red blood cells using the Folch method (90) in chloroform-methanol (2:1 vol.) containing butylated hydroxytoluene (BHT, 0.01% vol.) as an antioxidant and heptadecanoic acid as an internal standard. Thin-layer chromatography silica plates isolated PL fractions (Silica Gel 60, 0.22 mm; Merck, Kenilworth, NJ, USA) using heptane: isopropylether: acetic acid (60:40:3 vol.) as the elution solvent. Gel bands were scraped off the plate and transferred into screw cap tubes for transmethylation with BF₃ in methanol. Fatty acid methyl esters (FAMEs) were then dissolved in hexane and analyzed using a Hewlett-Packard 5890 Series II GC with flame-ionization detection while using a Varian CP-SIL capillary column (100 m, internal diameter of 0.25 mm) (Palo Alto, CA, USA). These measurements were then used to calculate the O3I by taking the sum of quantified EPA and DHA relative to the total of 17 saturated, monosaturated and polyunsaturated FAs in fasting serum samples.

In the second validation cohort, fasting plasma samples were collected from participants in a randomized, double-blinded, multi-arm, placebo-controlled parallel group trial comparing the effects of supplementing using either ˜3 g/day EPA, DHA, or olive oil (OO) over a 90-day period (85). This study was approved by the Research Ethics Board at the University of Guelph. Participant characteristics (sex, age, BMI etc.) and blood draws for O3I assessment were obtained for all participants (n=83) on study day visits. Purified EPA (KD-PUR EPA700TG) and DHA (KD-PUR DHA700TG) oils, as well as OO, were obtained from KD Pharma (Bexbach, Germany) with EPA and DHA in their triglyceride forms. The FA content of these supplements was previously reported to be 75.7%±0.01% for oleic acid (18:1) in the OO supplement, 74.7%±0.09% EPA and 0.55%±0.01% DHA in the EPA supplement, and 72.3%±1.3% DHA and 1.05+0.11% EPA in the DHA supplement (91). All capsules contained 0.20% vol. tocopherol to prevent oxidation of polyunsaturated lipids. Exclusion criteria included use of FO supplements within the previous 3 months, >2 servings of fish/seafood or other omega-3 FA-rich products per week, prescribed medication use (except oral contraceptives), current smoking and history of cardiovascular disease. Participants were assigned via block randomization with stratification by sex to one of three treatment arms, namely OO supplement (n=27), EPA supplement (n=28) and DHA supplement (n=28). Participants were instructed to maintain regular exercise and dietary habits throughout the study. After overnight fasts, participants were subject to blood sampling at the Human Nutraceutical Research Unit at the University of Guelph before (baseline) and after (endpoint) of the 90-day intervention period. Blood was collected into EDTA-treated vacutainers was used to isolate plasma and erythrocytes. Samples were separated by centrifugation at 700×g at 4° C. for 15 min. A similar protocol was performed for FAME analysis from erythrocyte PL extracts following fractionation and saponification using GC-FID with normalization to heptadecanoic acid as internal standard (92). In this case, the O3I was calculated by taking the sum of EPA and DHA relative to the sum of 15 saturated, monosaturated and polyunsaturated FAs in fasting plasma samples.

### Sample Workup, Extraction and Derivatization Procedure for MSI-NACE-MS:

Fasting serum and plasma samples were subject to a two-step chemical derivatization protocol using 9-fluorenylmethyoxycarbonyl chloride (FMOC) and 3-methyl-1-p-tolyltriazene (FMOC/MTT) as described in example 1. This reaction was introduced as a more convenient alternative to diazomethane to improve separation resolution and ionization efficiency by converting zwitter-ionic PL species that co-migrate close to the electroosmotic flow (EOF) into methylated phosphatidylcholines (PCs) and sphingomyelins (SMs) with a permanent positive charge. Briefly, in a glass sample vial, a 50 μL aliquot of serum/plasma sample was subject to a methyl-tert-butyl ether (MTBE) extraction, where 100 μL of methanol with 0.01% vol of BHT as antioxidant, and PC 16:0[D62] as internal standard were first added, and samples then mixed to induce protein precipitation. Next, 250 μL of MTBE was added and mixed prior to adding 100 μL of deionized water to induce phase separation. Samples were then centrifuged at 4000 g at 4° C. where then 200 μL of the organic layer was transferred into a new glass vial and dried down. Next, 100 μL of 0.85 mmol/L FMOC in chloroform containing PC 18:0[D70] as a second internal standard was added to dried serum/plasma extracts and mixed for 5 min at room temperature before drying down again. Next, 50 μL of MTBE containing 450 mmol/L of MTT was added to the glass vial with the lid sealed with Teflon tape. This vessel was then heated to 60° C. for 60 min. Once the reaction was complete, the solution was dried down and then subject to a back extraction, where 100 μL of methanol was added, followed by 250 μL of hexane and then 200 μL of deionized water before centrifuging for 10 min at 4000 g at 4° C. Then, 200 μL of the upper hexane layer was transferred out and dried down. Once completely dried, all samples were subsequently reconstituted in 50 μL containing acetonitrile/isopropanol/water (70:20:10 vol.) with 10 mmol/L ammonium formate and benzyltriethylammonium (BTA) chloride as a third internal standard. All three internal standards had a final concentration of 5.0 μmol/L in the final plasma/serum extract, where PC 16:0[D62] was used for data normalization of methylated PCs to improve method precision based on their relative peak areas (RPA) and relative migration times (RMT). Overall, derivatization yields of about 90% was achieved for quantitative analysis of methylated PCs by MSI-NACE-MS using a reference human plasma sample (87; as described in Example 1).

In order to expand overall lipidome coverage, lipid ether extracts were also analyzed directly without methylation, namely acidic/polar PL classes, including lysophosphatidylcholines (LPCs), phosphatidylethanolamines (PEs), lysophosphatidylethanolamines (LPEs), phosphatidylinositols (PIs) and NEFAs when using MSI-NACE-MS under negative ion mode as described elsewhere (88) and in example 1. Briefly, a 50 μL aliquot was first subjected to MTBE extraction where 100 μL of MeOH containing 0.01% vol. BHT was added to samples containing deuterated myristic acid, FA 14:0[D27] as an internal standard. Following rigorous shaking, phase separation induced by adding water, where samples were centrifuged to sediment protein at 4000 g at 4° C. for 30 min. The formation of a biphasic solution allowed for the top, lipid-rich ether layer to be extracted at a fixed volume (200 μL), where it was then dried under a gentle stream of nitrogen gas at room temperature. The dried extracts were then concentrated 2-fold after reconstitution in 25 μL acetonitrile-isopropanol-water (70:20:10 vol) containing 10 mM ammonium acetate and 50 μmol/L of deuterated stearic acid, FA 18:0[D35] as a second internal standard. However, FA 14:0[D27] was used for data normalization of acidic lipids to improve method precision.

### Untargeted and Targeted Lipidomics of Serum/Plasma Ether Extracts:

An Agilent 6230 TOF mass spectrometer equipped with a coaxial sheath liquid ESI ionization source was used with an Agilent G7100A capillary electrophoresis (CE) unit for all experiments (Agilent Technologies Inc.). To supply a sheath liquid during electrophoretic separations, an Agilent 1260 Infinity isocratic pump delivered a solution containing 80% vol. methanol with 0.1% vol. formic acid at a flow rate of 10 μL/min into the sprayer. All separations were performed using bare fused-silica capillaries with an internal diameter of 50 μm, outer diameter of 360 μm and total length of 110 cm (Polymicro Technologies Inc.). Electrophoretic separations were performed with an applied voltage of 30 kV with the capillary cartridge set at 25° C. while using an isocratic pressure of 10 mbar (1 kPa). The background electrolyte (BGE) was 35 mmol/L ammonium formate in 70% vol. acetonitrile, 15% vol. methanol, and 5% vol. isopropanol with an apparent pH of 2.3 that was adjusted by the addition of formic acid. Derivatized ether extracts were injected hydrodynamically at 50 mbar (5 kPa) alternating between 5 s for each sample plug and 40 s for the background electrolyte spacer plug to total seven discrete samples that were analyzed within 30 min for a single experimental run. Repeat QC samples were created by pooling samples from each study cohort, which were then introduced in a randomized position for each MSI-NACE-MS run to assess the technical precision of the method in both FO (n=13) and EPA or DHA (n=29) supplementation trials. All methylated lipid extracts were analyzed in positive ion mode acquisition with a V_{cap} at 3500V with full-scan data acquisition over the range of (m/z 50-1700). Acidic lipids without derivatization were analyzed directly by MSI-NACE-MS under negative ion mode, which was performed only for the pooled sub-group analysis in the discovery FO trial. This instrumental configuration used a sheath liquid of 80% vol. methanol with 0.5% vol. ammonium hydroxide delivered at a flow rate of 10 μL/min using a CE-MS coaxial sheath liquid interface kit. The separations were performed on the same bare fused-silica capillaries with internal diameter of 50 μm, outer diameter of 360 μm and total length of 95 cm. The applied voltage was set to 30 kV at 25° C. for CE separations while applying an isocratic pressure of 20 mbar (2 kPa). The BGE consisted of 35 mM ammonium acetate in 70% vol. acetonitrile, 15% vol. methanol and 5% vol. isopropanol with an apparent pH of 9.5 that was adjusted using the addition of 12% vol ammonium hydroxide. These underivatized serum ether extracts from the FO discovery trial were injected hydrodynamically at 50 mbar (5 kPa) alternating between 5 s for each sample and 40 s for the BGE spacer for a total of seven discrete samples that were analyzed within a 30 min run (87,88,93). The TOF was operated in negative ion mode acquisition with V_{cap} at 3500 V for full-scan data acquisition over the range of (m/z 50-1700).

Overall, untargeted lipid profiling was performed on pooled serum extracts in a sub-group analysis of participants from the FO intervention trial when using MSI-NACE-MS under positive and negative ion modes. This was followed by a targeted lipidomic analysis and subsequent validation of lead candidate PC biomarkers responsive to n3-LCPUFA supplementation in both FO, and EPA or DHA only placebo-controlled trials when using MSI-NACE-MS under positive ion mode following FMOC/MTT derivatization. Structural elucidation of putative PC biomarkers of the O3I were performed by collision-induced dissociation experiments when using a single injection format in CE coupled to a 6550 quadrupole-time of flight-mass spectrometer system (Agilent Technologies Inc.) at different collision energies under positive and negative ion mode as described elsewhere (87,88,93). Access to a purified reference standard for PC 16:0/22:6 (Toronto Research Chemicals, Toronto, ON) was available to confirm the likely molecular structure of PC 38:6 after spiking in pooled plasma (i.e., co-migration) together with a comparison of the relative intensity of fatty acid product ions using MS/MS under negative ion mode. However, lack of access to other lipid standards, including PC 16:0/20:5 and potential positional isomers (e.g., PC 22:6/16:0) prevented the reporting of definitive lipid molecular structures for these lipids in this study. Further details on the methodology used in this study is summarized in a reporting checklist from the Lipid Standards Initiative (https://doi.org/10.5281/zenodo.8339260).

### Data Processing and Statistical Analysis:

All MSI-NACE-MS data was analyzed using Agilent MassHunter Workstation Software (Qualitative Analysis Version 10.0, Agilent Technologies, 2012). All molecular features were extracted in profile mode within a 10 ppm mass window where derivatized lipids were annotated based on their characteristic m/z corresponding to their molecular ion and relative migration time (RMT). The manually integrated peak areas obtained from the extracted ion electropherograms were normalized to PC 16:0[D62] (positive ion mode) or FA 14:0[D27] (negative ion mode) to determine relative peak areas (RPAs) and RMT for serum/plasma lipids. Extracted ion electropherograms were integrated after smoothing using a quadratic/cubic Savitzky-Golay filter (7 points). Absolute concentrations reported for select PLs were estimated based on a serial dilution of NIST SRM-1950 human plasma when using MSI-NACE-MS as described in example 1 based on consensus concentrations reported in a multi-center lipidomics harmonization study (94). However, PC 38:6 was quantified directly using an external calibration curve normalized to PC 16:0[D62], whereas the response factor for PC 36:5 was estimated using a higher abundance surrogate lipid, PC 36:4 needed to attain adequate linear dynamic range after serial dilution of NIST-SRM 1950 human plasma as described in example 1. Least-squares linear regression and correlation plots were performed using Excel (Microsoft Office). Visualization of data, heat maps, and unsupervised principal component analysis (PCA) were performed using MetaboAnalyst version 5.0 (95). Normality tests and nonparametric statistical analysis was performed using IBM SPSS version 23 (IBM), whereas MedCalc version 12.5.0 (MedCalc Software) was used to generate boxplots and control charts with exception of trajectory box plots (R Foundation for Statistical Computing). A two-way between and within mixed-model ANOVA (treatment×time) was used for assessing the impact of high-dose FO supplementation at three times points as compared to baseline. For the study involving DHA or EPA supplementation relative to OO as placebo, a Wilcoxon signed ranked test was performed to evaluate treatment effects after confirming non-normally distributed data. A Pearson correlation analysis was used to evaluate the association between lead candidate PCs in serum or plasma extracts as compared to O3I based on erythrocyte membrane PLs.

### Results and Discussion

### Sub-group Analysis for Identifying Serum PLs Responsive to FO Intake:

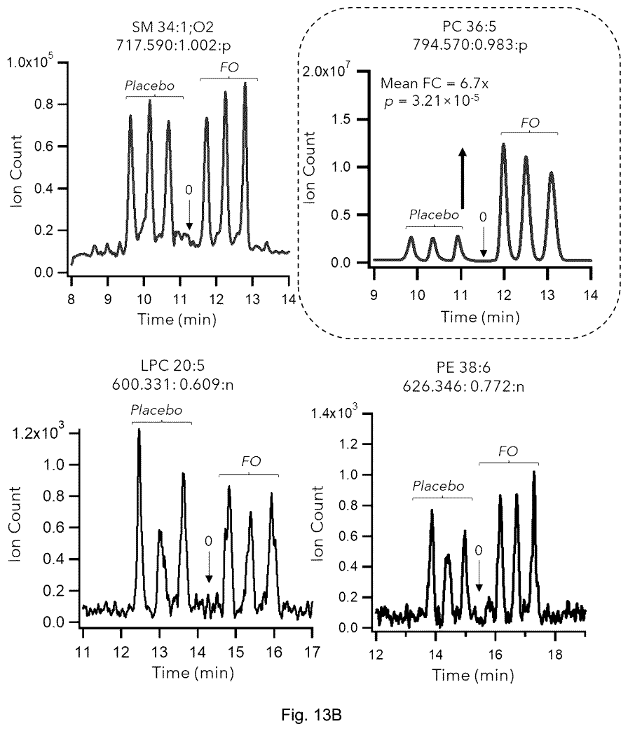

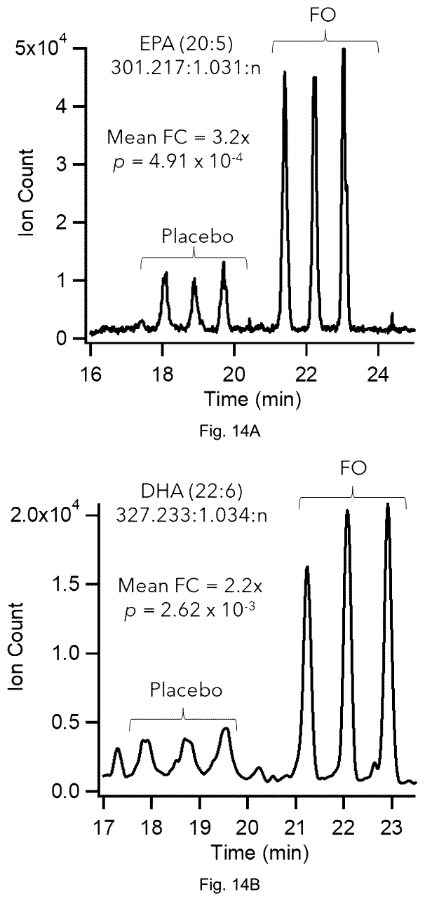

An untargeted screen for serum PLs associated with n3-LCPUFA supplementation was initially performed based on an analysis of pooled serum extracts from all participants in the placebo/baseline as compared to the FO treatment arm (EPA, 3 g/day+DHA, 2 g/day). These two sub-groups of samples were analyzed in triplicate with a blank extract to rapidly identify differentiating PL species following high-dose FO ingestion using two complementary MSI-NACE-MS configurations as shown in FIG. 13A. This strategy takes advantage of a serial injection of 7 serum extracts within a single analytical run by MSI-NACE-MS in positive or negative ion mode when using temporal signal pattern recognition (96,97), and enables reliable credentialing of lipid features responsive to FO ingestion after rejecting spurious signals, background ions and a majority of non-responsive PLs. Overall, serum PCs and SMs as their cationic methylphosphate esters were preferentially analyzed by MSI-NACE-MS under positive ion mode after FMOC/MTT derivatization, whereas electrically neutral lipid classes (e.g., triacylglycerides, cholesterol esters) co-migrate with the EOF as descried in example 1. This two-step chemical derivatization procedure relies on MTT as a less hazardous methylating reagent to diazomethane avoiding the need for blast shields and personal protective equipment (98). However, FMOC was first added prior to MTT to protect certain PLs having reactive primary amine head groups (e.g., PEs) thereby avoiding the generation of permethylated isobaric interferences to analogous PCs as described in example 1. Otherwise, direct analysis of PEs and other acidic/polar lipid classes (e.g., PSs, PAs, LPCs) that did not benefit from methylation or had a poor recovery in hexane was performed by MSI-NACE-MS under negative ion mode conditions to expand overall lipidome coverage (88,89) as described in example 1.

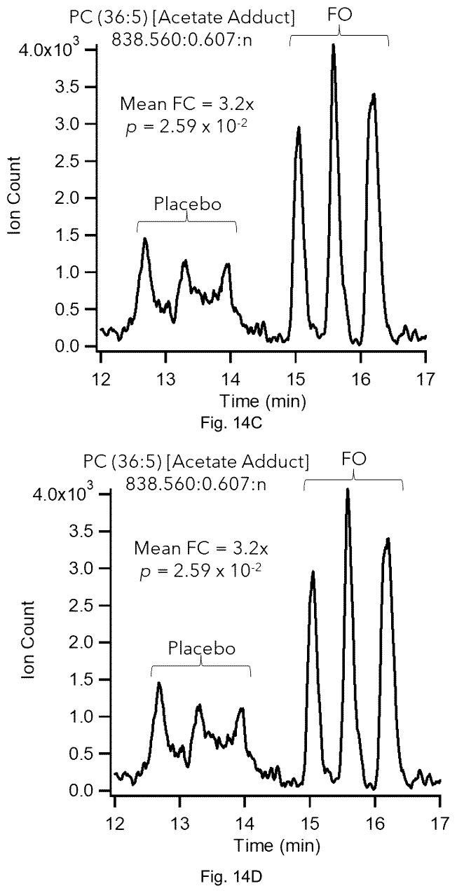

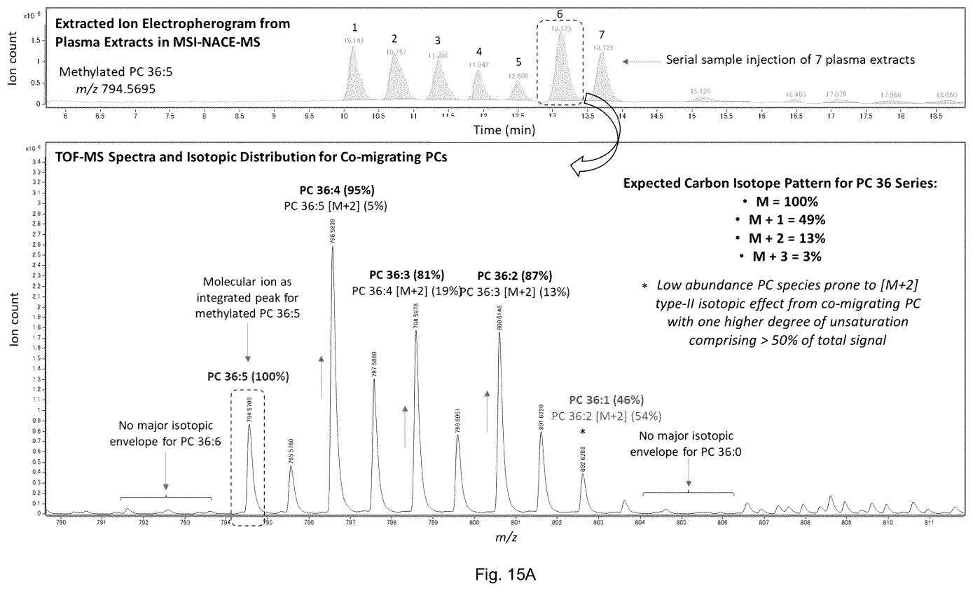

For example, FIG. 13B illustrates four representative PL classes from pooled sub-groups of serum extracts annotated by their accurate mass, relative migration time, ionization mode (m/z: RMT: p or n), and their sum composition, including SM 34:1; O2, PC 36:5, LPC 20:5, and PE 38:6. Importantly, these serum derived lipids were not prone to sample carry-over effects when using serial sample injections in MSI-NACE-MS as demonstrated by a blank extract analyzed within the same run. As expected, serum PLs with only a single degree of unsaturation, such as SM 34:1; O2 did not exhibit a change in response following FO supplementation as compared to baseline/placebo. Two other PL classes measured directly from serum extracts by MSI-NACE-MS under negative ion mode, including a putative EPA-containing LPC 20:5, and a DHA-containing PE (PE 38:6), also did not change (p>0.05) following FO intake. In contrast, PC 36:5 exhibited a striking 10-fold change (FC) average increase from baseline in response to FO supplementation. Similarly, underivatized PC 38:6 and PC 36:5 measured directly under negative ion mode were independently demonstrated to undergo a similar response increase following FO supplementation (FIG. 14). As zwitter-ionic PCs migrate close to the EOF under these conditions, resolution was poor, and their ion responses were prone to matrix-induced ion suppression that lowered overall sensitivity. For these reasons, a quantitative methylation reaction was applied as a charge-switching derivatization strategy in lipidomics to improve their separation resolution and detectability when using MSI-NACE-MS under positive ion mode as described in example 1. This approach also reduces isobaric interferences among distinct lipid classes based on differences in their apparent electrophoretic mobility, such as methylated SMs and PCs as descried in example 1. Other DHA-containing PCs, such as PC 38:6, exhibited a more modest increase after FO intake, as well as EPA and DHA as their NEFAs (86,87). Overall, only six PC species likely containing DHA and EPA fatty acyls chains from a total of 84 annotated ionic lipids (Table 7) were classified as putative lipid biomarkers that increased following FO ingestion together with their NEFAs (p<0.05) under fasting conditions.

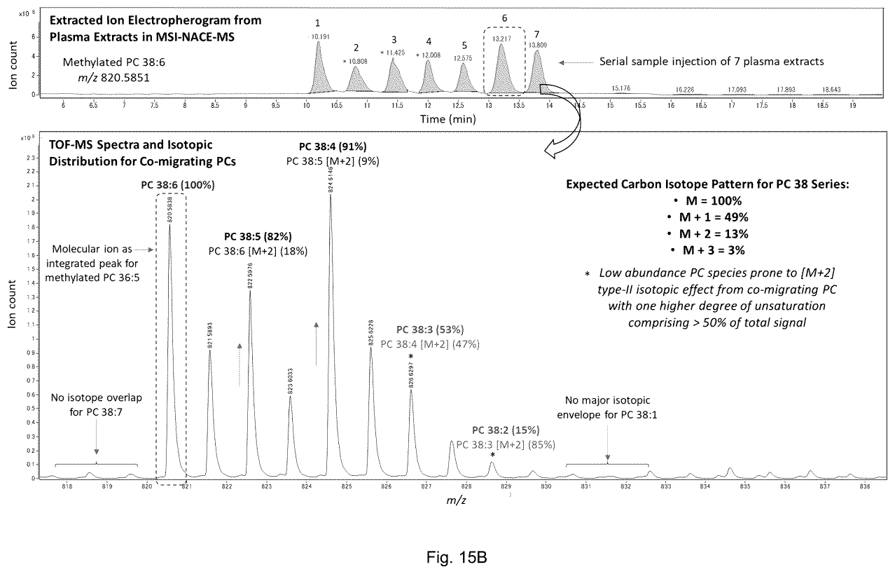

However, other ionic PL classes containing likely EPA or DHA (e.g., LPCs, PIs, PEs, LPEs etc.) were not found to be responsive to FO supplementation in this study. Also, certain serum PLs may comprise unresolved mixtures of isomers or isobars that lack specificity (e.g., PC 38:5), whereas other NEFAs derived from less abundant n3-LCPUFA in FO did not respond to supplementation, such as docasapentaenoic acid (DPA, 22:5). Type-2 isotopic effects were also not a significant problem to correct for as most co-migrating lipid isotopomers, notably for PC 35:5 and PC 38:6 lacked homologous PCs having an additional double bond (FIG. 15). As a result, the focus was on data integration of methylated serum PCs analyzed by MSI-NACE-MS under positive ion mode detection, notably top-ranked candidate biomarkers of omega-3 FA nutrition identified by this untargeted lipidomics screen involving pooled sub-groups of participants prior to and following high-dose FO supplementation.

Validation of Serum PC Panels Associated with High-Dose FO Supplementation:

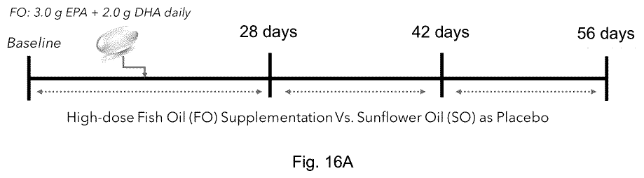

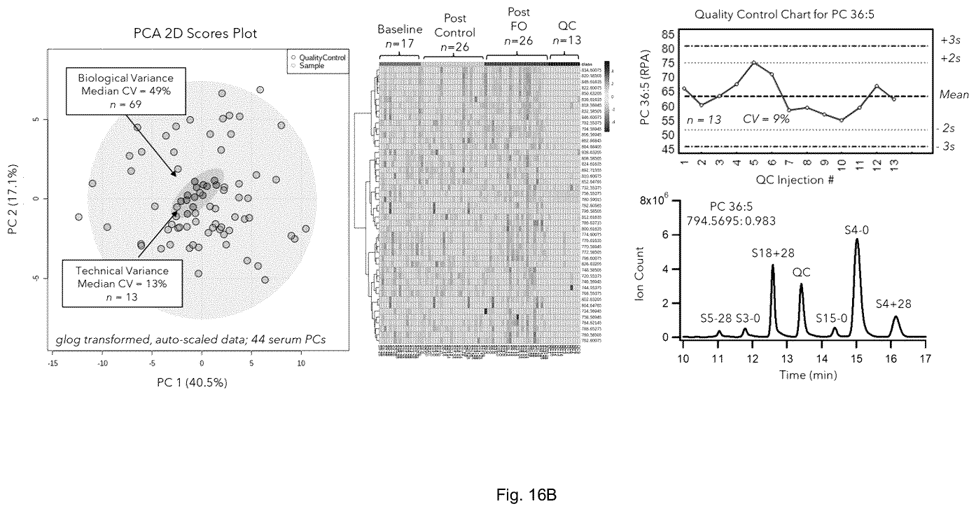

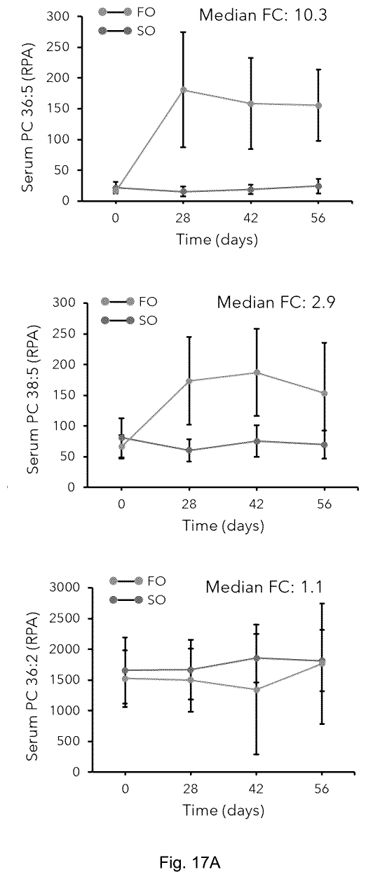

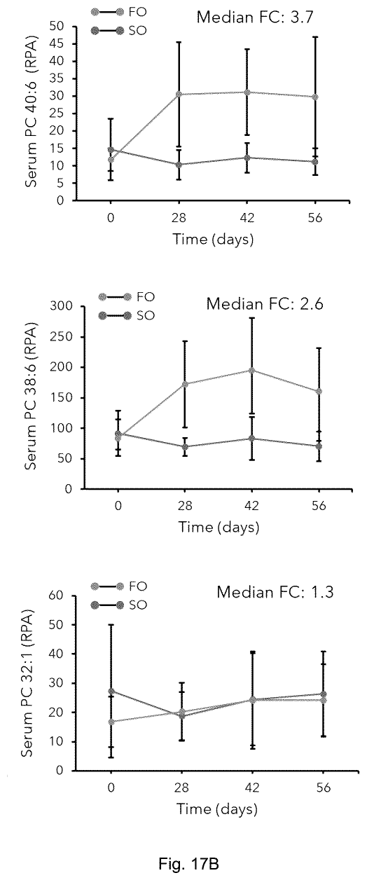

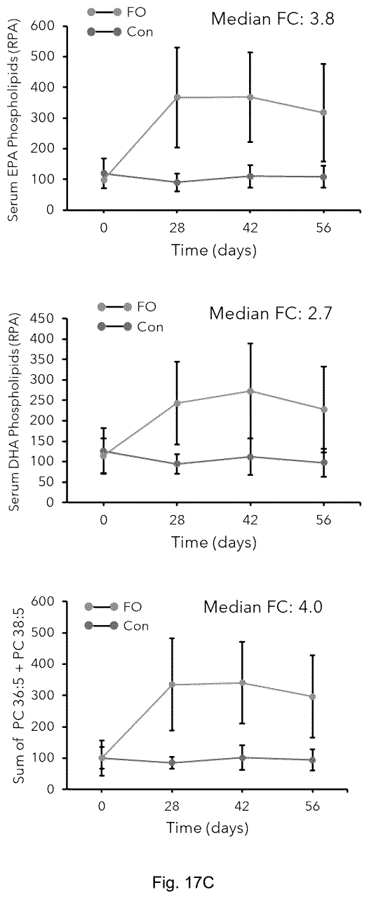

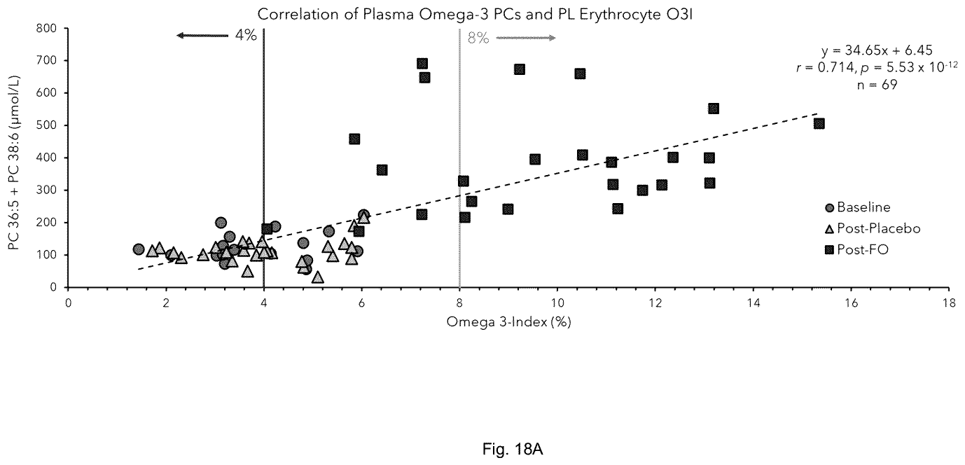

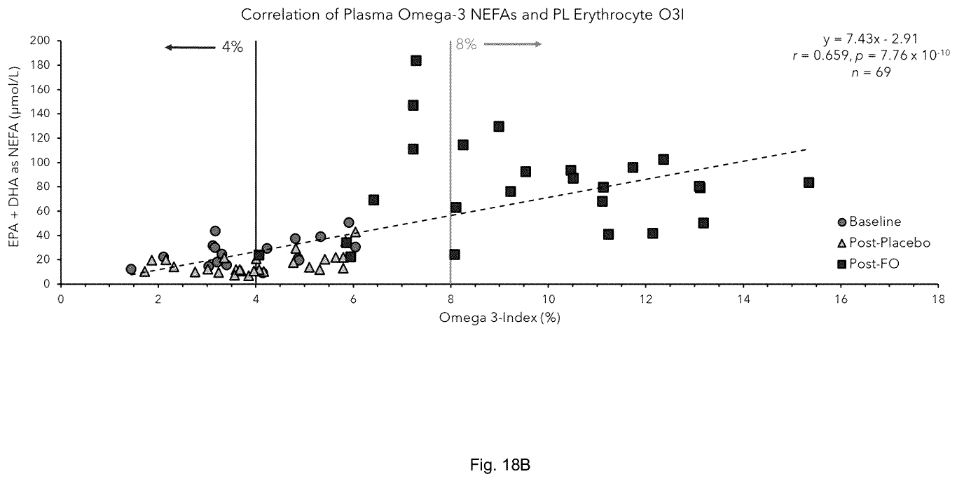

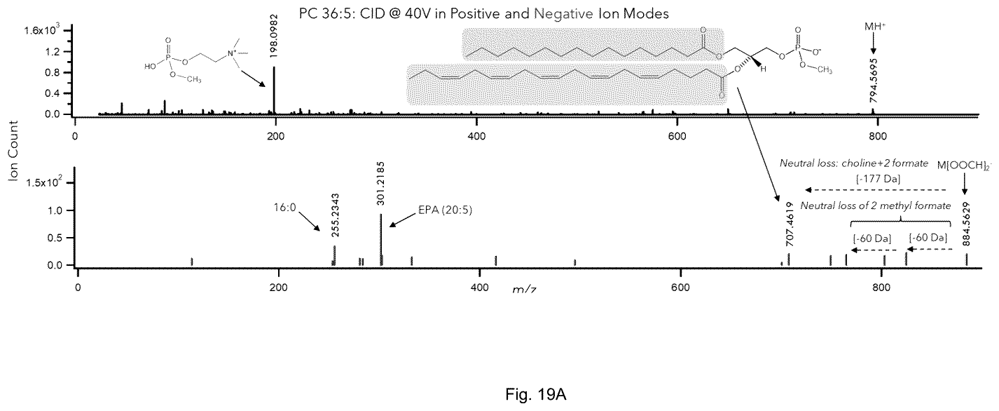

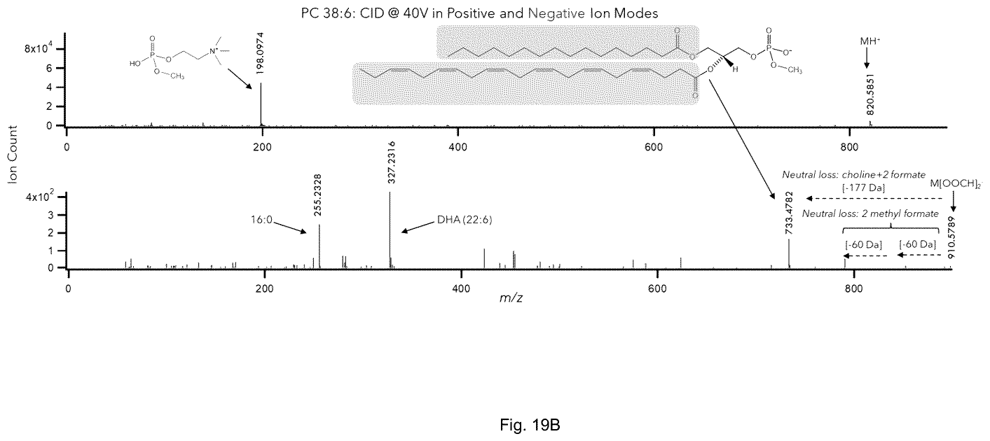

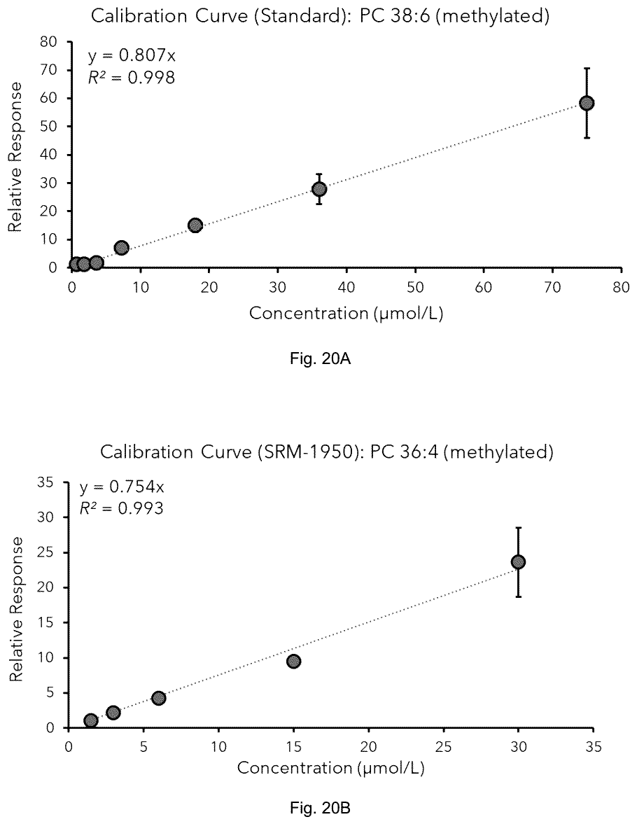

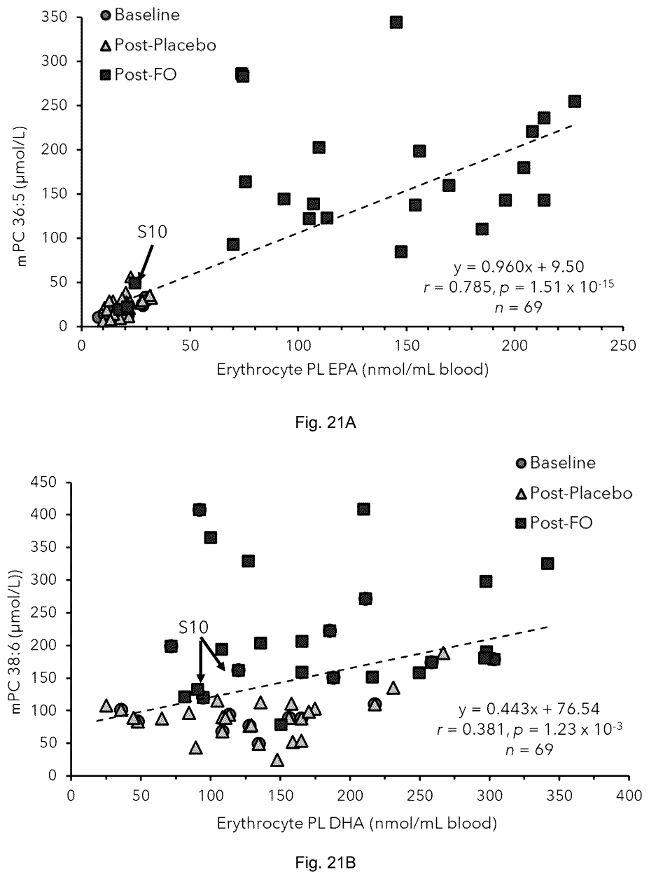

Overall, 44 PC species were quantified consistently from all serum ether extracts in a cohort of young women (n=8) at baseline and then at three time points following high-dose FO or sunflower oil (SO) placebo intervention over 56 days (FIG. 16A). A 2D PCA scores plot with hierarchical cluster analysis (HCA) heat map comprising 44 serum PCs following glog-transformation and autoscaling illustrates the overall data structure (FIG. 16B). Good technical precision was achieved from a repeat analysis of a pooled QC sample (median CV=13%, n=13) as compared to the biological (between-subject) variance in the serum lipidome for all participants (median CV=49%, n=69). FIG. 17 depicts time series trajectories for a series of top-ranked serum biomarkers associated with high-dose FO ingestion as compared to the intake of sunflower oil (SO) as placebo when using a repeat measures mixed 2-way ANOVA model (Table 7). As expected, elevated and steady-state levels of circulating EPA and DHA containing PCs as a single species or their sum were reached within 28 days in the FO treatment arm as compared to placebo. Since SO contains linoleic (FA 18:2) and oleic acid (FA 18:1) as major FA constituents, serum levels of PC 32:1 and PC 36:2 were also included as controls, but they showed no change (p>0.05) in either placebo and FO treatments. Table 7 highlights that a panel of two circulating PC lipid species, namely the sum of PC 36:5 and PC 38:6, generated the greatest effect size (0.851) and statistical significance (p=9.93×10⁻⁷) in response to high-dose FO supplementation relative to placebo unlike other larger PC panels (up to six) or single PC lipid species. Importantly, the sum concentration (umol/L) of serum PC 36:5 and PC 38:6 exhibited a positive correlation (r=0.714, p=5.53×10⁻¹²) to the O3I derived from the wt % of EPA and DHA of PLs in erythrocyte membranes (FIG. 18). In fact, an improved correlation with greater sensitivity to FO intake was achieved for serum PC 36:5+PC 38:6 as compared to EPA+DHA previously measured as their NEFAs (87). These two circulating lipid biomarkers of the O3I were tentatively identified as PC (16:0_20:5) and PC (16:0_22:6) following annotation of their MS/MS spectra in positive and negative ion mode detection (FIG. 19). Serum PC concentrations were also measured with external calibration curves (FIG. 20) using a purified lipid standard (PC 16:0/22:6), or estimated using a surrogate PC (PC 36:4 for PC 36:5) following serial dilution of NIST-SRM 1950 human plasma based on consensus concentrations reported in a lipidomics harmonization study as described in example 1. Temporal changes in lipidome profiles also demonstrated that serum PCs containing EPA fatty acyl chains responded to FO supplementation more than DHA containing PCs. This was reflected by a stronger association for serum PC 36:5 concentrations (umol/L) and EPA erythrocyte PL content (nmol/mL) (r=0.785, p=1.51×10⁻¹⁵) as compared to serum PC 38:6 and DHA erythrocyte PL content (r=0.381, p=1.23×10⁻³) as shown in FIG. 21. The greater sensitivity of serum PC 36:5 following FO intake was also useful to screen for likely dietary non-adherence of a participant (87), who was excluded from subsequent statistical analyses in this study.

### Validation of Serum PC Biomarkers of O3I Status Following DHA or EPA Intake:

As the high-dose FO trial relied on an unequal mixture of n3-LCPUFAs in a modest number of women, it was next aimed to further validate lead candidate PC biomarkers of O3I status in an independent trial involving a larger cohort (n=83) using purified EPA or DHA only supplements at the same dosage level (˜3 g/day over a 56-day period) relative to olive oil (OO) as the placebo. In addition, it was sought to confirm whether the same lipids can be related to the O3I in a different blood fraction, namely human plasma (EDTA as anticoagulant) rather than serum. This cohort comprised young, normal weight, non-smoking Canadian adults of both sexes who had a different (p=5.67×10⁻³) baseline O3I status of (3.77±0.63%) and (3.34±0.76%) for women (n=43) and men (n=40), respectively (Table 8). Also, the mean O3I status at baseline for all participants was (3.50±0.68% ranging from 1.87% to 5.21%) with 75% of participants having an 031<4%. High-dose EPA and DHA intake significantly increased their average O3I status from baseline to (8.30±1.21%) and (6.49±1.17%), respectively as compared to OO placebo (3.61±0.60%) after 90 days. As a result, DHA more effectively increased the O3I than EPA supplementation as reflected by 71% versus 11% of participants achieving a low cardiovascular risk profile of O3I>8%, respectively.

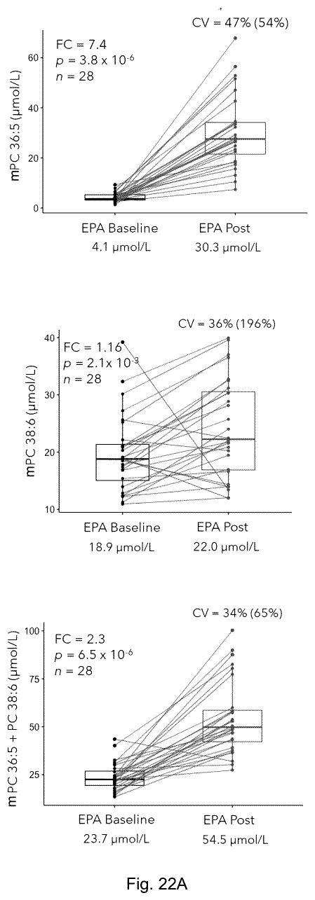

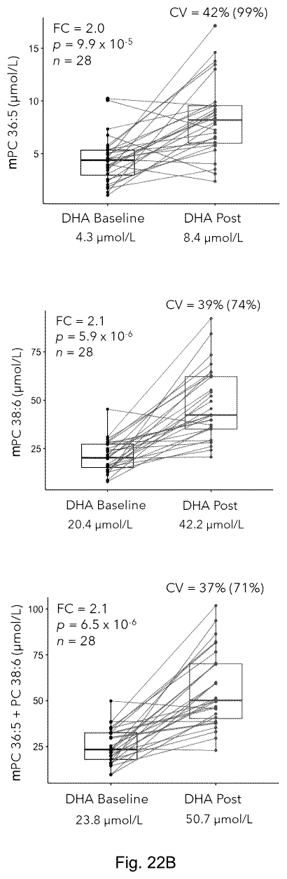

After identifying several n3-LCPUFA containing PC species and panels responsive to FO supplementation in the sub-group screen (Table 6) and full analysis (Table 7), a targeted lipidomic analyses of these same PC biomarker candidates was subsequently performed in a second independent placebo-controlled EPA and DHA only trial. As expected, the same circulating PCs responded to this specific n3-LCPUFA dietary intervention, notably PC 36:5 from a median baseline of 3.4 mmol/L to 23.8 mmol/L (median FC˜7.0) following EPA ingestion as shown in FIG. 22A. The spaghetti box plot also highlights considerable treatment response variability between-subjects, which had a mean CV=48% for PC 36:5 concentrations measured after EPA supplementation alone, or a mean CV=55% based on plasma concentration changes from baseline for individual participants. In contrast, there was a modest increase (˜17%) in DHA-containing PC 38:6 concentrations from baseline after EPA intake from 18.9 mmol/L to 22.0 mmol/L with much larger between-subject treatment response variations (mean CV=182%). This also coincided in a lower treatment response (median FC˜2.2) overall when measuring the sum of plasma PC 36:5 and PC 38:6 concentrations following high-dose EPA supplementation.

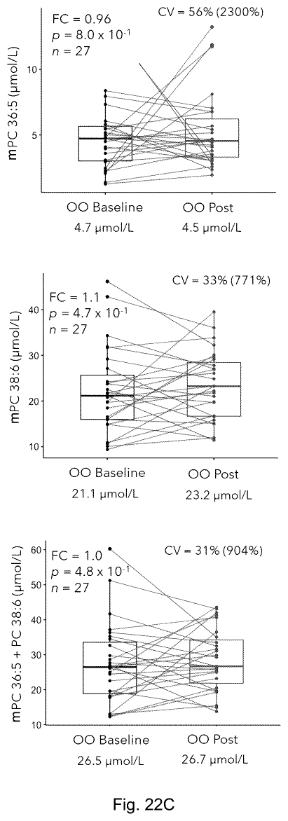

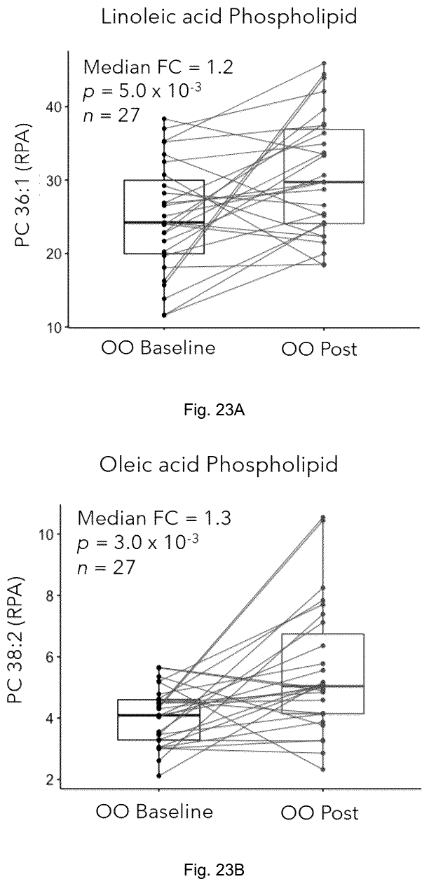

In contrast, ingestion of a high-dose DHA-specific supplement elicited a more attenuated increase in plasma PC 38:6 (median FC˜2.1) from 20.1 mmol/L to 42.3 mmol/L that was similar in magnitude to the EPA-containing PC 36:5 (˜88% increase from baseline) as highlighted in FIG. 22B. This was likely due to the higher (˜4.7-fold) baseline concentrations for plasma PC 38:6 as compared to PC 36:5, thereby being less sensitive to high-dose DHA supplementation. As a result, the sum concentration for plasma PC 36:5 and PC 38:6 generated a similar overall treatment response following DHA intake. As the constituents of OO consisted primarily of linoleic acid and oleic acid, FIG. 22C confirmed no change in either PC 36:5, PC 38:6 or their sum in the placebo arm (p>0.05). However, a modest increase (˜1.2-fold, p˜0.004) in plasma PC 36:1 and PC 38:2 was measured from baseline following OO intake (FIG. 23). Yet, this effect was much lower in magnitude than treatment responses involving the two omega-3 FA containing PCs following high-dose EPA or DHA intake.

### Two Circulating PCs as Surrogate Biomarkers of the O3I:

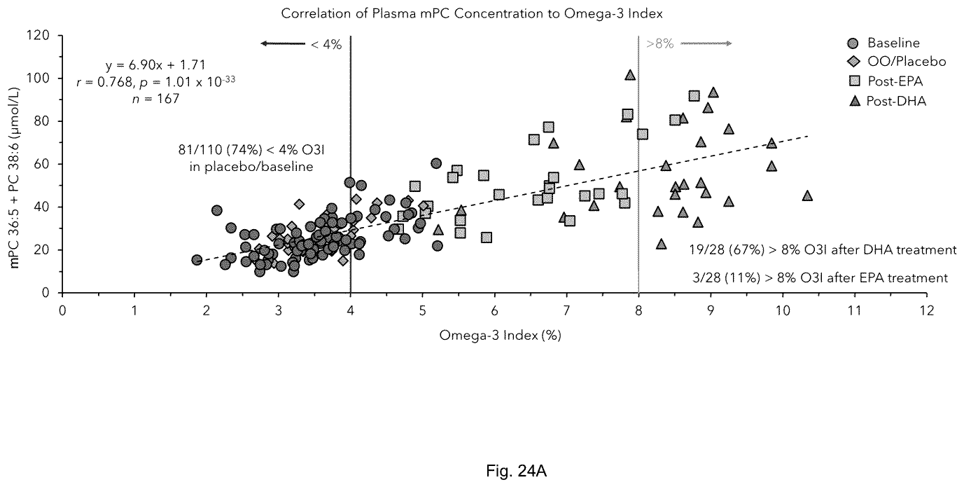

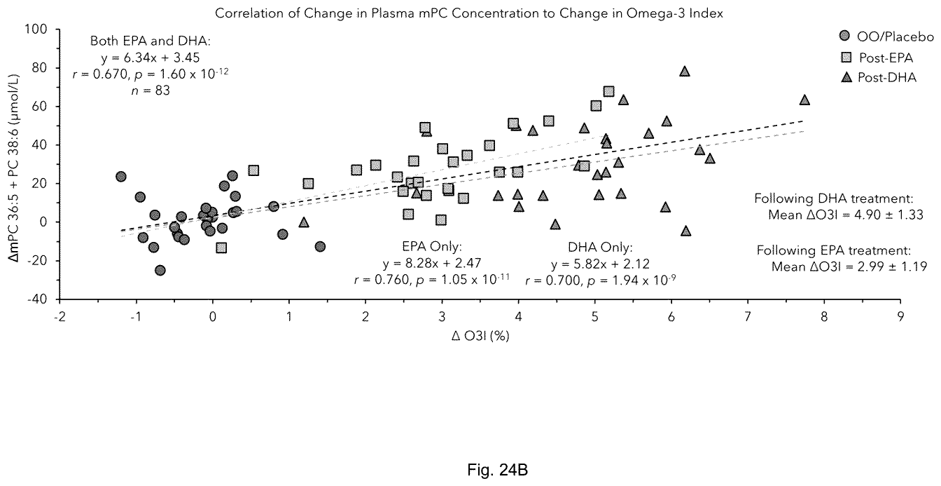

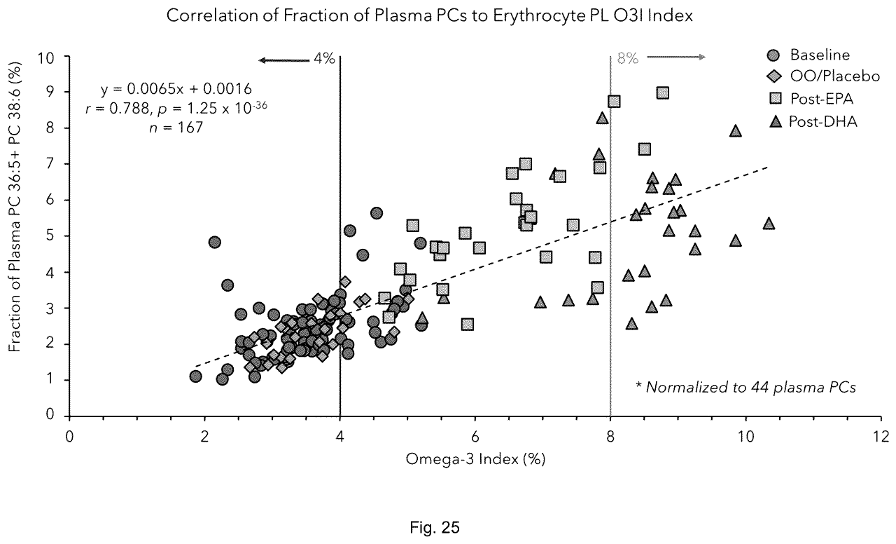

It was next determined whether circulating PCs may serve as potential surrogate measures of erythrocyte PL derived O3I while also reflecting intake of high-dose FO intake or purified supplements of either EPA or DHA. While the total sum of all six n3-LCPUFA containing PC species demonstrated a moderate correlation (r=0.636) to the O3I, statistical outcomes were improved when using fewer PCs within the panel (Table 9). Similar to the outcomes reported from the high-dose FO trial, the strongest correlation to the O3I in this cohort was achieved using the sum concentration for plasma PC 36:5 and PC 38:6, representing two of the most abundant circulating EPA and DHA containing PL species in human blood (89). FIG. 24A depicts a correlation plot for plasma PC 36:5+PC 38:6 based on their absolute concentrations (mmol/L) as a function of O3I (r=0.768, p=1.01 × 10-33), which highlights a distinct enhancement in omega-3 FA nutrition after 90 days of supplementation. Importantly, most participants (˜74% or 81/110 with a mean O3I of 3.56%) had a high-risk O3I profile (<4%) at baseline and after OO supplementation, whereas only 26% were classified as having a moderate risk category (4-8%) with not a single participant having an O3I>8%. In contrast, 67% and 11% of participants following intake of 3.0 g of EPA or DHA had their O3I status changed into a low-risk profile for cardiovascular health (>8% O3I), respectively. Although EPA was less efficacious in increasing the O3I than DHA at the same dosage level, all participants improved to at least a moderate risk category (4-8%). Also, reporting the fraction (%) of PC 36:5+PC 38:6 normalized to a total of 44 plasma PCs measured by MSI-NACE-MS, provided only a modest additional improvement in its association with the O3I (r=0.788, p=1.25x 10-36) as compared to the absolute concentration for two PCs alone (FIG. 25). Additionally, these differential treatment response outcomes were explored by considering EPA and DHA-specific correlations to plasma PC 36:5+PC 38:6 concentration as a function of differences in the O3I status from baseline as depicted in FIG. 24B. As expected, DHA supplementation alone contributed to a 63% greater relative efficacy overall (ΔO3I=4.90±1.33%) as compared to participants ingesting a similar dose of EPA (ΔO3I=2.99+1.19%). This difference in treatment response was also captured by comparing the slopes determined from the correlation of DHA (slope=5.93) and EPA (slope=9.29) only supplement sub-groups using a least-squares linear regression model. This approach may enable correction for the attenuated DHA treatment response relative to EPA within the circulating PC lipid pool as required to estimate their composition within erythrocyte membranes that itself serves as a proxy for cardiac tissue (75,76). Overall, there was a modest sex-dependence (p˜0.02) found in measured changes in plasma PC 36:5+PC 38:6 concentrations and the O3I from baseline (Table 10). Overall, this effect was more pronounced in females ingesting EPA who had greater increases in their circulating concentrations of n3-LCPUFA containing PCs. In contrast, males who ingested DHA had greater changes in the O3I than females reflecting their lower baseline status.

### Discussion:

Epidemiological studies of Greenland Inuit consuming a traditional diet rich in marine organisms first implicated greater n3-LCPUFA intake with a lower incidence of cardiovascular disease than Western dietary patterns (99). However, changes in diet and cultural practices have lowered the omega-3 FA nutritional status of contemporary Inuit coinciding with an epidemiological transition of greater chronic disease burden and psychological distress (100,101). Several prospective studies in other populations have reported that low fish/seafood consumption and poor n3-LCPUFA nutrition is associated with higher all-cause and cardiovascular mortality (102-105), with EPA demonstrating the strongest association independent of other risk factors (106). Indeed, clinical trials involving purified high-dose (˜3-4 g/day) EPA and its analogs provide growing evidence of its utility as an adjunct therapy for the prevention of major coronary events in high-risk patients (107,108) by reducing circulating triglyceride levels, as well as vascular inflammation as compared to DHA alone or DHA+EPA mixtures (109). Thus, EPA and DHA have overlapping and divergent effects on gene expression (110), membrane structure (111), lipogenesis (85), and cellular metabolism in subjects with chronic inflammation (112). As a result, objective biomarkers of n3-LCPUFA intake are urgently needed to measure these conditionally essential FAs during the lifespan as they are not reliably quantified by questionnaires given the variability in their amount, quality and composition in dietary fats (113).

To date, a major challenge in using the O3I as a risk assessment tool in clinical medicine is the variety of analytical methods (e.g., specimen type, extraction procedure, fractionation etc.) used for measuring n3-LCPUFAs from different circulating lipid pools, including erythrocytes, plasma total lipids, plasma PL fraction, and whole blood (114,115). Although the gold standard for O3I determination remains GC analysis of FAMEs from the PL fraction of erythrocytes isolated after thin-layer chromatography, this procedure is both time consuming and less amenable to high throughput screening (75-77). Also, the total number of reported fatty acids (up to 50) can vary widely between methods, which complicates standardization and data comparisons when reporting the sum of EPA and DHA as their wt % (103). Alternatively, 1H-NMR may enable the reliable estimation of O3I status in large-scale prospective studies based on the analysis of DHA % and non-DHA % plasma lipoproteins with a good mutual agreement to GC results (116). However, neither GC or NMR methods directly resolve and quantify specific intact lipid species in small volumes of blood specimens that are best achieved when using chromatographic, ion mobility or electrophoretic separations coupled to high resolution MS (117). Herein, a high throughput lipidomic platform based on MSI-NACE-MS was applied under two configurations that takes advantage of serial injection of seven serum/plasma extracts in a single analytical run as shown in example 1 (88,89). MSI-NACE-MS allows for unique data workflows by encoding mass spectral information temporally within a separation when performing untargeted lipidomics. For instance, this approach was used to reliably authenticate and identify lipid features that increased following high-dose FO intake in a pooled sub-group analysis, which was subsequently validated in two randomized placebo-controlled trials, including EPA or DHA only supplementation. Although a two-stage FMOC/MTT derivatization procedure is required to generate cationic methylated PCs from serum/plasma ether extracts prior to MSI-NACE-MS analyses, this is far less hazardous than using diazomethane previously reported to improve the chromatographic performance, as well as enhance the selectivity and sensitivity for glycerophospholipid and sphingolipid analyses by LC-MS/MS (98,118). In general, MSI-NACE-MS offers better selectivity than HILIC-MS methods since polar/ionic lipids are resolved based on differences not only in their polar head group, but also bond linkage and total acyl chain length that impact their apparent electrophoretic mobility as described in example 1. However, type-II isobaric interferences may occur if not verified or corrected for in complex biological samples due to co-migration of PLs having differences in the number of double bonds (FIG. 15), which can be minimized with higher resolution mass analyzers and optimal data pre-processing (119).

Recently, Dawzynski et al. (120) reported that dietary polyunsaturated fatty acids predominately increased several DHA-containing plasma phosphatidylethanolamines (PEs) and plasmalogens following consumption of algal oil as a vegetarian marine source of n3-LCPUFAs in a small number of participants. In contrast, it was found that most circulating classes of ionic lipids measured by MSI-NACE-MS, including omega-3 FA containing PEs (e.g., PE 38:5, PE 38:6), LPEs (LPE 20:5, LPE 22:6), PIs (e.g., PI 40:6, PI 40:7) and LPCs (e.g., LPC 20:5, LPC 22:6) did not exhibit increases following high-dose FO supplementation with the exception of EPA and DHA as their NEFAs, but not DPA (FIGS. 13, 14, Table 6). These discordant results may be due to differences in marine supplement/composition (1.6 g/day DHA intake with unreported EPA content) and assay selectivity, as plasma lipidome changes were analyzed by direct infusion-MS/MS without chromatographic separation thereby being more prone to isobaric/isomeric interferences (120). Additionally, other phytochemicals and fat-soluble vitamin constituents present in algal oil may elicit distinct plasma lipidome changes in humans as compared to FO sources or purified DHA or EPA only supplements, including their predominate lipid form that impacts bioavailability (e.g., triglyceride versus phospholipid). Nevertheless, the findings were replicated in two independent trials that demonstrated that the sum concentration of PC 36:5 and PC 38:6 was most significantly correlated to the O3I as compared to other PC panels or a single PC species alone (Table 6, Table 9) when using a validated MSI-NACE-MS platform and a robust data workflow for credentialing ionic lipids (88). In this case, zwitter-ionic PCs were preferentially measured as their methylated cationic lipid derivatives with improved separation resolution and ionization response in MSI-NACE-MS under positive ion mode as described in example 1. However, similar outcomes were measured for the same pair of underivatized PCs analyzed directly under negative ion mode (Table 6). Overall, lipidomic studies by MSI-NACE-MS demonstrated acceptable technical precision with a median CV=13% as compared to the larger biological variance based on 44 circulating PCs consistently measured in most participants (FIG. 16). The two PC species identified as surrogate biomarkers of the O3I, namely PC 16:0_22:5 and PC 16:0_22:6, were characterized with high confidence by MS/MS after collision-induced dissociation experiments under positive and negative ion mode detection (FIG. 19). However, not all lipid species associated with FO, EPA or DHA intake in this study comprised single resolved PC molecular species in MSI-NACE-MS, such as PC 38:5 that is comprised of two co-migrating ions previously shown to be composed of PC 16:0_22:5 and PC 18:1_20:4 as shown in example 1. This confounding effect may explain the poorer performance for certain PCs as putative O3I biomarkers (Table 7) when compared to fully resolved species in MSI-NACE-MS that lack isobaric/isomeric interferences. Nevertheless, independent replication using an orthogonal reversed-phase LC-MS/MS lipidomics method is warranted to further validate the findings in this study.

Among young, normal weight and otherwise healthy Canadian adults recruited in the placebo-controlled EPA and DHA-specific supplementation trial, their average O3I at baseline/placebo was (3.50±0.68%) with most participants (74%) classified as having an 031<4% (FIG. 24A). However, females had higher baseline 031 than males likely due to estrogenic effects that have been reported to upregulate DHA biosynthesis in women especially when taking oral contraceptives (121). In fact, most childbearing age and pregnant women do not meet their recommended dietary intake of omega-3 FAs, (122) which can increase the risk for premature and low-weight births (123). The O3I status in this cohort is considerably lower than a previous 2012-2013 household survey of Canadian adults (20 to 79 years) reporting an average O3I of 4.5% with only 42% having <4% O3I (22), similar to data from a UK biobank study (116), and a dietary intervention involving company employees in Germany (124). All three studies reported a higher O3I status in women and older persons, including participants who regularly consumed fish and/or omega-3 FA supplements, but were not obese, and did not smoke tobacco. In fact, Stark et al. (121) reported that a suboptimal O3I status (<4% O3I) is prevalent in most global populations except for high consumers of seafood in Japan, Korea, Scandinavia and certain indigenous groups not fully adapted to Western foods. Nevertheless, only modest increases in O3I have been achieved by increasing the intake of omega-3 FA rich seafood even in participants motivated to monitor their 031 status (124), with at least 3 fish servings per week plus dietary supplement use needed to achieve an O3I>8% that exceeds current guidelines by the American Heart Association (125). This work confirmed that high-dose FO (3 g/day EPA+2 g/day DHA), and EPA or DHA-only supplements (3 g/day) significantly improved the O3I status in most study participants. However, there were considerable variations in treatment responses measured for circulating concentrations of PC 36:5 and PC 38:6 with a CV ranging from 55 to 73% for EPA and DHA only supplementation, respectively (FIG. 22). Overall, FO and DHA only supplements were most effective to increase O3I>8% (˜64-72%) in young Canadian adults as compared to EPA alone (˜11%) with DHA having a slightly greater impact in men than women reflecting their lower baseline O3I status (Table 10). These results are consistent with the greater potency and sex-dependence reported for DHA supplementation as compared to EPA (126). However, it is unclear how specific increases in O3I that reflect changes in lipid membrane composition of erythrocytes are related to modulating long-term cardiovascular risk given the distinct mechanisms of action of EPA and DHA in the body.

Overall, it was demonstrated that serum concentrations for PC 36:5 and PC 38:6 had a better correlation with greater sensitivity to detect changes in O3I than the sum of DHA and EPA as their NEFAs (FIG. 18). Moreover, measured plasma concentrations for just these two circulating PCs retained most of their association with O3I, and only a marginal improvement was gained when reporting their fraction normalized to 44 PCs (FIG. 25) which greatly simplifies and standardizes reporting. The differences in EPA and DHA efficacy for augmenting O3I status reflect the 50% higher dosage of EPA in FO as compared to DHA, as well as the much lower content of EPA within erythrocyte membranes at baseline prior to supplementation (FIG. 21). This indicates that serum or plasma PC 36:5 may serve as a more sensitive blood biomarker for monitoring adherence to dietary/supplemental FO intake, as well as an increasing number of EPA specific therapeutic applications (51-54). Indeed, recent studies have confirmed that baseline 031 status, dose and exact lipid formulation are primary factors that explain about 62% of the total variance in treatment responses to omega-3 FA supplements (127). In this study, treatment response variations were attributed mainly to biological sex, as well as genotype differences that have been reported to effect fatty acyl desaturase and elongase activity, apolipoprotein E transport and eicosanoid production (128). As expected, there were no changes in PC 36:5, PC 38:6 or their sum following intake of OO as placebo (FIG. 22C), however modest increases were measured in linoleic acid containing PC 36:1 and an oleic acid containing PC 38:2 from baseline (FIG. 23). Overall, DHA supplementation elicited a 64% greater increase in the O3I from baseline as compared to EPA at the same dose level (FIG. 24A). Moreover, estimation of O3I status (126) from plasma PC 36:5 and PC 38:6 concentrations may be achieved by use of EPA and DHA specific calibration curves (FIG. 24B). Recent studies highlight the distinctive effects that EPA containing PCs have on membrane fluidity and structure than DHA alone, DHA/EPA mixtures, or omega-6 containing PCs, such as arachidonic acid (AA) (111). Indeed, Iwamatsu et al. (129) reported that the serum ratio of EPA to AA, but not DHA to AA, provided improved predictive accuracy as risk biomarkers of coronary artery disease, especially in patients with acute coronary syndrome.

### Conclusion:

In summary, the impact of high-dose n3-LCPUFA supplementation using FO, EPA and DHA specific formulations, was explored on global changes in the blood lipidome profiles of healthy young adults. An accelerated data workflow was first applied when using MSI-NACE-MS to identify putative circulating lipid biomarkers associated with high-dose FO intake in a pooled sub-group analysis subsequently validated in two independent placebo-controlled trials. The sum of only two circulating PCs, namely PC 16:0_20:5 and PC 16:0_22:6 in serum or plasma, provided the strongest correlation to the O3I that reflects local changes in erythrocyte membrane composition and cellular function after a minimum of 28 days of supplementation. However, PC 16:0_20:5 was more sensitive to omega-3 FA supplementation than PC 16:0_22:6 despite DHA intake generating greater changes in the O3I from baseline. Although MSI-NACE-MS was used for the discovery of circulating biomarkers of the O3I, other lipidomic platforms can also be used for their routine screening in small volumes of blood, including ion mobility-MS/MS and LC-MS/MS. The potential for non-invasive assessment of the O3I and its physiological effects following EPA and/or DHA supplementation in urine specimens may allow for more convenient population screening. Future work will further validate these findings in a larger prospective cohort since circulating lipid pools of EPA and DHA are modifiable dietary risk factors correlated with longevity and vascular health. This work is critical to guide evidence-based dietary and lifestyle interventions for optimal health outcomes on an individual level.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

TABLE 1
Annotated plasma phospholipids (n = 75) measured from NIST-SRM
1950 by MSI-NACE-MS that satisfied acceptance criteria reported in
the Bowden et al. 2017 lipidomics harmonization study. PC species
were reported in the harmonization study as a combined isomer panel
with plasmanyl (PC-O) and plasmenyl (PC-P) phospholipids are noted
with an asterisk (*), but they were confirmed as not detected in this study.

| | | | | Electrophoretic | |
|---|---|---|---|---|---|
| | | | Mass | Mobility | Molecular |
| Lipid | Derivatized | Actual | Error | (cm²/Vs) × | Formula |
| Species | m/z | Mass | (ppm) | 10⁻⁴ | [Methylated] |
| PC 30:0 | 720.5538 | 720.5518 | −2.71 | 2.142 | C39H79NO8P |
| PC 31:2* | 730.5382 | 730.5376 | −0.75 | 2.140 | C40H77NO8P |
| PC 31:1* | 732.5538 | 732.5512 | −3.48 | 2.128 | C40H79NO8P |
| PC 31:0* | 734.5695 | 734.5624 | −9.60 | 2.100 | C40H81NO8P |
| PC 32:3 | 742.5382 | 742.5394 | 1.68 | 2.137 | C41H77NO8P |
| PC 32:2* | 744.5538 | 744.5478 | −7.99 | 2.120 | C41H79NO8P |
| PC 32:1 | 746.5695 | 746.5653 | −5.56 | 2.106 | C41H81NO8P |
| PC 32:0 | 748.5851 | 748.5798 | −7.01 | 2.085 | C41H83NO8P |
| PC 33:3* | 756.5538 | 756.5608 | 9.32 | 2.100 | C42H79NO8P |
| PC 33:2* | 758.5695 | 758.5742 | 6.26 | 2.066 | C42H81NO8P |
| PC 33:1* | 760.5851 | 760.5859 | 1.12 | 2.050 | C42H83NO8P |
| PC 33:0* | 762.6008 | 762.5958 | −6.49 | 2.051 | C42H85NO8P |
| PC 34:5* | 766.5382 | 766.5341 | −5.28 | 2.121 | C43H77NO8P |
| PC 34:4* | 768.5538 | 768.55 | −4.88 | 2.095 | C43H79NO8P |
| PC 34:3* | 770.5695 | 770.5668 | −3.44 | 2.066 | C43H81NO8P |
| PC 34:2* | 772.5851 | 772.5835 | −2.01 | 2.049 | C43H83NO8P |
| PC 34:1 | 774.6008 | 774.6041 | 4.32 | 2.036 | C43H85NO8P |
| PC 34:0 | 776.6164 | 776.6084 | −10.24 | 2.038 | C43H87NO8P |
| PC 35:6* | 778.5382 | 778.5457 | 9.70 | 2.040 | C44H77NO8P |
| PC 35:5* | 780.5902 | 780.6045 | 18.38 | 2.092 | C44H79NO8P |
| PC 35:4* | 782.6059 | 782.5923 | −7.31 | 2.057 | C44H81NO8P |
| PC 35:3* | 784.6215 | 784.6029 | −3.64 | 2.031 | C44H83NO8P |
| PC 35:2* | 786.6372 | 786.6213 | −8.15 | 2.019 | C44H85NO8P |
| PC 35:1* | 788.6528 | 788.6235 | −7.09 | 2.006 | C44H87NO8P |
| PC 36:6 | 792.5538 | 792.5529 | −1.07 | 2.049 | C45H79NO8P |
| PC 36:5 | 794.5695 | 794.5594 | −12.65 | 2.042 | C45H81NO8P |
| PC 36:4 | 796.5851 | 796.5834 | −2.07 | 2.037 | C45H83NO8P |
| PC 36:3 | 798.6008 | 798.6018 | 1.31 | 2.014 | C45H85NO8P |
| PC 36:2 | 800.6164 | 800.6123 | −5.06 | 1.988 | C45H87NO8P |
| PC 36:1 | 802.6321 | 802.6372 | 6.42 | 1.979 | C45H89NO8P |
| PC 36:0* | 804.6477 | 804.6363 | −4.11 | 1.975 | C45H91NO8P |
| PC 37:6* | 806.5695 | 806.5703 | 1.05 | 2.030 | C46H81NO8P |
| PC 37:5* | 808.5851 | 808.5925 | 9.21 | 2.015 | C46H83NO8P |
| PC 37:4* | 810.6008 | 810.6009 | 0.19 | 1.986 | C46H85NO8P |
| PC 37:3* | 812.6164 | 812.6074 | −11.01 | 1.979 | C46H87NO8P |
| PC 37:2* | 814.6321 | 814.6281 | −4.85 | 1.968 | C46H89NO8P |
| PC 38:6 | 820.5851 | 820.5828 | −2.74 | 1.996 | C47H83NO8P |
| PC 38:5 | 822.6008 | 822.596 | −5.77 | 1.985 | C47H85NO8P |
| PC 38:4 | 824.6164 | 824.6153 | −1.27 | 1.975 | C47H87NO8P |
| PC 38:3 | 826.6321 | 826.6256 | −7.80 | 1.962 | C47H89NO8P |
| PC 38:2 | 828.6477 | 828.6443 | −4.04 | 1.939 | C47H91NO8P |
| PC 39:7* | 832.5851 | 832.5971 | 4.47 | 1.984 | C48H83NO8P |
| PC 39:5* | 836.6164 | 836.6099 | −7.71 | 1.948 | C48H87NO8P |
| PC 40:8 | 844.5851 | 844.5857 | 0.77 | 1.999 | C49H83NO8P |
| PC 40:7 | 846.6008 | 846.5933 | −8.80 | 1.954 | C49H85NO8P |
| PC 40:6 | 848.6164 | 848.6146 | −2.06 | 1.933 | C49H87NO8P |
| PC 40:5 | 850.6321 | 850.633 | 1.12 | 1.920 | C49H89NO8P |
| PC 40:4 | 852.6477 | 852.6474 | −0.29 | 1.917 | C49H91NO8P |
| SM 32:2; O2 | 687.5436 | 687.5316 | −7.38 | 1.979 | C38H76N2O6P |
| SM 32:1; O2 | 689.5592 | 689.5532 | −8.63 | 1.976 | C38H78N2O6P |
| SM 33:1; O2 | 703.5749 | 703.5685 | −9.03 | 1.956 | C39H80N2O6P |
| SM 34:2; O2 | 715.5749 | 715.5746 | −0.35 | 1.923 | C40H80N2O6P |
| SM 34:1; O2 | 717.5905 | 717.5886 | −2.58 | 1.916 | C40H82N2O6P |
| SM 34:0; O2 | 719.6062 | 719.5999 | −8.69 | 1.916 | C40H84N2O6P |
| SM 36:3; O2 | 741.5905 | 741.5911 | 0.88 | 1.884 | C42H82N2O6P |
| SM 36:2; O2 | 743.6062 | 743.6078 | 2.22 | 1.872 | C42H84N2O6P |
| SM 36:1; O2 | 745.6218 | 745.6162 | −7.44 | 1.866 | C42H86N2O6P |
| SM 36:0; O2 | 747.6375 | 747.6334 | −5.42 | 1.813 | C42H88N2O6P |
| SM 37:1; O2 | 759.6375 | 759.6326 | −6.38 | 1.854 | C43H88N2O6P |
| SM 38:3; O2 | 769.6218 | 769.6324 | 3.84 | 1.820 | C44H86N2O6P |
| SM 38:2; O2 | 771.6375 | 771.6292 | −10.69 | 1.801 | C44H88N2O6P |
| SM 38:1; O2 | 773.6531 | 773.6426 | −13.51 | 1.768 | C44H90N2O6P |
| SM 39:2; O2 | 785.6531 | 785.6445 | −10.88 | 1.783 | C45H90N2O6P |
| SM 39:1; O2 | 787.6688 | 787.6634 | −6.79 | 1.768 | C45H92N2O6P |
| SM 40:3; O2 | 797.6531 | 797.6523 | −0.94 | 1.755 | C46H90N2O6P |
| SM 40:2; O2 | 799.6688 | 799.6628 | −7.44 | 1.747 | C46H92N2O6P |
| SM 40:1; O2 | 801.6844 | 801.6764 | −9.92 | 1.722 | C46H94N2O6P |
| SM 41:3; O2 | 811.6688 | 811.6622 | −8.07 | 1.750 | C47H92N2O6P |
| SM 41:2; O2 | 813.6844 | 813.6793 | −6.21 | 1.744 | C47H94N2O6P |
| SM 41:1; O2 | 815.7001 | 815.6894 | −13.06 | 1.734 | C47H96N2O6P |
| SM 42:3; O2 | 825.6844 | 825.6804 | −4.78 | 1.724 | C48H94N2O6P |
| SM 42:2; O2 | 827.7001 | 827.6957 | −5.26 | 1.730 | C48H96N2O6P |
| SM 42:1; O2 | 829.7157 | 829.7111 | −5.48 | 1.713 | C48H98N2O6P |
| SM 43:2; O2 | 841.7157 | 841.7108 | −5.76 | 1.728 | C49H98N2O6P |
| SM 44:2; O2 | 855.7314 | 855.7512 | 3.20 | 1.701 | C50H100N2O6P |

¹ Bowden et al. J. Lipid Res. 2017 58: 2275-2288.

TABLE 2
Plasma phospholipids from NIST SRM-1950 measured by MSI-NACE-MS that
did not satisfy acceptance criteria in the Bowden et al. 2017 lipidomics
harmonization study. All plasma phospholipid masses and mobility measurements
are based on their cationic methylated phosphoesters.

| | | | | Electrophoretic | |
|---|---|---|---|---|---|
| | | | Mass | Mobility | Molecular |
| Lipid | Derivatized | Actual | Error | (cm²/Vs) × | Formula |
| Species | m/z | Mass | (ppm) | 10⁻⁴ | [Methylated] |
| PC 30:1 | 718.5382 | 718.5376 | −0.77 | 2.144 | C39H77NO8P |
| PC 38:1 | 830.6634 | 830.6596 | −4.51 | 1.930 | C47H93NO8P |
| PC 39:6 | 834.6008 | 834.6042 | 4.13 | 1.925 | C48H85NO8P |
| SM 38:0; O2 | 775.6688 | 775.6788 | 12.96 | 1.764 | C44H92N2O6P |
| SM 40:0; O2 | 803.7001 | 803.7122 | 15.12 | 1.717 | C46H96N2O6P |
| SM 42:4; O2 | 827.7001 | 827.6912 | −10.69 | 1.730 | C48H96N2O6P |
| SM 44:3; O2 | 853.7157 | 853.7038 | −13.88 | 1.709 | C50H98N2O6P |

TABLE 3
MSI-NACE-MS validation experiments for select plasma PCs from NIST SRM-1950 compared to consensus
concentrations from various untargeted LC-MS lipidomic methods in different labs in Bowden
et al. (2017) and a targeted shotgun-MS lipidomic assay by Thompson et al. (2020).

| | | | % Bias (n = 5) | | | | |
|---|---|---|---|---|---|---|---|
| | | | to plasma PL | | | | |
| | | | conc. from | | MSI-NACE-MS | | Bowden et al. (2017) |
| | | | Bowden et al. | | | Serial | Harmonization |
| | | | | Serial | | Dilution | Study |
| | | | | Dilution | External | of SRM- | Consensus |
| | Derivatized | Spike & | External | of SRM- | Calibration | 1950 | Concentration |
| Lipid | m/z | Recovery | Calibration | 1950 | (μM) | (μM) | (μM) |
| PC 30:0 | 720.5538 | SRM-1950 | 49.9% | −82.8% | 2.6 ± 0.7 | 3.1 ± 0.8 | 1.6 ± 0.6 |
| | | High Spike | 7.2% | 30.7% | | | |
| | | Mid Spike | 10.2% | 34.4% | | | |
| | | Low Spike | 18.3% | 44.2% | | | |
| | | Average | 11.9% | 49.3% | 2.2 ± 0.6 | 1.3 ± 0.4 | 2.1 ± 0.8 |
| PC 34:0 | 776.6164 | SRM-1950 | 2.1% | −38.4% | | | |
| | | High Spike | −5.0% | −42.7% | | | |
| | | Mid Spike | −1.6% | −40.7% | | | |
| | | Low Spike | −0.6% | −40.1% | | | |
| | | Average | −1.3% | 40.6% | 41.2 ± 3.9 | 41.9 ± 3.3 | 41.0 ± 8.6 |
| PC 38:6 | 820.5851 | SRM-1950 | 0.5% | 2.3% | | | |
| | | High Spike | 8.3% | −6.7% | | | |
| | | Mid Spike | 4.7% | 6.5% | | | |
| | | Low Spike | 24.1% | 26.3% | | | |
| | | Average | 9.4% | 7.1% | 13.2 ± 2.1 | 13.2 ± 2.1 | 14.0 ± 5.1 |
| PC 40:6 | 848.6164 | SRM-1950 | −5.6% | −6.1% | | | |
| | | High Spike | −13.2% | −13.7% | | | |
| | | Mid Spike | −5.3% | −5.9% | | | |
| | | Low Spike | 4.9% | 4.3% | | | |
| | | Average | −8.0% | 0.6% | | | |

| | | | | | | Relative Response Factor | |
|---|---|---|---|---|---|---|---|
| | | | | | | | Serial |
| | | | Thompson et al. (2019) | | | | dilution |
| | | | p400 - | | | External | of NIST |
| | Bowden et al. (2017) | | Reported | p400 | | Calibration, | 1950, |
| | #Labs | COD | Concentration | Bias | LOD | Linearity | Linearity |
| Lipid | Detected | (%) | (μM) | (%) | (μM) | (slope; R²) | (slope; R²) |
| PC 30:0 | 11 | 20 | 1.8 ± 0.6 | 39.3% | 0.70 | 0.754 μM⁻¹, | 0.618 μM⁻¹, |
| | | | | | | 0.999 | 0.990 |
| PC 34:0 | 12 | 18 | NA | NA | 0.08 | 0.867 μM⁻¹, | 1.438 μM⁻¹, |
| | | | | | | 0.998 | 0.979 |
| PC 38:6 | 18 | 11 | 33.5 ± 8.6 | 23.1% | 0.08 | 0.821 μM⁻¹, | 0.807 μM⁻¹, |
| | | | | | | 0.999 | 0.997 |
| PC 40:6 | 17 | 19 | 14.9 ± 5.1 | −11.2% | 0.07 | 0.698 μM⁻¹, | 0.702 μM⁻¹, |
| | | | | | | 0.999 | 0.978 |

TABLE 4
Plasma phospholipids (PCs, n = 14; SMs, n = 7) from NIST
SRM-1950 measured by MSI-NACE-MS following a serial dilution to
estimate their relative response factor using consensus concentrations.¹⁵

| | | | | | # | |
|---|---|---|---|---|---|---|
| | | Consensus | | Response | Calibrant | |
| Lipid | Methylated | Concentration² | # Labs | Factor³ | Data | Linearity |
| Species¹ | m/z | (mM) | Detected | (mM⁻¹) | Points³ | (R²) |
| PC 30:0 | 720.5538 | 1.6 | 11 | 0.618 | 3 | 0.999 |
| PC 34:1 | 774.6008 | 120 | 19 | 1.451 | 6 | 0.989 |
| PC 34:0 | 776.6164 | 2.1 | 12 | 1.438 | 4 | 0.979 |
| PC 36:4 | 796.5850 | 150 | 19 | 0.862 | 6 | 0.993 |
| PC 36:3 | 798.6008 | 100 | 17 | 1.176 | 6 | 0.992 |
| PC 36:2 | 800.6164 | 140 | 18 | 1.495 | 6 | 0.990 |
| PC 36:1 | 802.6320 | 26 | 17 | 2.17 | 5 | 0.990 |
| PC 38:6 | 820.5850 | 41 | 18 | 0.821 | 5 | 0.999 |
| PC 38:5 | 822.6008 | 42 | 18 | 0.896 | 5 | 0.990 |
| PC 38:4 | 824.6164 | 84 | 18 | 1.093 | 5 | 0.990 |
| PC 38:3 | 826.6320 | 26 | 14 | 2.01 | 5 | 0.981 |
| PC 40:6 | 848.6164 | 14 | 17 | 0.702 | 4 | 0.978 |
| PC 40:5 | 850.6320 | 6.7 | 18 | 1.695 | 5 | 0.989 |
| PC 40:4 | 852.6476 | 2.9 | 18 | 1.961 | 5 | 0.993 |
| SM 34:1; O2 | 717.5904 | 100 | 21 | 0.241 | 5 | 0.984 |
| SM 36:1; O2 | 745.6218 | 20 | 22 | 0.320 | 4 | 0.992 |
| SM 40:2; O2 | 799.6688 | 12 | 15 | 0.714 | 4 | 0.975 |
| SM 40:1; O2 | 801.6844 | 20 | 17 | 0.74 | 4 | 0.983 |
| SM 42:3; O2 | 825.6844 | 17 | 12 | 0.625 | 4 | 0.971 |
| SM 42:2; O2 | 827.7000 | 44 | 18 | 0.579 | 5 | 0.970 |
| SM 42:1; O2 | 829.7156 | 20 | 21 | 0.720 | 4 | 0.982 |

¹Annotated lipid species/isobars from NIST SRM-1950 consistently measured by various LC-MS methods in an inter-laboratory lipidomics harmonization study by Bowden et al.¹⁵
²Reported consensus plasma phospholipid concentrations determined by a median of the means from at least 5 different labs having an overall COV < 40%.
³Relative response factors for each plasma phospholipid species following a serial dilution of NIST SRM-1950 to derive a linear calibration curve by MSI-NACE-MS with a minimum of 4 concentration levels (except for PC 30:0).

TABLE 5
Inter-laboratory method comparison of consensus plasma lipids (n = 46) reported by Bowden et
al. (2017) and their concentrations estimated by serial dilution of NIST SRM-1950 when using MSI-
NACE-MS under positive ion mode after methylation. In most cases, a response factor for the closest
matching plasma lipid was used that had a minimum of 4 calibrant points detected upon serial dilution.
Note that an asterisk (*) is used to denote lipid species whose concentrations were estimated using
response factors from a closest surrogate lipid via a serial dilution of NIST-SRM 1950.

| | Consensus | | Lipid Used for | MSI-NACE-MS | | | |
|---|---|---|---|---|---|---|---|
| | Concentration | Derivatized | Response | Concentration | CV (%) | COD | Bias |
| Lipid Species¹ | (μM)² | m/z | Factor | (μM)³ | n = 3 | (%) | (%)⁴ |
| PC 36:4 | 150 ± 28 | 796.5851 | PC 36:4 | 128 ± 26 | 20 | 20 | −15 |
| PC 36:2 | 140 ± 25 | 800.6164 | PC 36:2 | 111 ± 25 | 23 | 15 | −21 |
| PC 34:1 | 120 ± 21 | 774.6008 | PC 34:1 | 101 ± 21 | 21 | 14 | −16 |
| SM 34:1; O2 | 100 ± 15 | 717.5905 | SM 34:1; O2 | 99.6 ± 1.0 | 1.0 | 18 | −0.40 |
| PC 36:3 | 100 ± 14 | 798.6008 | PC 36:3 | 84 ± 18 | 21 | 17 | −16 |
| PC 38:4 | 84 ± 14 | 824.6164 | PC 38:4 | 68 ± 15 | 22 | 17 | −19 |
| SM 42:2; O2 | 44 ± 11 | 827.7001 | SM 42:2; O2 | 39.1 ± 2.8 | 7.1 | 19 | −11 |
| PC 38:5 | 42 ± 7.9 | 822.6008 | PC 38:5 | 34.5 ± 6.3 | 18 | 14 | −18 |
| PC 38:6 | 41 ± 4.4 | 820.5851 | PC 38:6 | 32.2 ± 4.5 | 14 | 17 | −21 |
| PC 38:3 | 26 ± 5.2 | 826.6321 | PC 38:3 | 26.1 ± 6.1 | 23 | 11 | 0.38 |
| PC 36:1 | 26 ± 4.6 | 802.6321 | PC 36:1 | 21.7 ± 5.2 | 24 | 17 | −16 |
| SM 36:1; O2 | 20 ± 3.7 | 745.6212 | SM 36:1; O2 | 18.5 ± 0.2 | 1.2 | 19 | −7.5 |
| SM 42:1; O2 | 20 ± 5.4 | 829.7157 | SM 42:1; O2 | 17.9 ± 1.2 | 6.4 | 20 | −10 |
| SM 40:1; O2 | 20 ± 5.1 | 801.6844 | SM 40:1; O2 | 7.2 ± 2.3 | 33 | 17 | −64 |
| SM 42:3; O2 | 17 ± 11 | 825.6844 | SM 42:3; O2 | 10.9 ± 0.7 | 6.8 | 9 | −36 |
| SM 34:2; O2* | 16 ± 2.2 | 715.5749 | SM 34:1; O2 | 4.5 ± 0.1 | 1.6 | 21 | −72 |
| PC 40:6 | 14 ± 2.6 | 848.6164 | PC 40:6 | 15.5 ± 1.7 | 11 | 19 | 11 |
| PC 32:1* | 13 ± 1.9 | 746.5695 | PC 30:0 | 16.4 ± 4.1 | 25 | 16 | 26 |
| SM 40:2; O2 | 12 ± 2.8 | 799.6688 | SM 40:2; O2 | 4.4 ± 1.5 | 33 | 13 | −63 |
| PC 36:5* | 11 ± 1.8 | 794.5695 | PC 36:4 | 7.9 ± 1.4 | 18 | 17 | −28 |
| SM 38:1; O2* | 11 ± 3.1 | 773.6531 | SM 36:1; O2 | 3.4 ± 0.2 | 5.3 | 14 | −69 |
| SM 36:2; O2* | 9.6 ± 1.5 | 743.6062 | SM 36:1; O2 | 8.2 ± 0.1 | 1.7 | 14 | −15 |
| SM 32:1; O2* | 8.4 ± 1.4 | 689.5592 | SM 34:1; O2 | 5.1 ± 0.1 | 2.2 | 15 | −39 |
| PC 32:0* | 7.2 ± 1 | 748.5851 | PC 34:1 | 6.2 ± 0.9 | 14 | 22 | −14 |
| PC 40:5 | 6.7 ± 1.1 | 850.6321 | PC 40:5 | 12.6 ± 1.5 | 12 | 39 | 88 |
| SM 34:0; O2* | 5.8 ± 1.3 | 719.6062 | SM 34:1; O2 | 15.2 ± 0.4 | 2.4 | 16 | 162 |
| SM 41:2; O2* | 5.8 ± 1.4 | 813.6844 | SM 40:1; O2 | 3.1 ± 0.5 | 15 | 18 | −46 |
| SM 38:2; O2* | 5.2 ± 1.3 | 771.6375 | SM 36:1; O2 | 6.6 ± 0.2 | 3.2 | 24 | 27 |
| SM 33:1; O2* | 4.7 ± 0.6 | 703.5749 | SM 34:1; O2 | 5.7 ± 0.1 | 1.4 | 23 | 21 |
| SM 39:1; O2* | 3.6 ± 1.0 | 787.6688 | SM 40:1; O2 | 3.3 ± 0.3 | 10 | 25 | −8.3 |
| PC 40:7* | 3.5 ± 0.8 | 846.6008 | PC 40:6 | 2.9 ± 0.4 | 14 | 27 | −17 |
| PC 40:4 | 2.9 ± 0.4 | 852.6477 | PC 40:4 | 2.5 ± 0.5 | 20 | 26 | −14 |
| PC 38:2* | 2.3 ± 0.2 | 828.6477 | PC 38:3 | 3.8 ± 1.3 | 34 | 29 | 65 |
| SM 40:3; O2* | 2.2 ± 0.79 | 797.6531 | SM 40:2; O2 | 1.1 ± 0.5 | 41 | 37 | −50 |
| PC 34:0 | 2.1 ± 0.4 | 776.6164 | PC 34:0 | 1.3 ± 0.2 | 15.4 | 24 | −38 |
| SM 36:0; O2* | 2.0 ± 0.5 | 747.6375 | SM 36:1; O2 | 7.9 ± 0.2 | 2.5 | 25 | 295 |
| PC 30:0 | 1.6 ± 0.3 | 720.5538 | PC 30:0 | 2.6 ± 1.3 | 50 | 39 | 62 |
| SM 36:3; O2* | 1.3 ± 0.41 | 741.5905 | SM 36:1; O2 | 0.6 ± 0.2 | 26 | 31 | −54 |
| SM 37:1; O2* | 1.0 ± 0.2 | 759.6375 | SM 36:1; O2 | 4.9 ± 0.1 | 2.0 | 24 | 390 |
| SM 43:2; O2* | 1.0 ± 0.3 | 841.7157 | SM 42:1; O2 | 0.7 ± 0.1 | 11 | 25 | −30 |
| SM 41:3; O2* | 0.77 ± 0.30 | 811.6688 | SM 40:1; O2 | 4.8 ± 0.4 | 7.5 | 25 | 523 |
| PC 40:8* | 0.73 ± 0.20 | 844.5851 | PC 40:6 | 3.0 ± 0.6 | 20 | 28 | 311 |
| SM 32:2; O2* | 0.66 ± 0.24 | 687.5436 | SM 34:1; O2 | 0.2 ± 0.1 | 44 | 36 | −70 |
| SM 38:3; O2* | 0.61 ± 0.24 | 769.6218 | SM 36:1; O2 | 0.5 ± 0.2 | 34 | 39 | −18 |
| SM 39:2; O2* | 0.61 ± 0.16 | 785.6531 | SM 40:1; O2 | 2.8 ± 0.2 | 8.5 | 28 | 359 |
| SM 35:2; O2* | 0.52 ± 0.21 | 729.5905 | SM 34:1; O2 | 0.8 ± 0.1 | 2.7 | 29 | 54 |

TABLE 6
Summary of 84 annotated serum PLs measured by
MSI-NACE-MS when comparing high-dose FO/placebo
post-treatment relative to baseline in pooled sub-groups.

| Lipid | | Fold- | |
|---|---|---|---|
| Identification | m/z:RMT:mode^{a} | change^{b} | p-value^{c} |
| FA 20:5 | 301.217:0.993:n | 4.16 | 0.00133 |
| FA 22:6 | 327.233:1.019:n | 3.51 | 0.00112 |
| PC 36:5 | 794.570:0.984:p | 4.48 | 0.00599 |
| PC 36:5* | 838.560:0.704:n | 1.67 | 0.0331 |
| PC 38:5 | 822.601:0.992:p | 1.49 | 0.0209 |
| PC 38:5* | 866.592:0.705:n | 1.81 | 0.0267 |
| PC 40:5* | 894.623:0.711:n | 2.05 | 0.0602 |
| PC 40:6 | 848.616:1.002:p | 1.33 | 0.0409 |
| PC 40:6* | 892.607:0.708:n | 1.54 | 0.0258 |
| PC 38:6 | 820.585:0.989:p | 1.33 | 0.0482 |
| PC 38.6* | 864.576:0.707:n | 1.54 | 0.0258 |
| PC 40:8 | 838.632:0.994:p | 1.60 | 0.0512 |
| PC 39:7 | 832.585:0.994:p | 1.48 | 0.0526 |
| PC 35:4 | 782.606:0.983:p | 0.64 | 0.0527 |
| FA 16:1 | 253.217:0.993:n | 0.68 | 0.0532 |
| PC 34:3 | 770.570:0.982:p | 0.63 | 0.0545 |
| FA 20:3 | 305.249:0.972:n | 0.60 | 0.0549 |
| PC 36:3 | 798.601:0.987:p | 0.46 | 0.0553 |
| LPE 22:6 | 524.278:0.782:n | 1.60 | 0.0581 |
| PC 34:2 | 772.585:0.983:p | 0.83 | 0.0584 |
| FA 17:1 | 267.233:0.988:n | 0.71 | 0.0589 |
| PC 38:7 | 818.570:0.986:p | 1.35 | 0.0592 |
| PC | 864.684:1.016:p | 0.77 | 0.0595 |
| O-42:5/P-42:4 | | | |
| FA 18:2 | 279.233:0.981:n | 0.67 | 0.0596 |
| PC 37:4 | 810.601:0.988:p | 0.75 | 0.0599 |
| FA 20:1 | 309.280:0.966:n | 0.64 | 0.0604 |
| PC 36:4 | 796.585:0.985:p | 0.77 | 0.0616 |
| PI 40:6 | 909.550:0.992:n | 2.21 | 0.0620 |
| PC 40:4 | 852.648:1.005:p | 0.78 | 0.0636 |
| PI 40:7 | 907.534:0.996:n | 0.61 | 0.0651 |
| PC 38:4 | 824.616:0.994:p | 0.74 | 0.0659 |
| FA 18:3 | 277.217:0.982:n | 0.80 | 0.0676 |
| PC 39:4 | 836.616:1.004:p | 1.36 | 0.0677 |
| FA 18:0 | 283.264:0.978:n | 0.82 | 0.0681 |
| PC 36:6 | 792.554:0.983:p | 1.39 | 0.0685 |
| FA 16:0 | 255.233:0.993:n | 0.72 | 0.0700 |
| PC 35:3 | 784.622:0.985:p | 0.71 | 0.0746 |
| FA 12:0 | 199.170:1.027:n | 0.86 | 0.0768 |
| PC 33:3 | 756.554:0.978:p | 0.73 | 0.0786 |
| PC 38:3 | 826.632:0.999:p | 0.65 | 0.0797 |
| PC 35:5 | 780.590:0.979:p | 0.78 | 0.0827 |
| PC 40:5 | 850.632:1.004:p | 1.15 | 0.0858 |
| PC 37:5 | 808.585:0.987:p | 0.86 | 0.0865 |
| PC 37:3 | 812.616:0.993:p | 0.76 | 0.0875 |
| PC 30:0 | 720.554:0.973:p | 1.47 | 0.0894 |
| PC 37:6 | 806.570:0.986:p | 1.26 | 0.0950 |
| FA 17:0 | 269.249:0.982:n | 0.85 | 0.107 |
| FA 20:4 | 303.233:0.993:n | 0.84 | 0.114 |
| LPE 20:5 | 498.263:0.782:n | 1.50 | 0.122 |
| FA 14:1 | 225.186:1.009:n | 0.79 | 0.123 |
| PC 35:1 | 788.653:0.987:p | 1.20 | 0.133 |
| LPE 22:5 | 526.294:0.783:n | 0.71 | 0.136 |
| PC | 862.668:1.008:p | 0.86 | 0.138 |
| O-42:6/P-42:5 | | | |
| PC 33:0 | 762.601:0.984:p | 1.28 | 0.140 |
| FA 18:1 | 281.249:0.978:n | 0.83 | 0.144 |
| PC 36:2 | 800.616:0.992:p | 0.92 | 0.144 |
| PE 38:5 | 764.524:0.763:n | 0.49 | 0.149 |
| PC 34:1 | 774.601:0.985:p | 0.90 | 0.153 |
| PC 33:2 | 758.570:0.980:p | 0.84 | 0.153 |
| PC 31:1 | 732.554:0.975:p | 1.20 | 0.184 |
| PC 31:0 | 734.570:0.978:p | 1.16 | 0.203 |
| PC 34:4 | 768.554:0.978:p | 0.76 | 0.204 |
| PC 40:7 | 846.601:0.998:p | 0.92 | 0.215 |
| LPC 22:6 | 626.346:0.701:n | 0.89 | 0.219 |
| PC 32:0 | 748.585:0.980:p | 1.14 | 0.234 |
| PC 35:2 | 786.637:0.987:p | 0.94 | 0.239 |
| PE 37:6 | 748.492:0.765:n | 0.58 | 0.291 |
| FA 14:0 | 227.202:1.005:n | 0.60 | 0.296 |
| FA 15:0 | 241.217:0.999:n | 0.85 | 0.296 |
| PC 32:2 | 744.554:0.976:p | 0.93 | 0.314 |
| FA 22:5 | 329.249:0.97:n | 0.57 | 0.320 |
| PC 39:5 | 834.601:1.000:p | 1.12 | 0.324 |
| PE 39:7 | 774.508:0.766:n | 1.65 | 0.330 |
| FA 22:4 | 331.264:0.968:n | 0.57 | 0.335 |
| PC 32:1 | 746.570:0.978:p | 0.97 | 0.399 |
| PC 34:0 | 776.616:0.985:p | 0.98 | 0.413 |
| PC 36:1 | 802.632:0.990:p | 1.02 | 0.414 |
| PC 33:1 | 760.585:0.983:p | 0.98 | 0.445 |
| PC 36:0 | 804.648:0.994:p | 1.01 | 0.461 |
| PE 38:6 | 762.508:0.764:n | 0.92 | 0.593 |
| PE 35:5 | 722.477:0.764:n | 1.39 | 0.628 |
| FA 20:2 | 307.264:0.967:n | 0.93 | 0.643 |
| LPC 20:5 | 630.331:0.703:n | 1.07 | 0.726 |
| PE 37:5 | 750.508:0.764:n | 0.96 | 0.864 |

^{a}Serum lipid extracts were analyzed by MSI-NACE-MS following methylation under positive ion mode (PCs), or underivatized under negative ion mode conditions (FAs, LPCs, PEs, LPEs, PIs). Top-ranked PCs (*) were also replicated in negative ion mode.
^{b}Average fold-change in ion response for serum lipid following FO supplementation to baseline in pooled samples.
^{c}Statistical significance of pooled serum lipid increase following FO supplementation using paired student's t-test.

TABLE 7
Responsive serum methylated PCs associated with high-dose FO
intake as classified by a repeat measures two-way mixed-model
ANOVA when comparing FO and placebo (SO) treatment arms.

| | Within-Subject Effects | | | | Between-Subject Effects | | | |
|---|---|---|---|---|---|---|---|---|
| | | | Effect | Study | | | Effect | Study |
| Serum PCs | F | p value^{a} | Size^{b} | Power | F | p value | Size^{b} | Power |
| PC 36:5 + PC | 6.89 | 7.81 × | 0.346 | 0.965 | 74.1 | 9.93 × | 0.851 | 1.000 |
| 38:6^{c} | | 10⁻⁴ | | | | 10⁻⁷ | | |
| Total EPA PCs^{d} | 6.76 | 8.83 × | 0.342 | 0.962 | 54.2 | 6.00 × | 0.806 | 1.000 |
| | | 10⁻⁴ | | | | 10⁻⁶ | | |
| PC 36:5 | 6.69 | 9.42 × | 0.340 | 0.960 | 33.4 | 6.30 × | 0.720 | 1.000 |
| | | 10⁻⁴ | | | | 10⁻⁵ | | |
| Total omega-3 | 6.30 | 1.37 × | 0.326 | 0.949 | 14.8 | 2.00 × | 0.533 | 0.945 |
| PCs^{e} | | 10⁻³ | | | | 10⁻³ | | |
| Total DHA PCs^{f} | 5.39 | 3.36 × | 0.293 | 0.909 | 32.8 | 7.00 × | 0.716 | 1.000 |
| | | 10⁻³ | | | | 10⁻⁵ | | |
| PC 38:5 | 5.12 | 4.43 × | 0.282 | 0.893 | 39.0 | 3.00 × | 0.750 | 1.000 |
| | | 10⁻³ | | | | 10⁻⁵ | | |
| PC 38:6 | 4.94 | 5.27 × | 0.276 | 0.882 | 20.2 | 6.01 × | 0.609 | 1.000 |
| | | 10⁻³ | | | | 10⁻⁴ | | |
| PC 40:6 | 4.51 | 8.26 × | 0.258 | 0.848 | 25.6 | 2.17 × | 0.664 | 0.997 |
| | | 10⁻³ | | | | 10⁻⁴ | | |
| PC 36:1 | 1.37 | 2.66 × | 0.095 | 0.336 | 0.01 | 9.35 × | 0.001 | 0.051 |
| | | 10⁻³ | | | | 10⁻¹ | | |
| PC 36:2 | 0.23 | 8.72 × | 0.018 | 0.090 | 0.16 | 6.95 × | 0.012 | 0.066 |
| | | 10⁻¹ | | | | 10⁻¹ | | |

TABLE 8
Clinical characteristics of participants in a placebo-controlled EPA
or DHA (3.0 g/day) treatment intervention over a 90-day period.

| | | Clinical Characteristic | | | |
|---|---|---|---|---|---|
| Grouping | Sex | Age | BMI | O3I (Baseline) | O3I (Post) |
| Placebo | Female, n = 14 | 21 ± 1 | 24.6 ± 3.3 | 3.98 ± 0.73 | 3.76 ± 0.60 |
| (OO) | Male, n = 13 | 21 ± 2 | 24.0 ± 4.0 | 3.46 ± 0.77 | 3.45 ± 0.59 |
| EPA | Female, n = 14 | 21 ± 2 | 22.5 ± 2.8 | 3.61 ± 0.64 | 6.75 ± 1.20 |
| | Male, n = 14 | 21 ± 2 | 23.4 ± 2.7 | 3.40 ± 0.87 | 6.23 ± 1.10 |
| DHA | Female, n = 15 | 22 ± 2 | 22.7 ± 2.7 | 3.73 ± 0.51 | 8.04 ± 1.34 |
| | Male, n = 13 | 23 ± 3 | 24.7 ± 3.8 | 3.15 ± 0.64 | 8.73 ± 0.97 |

TABLE 9
Top-ranked plasma PCs and their panels that were associated
with O3I following high-dose EPA or DHA supplementation
as compared to OO as placebo. Plasma PC responses were
reported using their relative peak areas.

| | | Pearson | |
|---|---|---|---|
| | Number of Lipids | Correlation | |
| Lipid Panel^{a} | (EPA, DHA) | (r) | p-value |
| PC 36:5 + PC 38:6^{b} | 2 (EPA: 1, DHA: 1) | 0.764 | 3.0 × 10⁻³³ |
| PC 36:5 + PC 38:5^{c} + | 3 (EPA: 1, DHA: 2) | 0.711 | 5.6 × 10⁻²⁷ |
| PC 38:6 + PC 40:6 | | | |
| PC 36:5 + PC 38:5^{c} + | 2 (EPA: 1, DHA: 1) | 0.706 | 1.7 × 10⁻²⁶ |
| PC 38:6 | | | |
| PC 38:6 | 1 (EPA: 0, DHA: 1) | 0.663 | 1.5 × 10⁻²² |
| PC 36:5 + PC 38:5^{c} + | 5 (EPA: 2, DHA: 3) | 0.636 | 2.5 × 10⁻²⁰ |
| PC 40:5 + PC 36:6 + | | | |
| PC 38:6 + PC 40:6 | | | |
| PC 36:5 | 1 (EPA: 1, DHA: 0) | 0.494 | 1.2 × 10⁻¹¹ |
| PC 38:5^{c} | 0 (EPA: 0, DHA: 0) | 0.484 | 3.5 × 10⁻¹¹ |
| PC 40:6 | 1 (EPA: 0, DHA: 1) | 0.387 | 2.5 × 10⁻⁷ |
| PC 36:6 | 1 (EPA: 0, DHA: 1) | 0.225 | 3.5 × 10⁻³ |
| PC 40:5 | 1 (EPA: 1, DHA: 0) | 0.222 | 4.0 × 10⁻³ |

^{a}All PCs are derivatized as their cationic phosphomethylesters to improve separation resolution and ionization responses in MSI-NACE-MS under positive ion mode detection.
^{b}PC 36:5 = PC (16:0_20:5), PC 38:6 = PC (16:0_22:6).
^{c}PC 38:5 was subsequently determined to be comprised of two unresolved lipid species, namely PC 16:0_22:5 and PC 18:1_20:4.

TABLE 10
Sex-dependence of EPA or DHA treatment intervention on changes in
plasma PC 36:5 + PC 38:6 and erythrocyte PL membrane derived O3I.

| | | | Δ(PC 36:5 + | | ΔPC | |
|---|---|---|---|---|---|---|
| Treatment | Sex | Age^{a} | PC 38:6)^{a} | ΔO3I^{a} | panel^{b} | ΔO3I^{c} |
| Placebo | F = 14 | 21.0 ± 1.4 | 0.43 ± 11.38 | −0.22 ± 0.54 | 0.738 | 6.16 × |
| (OO) | M = 13 | 20.9 ± 2.2 | 1.74 ± 8.51 | −0.01 ± 0.65 | | 10⁻¹ |
| EPA | F = 14 | 21.0 ± 2.1 | 33.71 ± 16.15 | 3.14 ± 1.25 | 1.89 × | 5.71 × |
| | M = 14 | 21.6 ± 2.0 | 19.30 ± 14.26 | 2.83 ± 1.14 | 10⁻² | 10⁻¹ |
| DHA | F = 15 | 21.5 ± 1.8 | 26.11 ± 16.15 | 4.31 ± 1.26 | 9.66 × | 1.90 × |
| | M = 13 | 22.6 ± 2.8 | 25.80 ± 20.88 | 5.57 ± 1.10 | 10⁻¹ | 10⁻² |

^{a}Reported values are mean ± standard deviation
^{b}p-values calculated with Student's t-test after confirming normality using Shapiro-Wilk test (p > 0.05)
^{c}p-values calculated with Mann-Whitney U test after confirming non-normality using Shapiro-Wilk test (p < 0.05)

### CITATIONS

(1) Quehenberger, O.; Dennis, E. A. N. Engl. J. Med. 2011, 365 (19), 1812-1823.
(2) Shevchenko, A.; Simons, K. Nat. Rev. Mol. Cell Bio. 2010, 11 (8), 593-598.
(3) Meikle, T. G.; Huynh, K.; Giles, C.; Meikle, P. J. J. Lipid Res. 2021, 62, 100127.
(4) Han, X. Nat. Rev. Endocrinol. 2016, 12 (11), 668-679.
(5) Hyötyläinen, T.; Bondia-Pons, I.; Orešič, M. Mol. Nutr. Food Res. 2013, 57 (8), 1306-1318.
(6) Azab, S. M.; Souza, R. J. de; Teo, K. K.; Anand, S. S.; Williams, N. C.; Holzschuher, J.; McGlory, C.; Philips, S. M.; Britz-Mckibbin, P. J. Lipid Res. 2020, 61 (6), 933-944.
(7) Han, X.; Gross, R. W. J. Lipid Res. 2003, 44 (6), 1071-1079.
(8) Kohno, S.; Keenan, A. L.; Ntambi, J. M.; Miyazaki, M. Biochem. Biophys. Res. Comm. 2018, 504 (3), 590-595.
(9) Rustam, Y. H.; Reid, G. E. Anal. Chem. 2017, 90 (1), 374-397.
(10) McDonald, J. G. et al. Nat. Metabolism 2022, 4 (9), 1086-1088.
(11) O'Donnell, V. B.; FitzGerald, G. A.; Murphy, R. C.; Liebisch, G.; Dennis, E. A.; Quehenberger, O.; Subramaniam, S.; Wakelam, M. J. O. Circul. Genom. Precis. Med. 2020, 13 (6), e003019-e003019.
(12) Drotleff, B.; Lämmerhofer, M. Anal. Chem. 2019, 91 (15), 9836-9843.
(13) Tsugawa, H.; Ikeda, K.; Takahashi, M.; Satoh, A.; Mori, Y.; Uchino, H.; Okahashi, N.; Yamada, Y.; Tada, I.; Bonini, P.; Higashi, Y.; Okazaki, Y.; Zhou, Z.; Zhu, Z. J.; Koelmel, J.; Cajka, T.; Fiehn, O.; Saito, K.; Arita, M.; Arita, M. A. Nat. Biotechnol. 2020, 38 (10), 1159-1163.
(14) Delabriere, A.; Warmer, P.; Brennsteiner, V.; Zamboni, N. Anal. Chem. 2021, 93 (45), 15024-15032.
(15) Bowden, J. A. et al. J. Lipid Res. 2017, 58 (12), 2275-2288.
(16) Initiative, A. D. N.; Consortium, A. D. M.; Barupal, D. K.; Fan, S.; Wancewicz, B.; Cajka, T.; Sa, M.; Showalter, M. R.; Baillie, R.; Tenenbaum, J. D.; Louie, G.; Alzheimer's Disease Neuroimaging Initiative; Alzheimer's Disease Metabolomics Consortium; Kaddurah-Daouk, R., Fiehn, O. Sci. Data 2018, 5 (1), 180263.
(17) Thompson, J. W. et al. Anal. Chem. 2019, 91 (22), 14407-14416.
(18) Züllig, T.; Trötzmüller, M.; Köfeler, H. C. Anal. Bioanal. Chem. 2020, 412 (10), 2191-2209.
(19) Abel, K.; deSchmertzing, H.; Peterson, J. I. J. Bacteriol. 1963, 85 (5), 1039-1044.
(20) Han, X.; Gross, R. W. J. Lipid Res. 2021, 63 (2), 100164.
(21) Han, X.; Gross, R. W. Mass Spectrom. Rev. 2005, 24 (3), 367-412.
(22) Xu, T.; Hu, C.; Xuan, Q.; Xu, G. Anal. Chim. Acta 2020, 1137, 156-169.
(23) Cajka, T.; Fiehn, O. Methods Mol. Biol. 2017, 1609, 149-170.
(24) Lange, M.; Ni, Z.; Criscuolo, A.; Fedorova, M. Chromatographia 2019, 82 (1), 77-100.
(25) Harrieder, E.-M.; Kretschmer, F.; Böcker, S.; Witting, M. J. Chromatogr. B 2021, 1188, 123069.
(26) Narváez-Rivas, M.; Zhang, Q. J. Chromatogr. A 2016, 1440, 123-134.
(27) Plumb, R. S.; Isaac, G.; Rainville, P. D.; Hill, J.; Gethings, L. A.; Johnson, K. A.; Lauterbach, J.; Wilson, I. D. J. Proteome Res 2022, 21 (3), 691-701.
(28) Sorensen, M. J.; Miller, K. E.; Jorgenson, J. W.; Kennedy, R. T. J. Chromatogr. A 2020, 1611, 460575.
(29) Baglai, A.; Gargano, A. F. G.; Jordens, J.; Mengerink, Y.; Honing, M.; Wal, S. van der; Schoenmakers, P. J. J. Chromatogr. A 2017, 1530, 90-103.
(30) Wolrab, D.; Chocholoušková, M.; Jirásko, R.; Peterka, O.; Holčapek, M. Anal. Bioanal. Chem. 2020, 412 (10), 2375-2388.
(31) Leaptrot, K. L.; May, J. C.; Dodds, J. N.; McLean, J. A. Nat. Commun. 2019, 10 (1), 985.
(32) Lee, J.-H.; Kim, S.-J.; Lee, S.; Rhee, J.-K.; Lee, S. Y.; Na, Y.-C. Anal. Chim. Acta 2017, 984, 223-231.
(33) Sándor, V.; Berkics, B. V.; Kilár, A.; Kocsis, B.; Kilár, F.; Dörnyei, Á. Electrophoresis 2020, 41 (13-14), 1178-1188.
(34) Gao, F.; Zhang, Z.; Fu, X.; Li, W.; Wang, T.; Liu, H. Electrophoresis 2007, 28 (9), 1418-1425.
(35) Montealegre, C.; Sánchez-Hernández, L.; Crego, A. L.; Marina, M. L. J. Agr. Food Chem. 2013, 61 (8), 1823-1832.
(36) Azab, S.; Ly, R.; Britz-McKibbin, P. Anal. Chem. 2019, 91 (3), 2329-2336.
(37) Azab, S. M.; Souza, R. J. de; Ly, R.; Teo, K. K.; Atkinson, S. A.; Morrison, K. M.; Anand, S. S.; Britz-Mckibbin, P. Prostagl. Leukot. Essent. Fatty Acids 2022, 176, 102378.
(38) Ly, R.; Ly, N.; Sasaki, K.; Suzuki, M.; Kami, K.; Ohashi, Y.; Britz-McKibbin, P. J. Proteome Res. 2022, 21 (3), 768-777.
(39) Shanmuganathan, M.; Kroezen, Z.; Gill, B.; Azab, S.; Souza, R. J. de; Teo, K. K.; Atkinson, S.; Subbarao, P.; Desai, D.; Anand, S. S.; Britz-Mckibbin, P. Nat. Protoc. 2021, 16 (4), 1966-1994.
(40) DiBattista, A.; McIntosh, N.; Lamoureux, M.; Al-Dirbashi, O. Y.; Chakraborty, P.; Britz-McKibbin, P. Anal. Chem. 2017, 89 (15), 8112-8121.
(41) Xia, F.; Wan, J. Mass Spectrom. Rev. 2021, e21729.
(42) Wasslen, K. V.; Canez, C. R.; Lee, H.; Manthorpe, J. M.; Smith, J. C. Anal. Chem. 2014, 86 (19), 9523-9532.
(43) Wasslen, K. V.; Tan, L. H.; Manthorpe, J. M.; Smith, J. C. Anal. Chem. 2014, 86 (7), 3291-3299.
(44) Betancourt, S. K.; Canez, C. R.; Shields, S. W. J.; Manthorpe, J. M.; Smith, J. C.; McLuckey, S. A. Anal. Chem. 2017, 89 (17), 9452-9458.
(45) Dallinger, D.; Kappe, C. O. Nat. Protoc. 2017, 12 (10), 2138-2147.
(46) Yamamoto, M.; Ly, R.; Gill, B.; Zhu, Y.; Moran-Mirabal, J.; Britz-McKibbin, P. Anal. Chem. 2016, 88 (21), 10710-10719.
(47) White, E. H.; Baum, A. A.; Eitel, D. E. Org. Synth. 2003, 48, 102-105.
(48) Caperos, J. R.; Fernández, J. G. Brit. J. Ind. Med. 1977, 34 (3), 229-233.
(49) Furukawa, T.; Hinou, H.; Takeda, S.; Chiba, H.; Nishimura, S.; Hui, S. Chembiochem. 2017, 18 (19), 1903-1909.
(50) Höring, M.; Stieglmeier, C.; Schnabel, K.; Hallmark, T.; Ekroos, K.; Burkhardt, R.; Liebisch, G. Anal. Chem. 2022 94 (36): 12292-12296.
(51) Hsu, F.-F.; Turk, J. J. Am. Soc. Mass Spectr. 2000, 11 (5), 437-449.
(52) Claes, B. S. R.; Bowman, A. P.; Poad, B. L. J.; Young, R. S. E.; Heeren, R. M. A.; Blanksby, S. J.; Ellis, S. R. Anal. Chem. 2021, 93 (28), 9826-9834.
(53) Ma, X.; Zhang, W.; Li, Z.; Xia, Y.; Ouyang, Z. Acc Chem Res. 2021 54 (20): 3873-3882.
(54) Harada, S.; Hirayama, A.; Chan, Q.; Kurihara, A.; Fukai, K.; Iida, M.; Kato, S.; Sugiyama, D.; Kuwabara, K.; Takeuchi, A.; Akiyama, M.; Okamura, T.; Ebbels, T. M. D.; Elliott, P.; Tomita, M.; Sato, A.; Suzuki, C.; Sugimoto, M.; Soga, T.; Takebayashi, T. PLOS One 2018 13 (1): e0191230.
(55) Ly, R.; Macintyre, B. C; Philips, S. M.; McGlory, C.; Mutch, D. M.; Britz-McKibbin, P. J. Lipid Res. 2023 64 (11): 100445.
(56) Mente, A., M. Dehghan, S. Rangarajan, M. McQueen, G. Dagenais, A. Wielgosz, S. Lear, W. Li, H. Chen, S. Yi, et al. 2017. Association of dietary nutrients with blood lipids and blood pressure in 18 countries: a cross-sectional analysis from the PURE study. Lancet Diabetes Endocrinol. 5:774-787.
(57) Astrup A., N. Teicholz, F. Magkos, D. M. Bier, J. T. Brenna, J. C. King, A. Mente, J. M. Ordovas, J. S. Volek, S. Yusuf, and R. M. Krauss. 2021. Dietary saturated fats and health: Are the U.S. guidelines evidence-based? Nutrients 13:3305.
(58) Temple, N. J. 2018. Fat, sugar, whole grains and heart disease: 50 years of confusion. Nutrients 10:39.
(59) Surette, M. E. 2008. The science behind dietary omega-3 fatty acids. Can. Med. Assoc. J. 178:177-180.
(60) Plourde M., and S. C. Cunnane. 2007. Extremely limited synthesis of long chain polyunsaturates in adults: Implications for their dietary essentiality and use as supplements. Appl. Physiol. Nutr. Metab. 32:619-634.
(61) Rimm, E. B., L. J. Appel, S. E. Chiuve, L. Djoussé, M. B. Engler, P. M. Kris-Etherton, D. Mozaffarian, D. S. Siscovick, and A. H. Lichtenstein. 2018. Seafood long-chain n−3 polyunsaturated fatty acids and cardiovascular disease: A science advisory from the American Heart Association. Circulation 138: e35-e47.
(62) Field, C. J., and L. Robinson. 2019. Dietary fats. Adv. Nutr. 10:722-724.
(63) Calder, P. C. 2010. Omega-3 fatty acids and inflammatory processes. Nutrients. 2:355-374.
(64) Ferguson, E. J., Seigel, J. W., McGlory, C. 2021. Omega-3 fatty acids and human skeletal muscle. Curr. Opin. Clin. Nutr. Metab. Care. 24:114-119.
(65) Cladis, D. P., A. C. Kleiner, H. H. Freiser, and C. R. Santerre. 2014. Fatty acid profiles of commercially available finfish fillets in the United States. Lipids 49:1005-1018.
(66) Ryckebosch, E., C. Bruneel, R. Termote-Verhalle, K. Goiris, K. Muylaert, and I. Foubert. 2014. Nutritional evaluation of microalgae oils rich in omega-3 long chain polyunsaturated fatty acids as an alternative for fish oil. Food Chem. 160:393-400.
(67) Siscovick, D. S., T. A. Barringer, A. M. Fretts, J. H. Y. Wu, A. H. Lichtenstein, R. B. Costello, P. M. Kris-Etherton, T. A. Jacobson, M. B. Engler, H. M. Alger, et al. 2017. Omega-3 polyunsaturated fatty acid (Fish Oil) supplementation and the prevention of clinical cardiovascular disease. Circulation 135: e867-e884.
(68) Abdelhamid, A. S., T. J. Brown, J. S. Brainard, P. Biswas, G. C. Thorpe, H. J. Moore, K. H. Deane, C. D. Summerbell, H. V. Worthington, F. Song, et al. 2020. Omega-3 fatty acids for the primary and secondary prevention of cardiovascular disease. Cochrane Db. Syst. Rev. 2020: CD003177.
(69) Watanabe, Y., and I. Tatsuno. 2020. Prevention of cardiovascular events with omega-3 polyunsaturated fatty acids and the mechanism involved. J Atheroscler Thromb. 27:183-198.
(70) Miller, M., D. L. Bhatt, P. G. Steg, E. A. Brinton, T. A. Jacobson, L. Jiao, J.-C. Tardif, C. M. Ballantyne, M. Budoff, R. P. Mason, et al. 2023. Potential effects of icosapent ethyl on cardiovascular outcomes in cigarette smokers: REDUCE-IT smoking. Eur. Heart J.-Cardiovasc. Pharmacother. 9:129-137.
(71) Shojima, Y., Y. Ueno, R. Tanaka, K. Yamashiro, N. Miyamoto, K. Hira, N. Kurita, S. Nakajima, T. Urabe, and N. Hattori. 2020. Eicosapentaenoic-to-arachidonic acid ratio predicts mortality and recurrent vascular events in ischemic stroke patients. J. Atheroscler. Thromb. 27:969-977.
(72) Merino D. M., D. W. Ma, and D. M. Mutch. 2010. Genetic variation in lipid desaturases and its impact on the development of human disease. Lipids Health Dis. 9:63.
(73) Garneau, V., I. Rudkowska, A.-M. Paradis, G. Godin, P. Julien, L. Pérusse, and M.-C. Vohl. 2012. Omega-3 fatty acids status in human subjects estimated using a food frequency questionnaire and plasma phospholipids levels. Nutr. J. 11:46.
(74) Rafiq, T., S. M. Azab, K. K. Teo, L. Thabane, S. S. Anand, K. M. Morrison, R. J. de Souza, and P. Britz-Mckibbin. 2021. Nutritional metabolomics and the classification of dietary biomarker candidates: A critical review. Adv. Nutr. 12:2333-2357.
(75) Harris, W. S., and C. von Schacky. 2004. The omega-3 index: A new risk factor for death from coronary heart disease? Prev. Med. 39:212-220.
(76) Harris, W. S. 2008. The omega-3 index as a risk factor for coronary heart disease. Am J Clin Nutrition 87: 1997S-2002S.
(77) Harris, W. S., L. D. Gobbo, and N. L. Tintle. 2017. The omega-3 index and relative risk for coronary heart disease mortality: Estimation from 10 cohort studies. Atherosclerosis 262:51-54.
(78) Langlois, K., and W. M. N. Ratnayake. 2015. Omega-3 index of Canadian adults. Health Rep. 26:3-11.
(79) Baylin, A., and H. Campos. 2006. The use of fatty acid biomarkers to reflect dietary intake. Curr. Opin. Lipidol. 17:22-27.
(80) Stark, K. D., J. J. Aristizabal Henao, A. H. Metherel, and L. Pilote. 2016. Translating plasma and whole blood fatty acid compositional data into the sum of eicosapentaenoic and docosahexaenoic acid in erythrocytes. Prostaglandins Leukot. Essent. Fatty Acids. 104:1-10.
(81) Albert, C. M., H. Campos, M. J. Stampfer, P. M. Ridker, J. E. Manson, W. C. Willett, and J. Ma. 2002. Blood levels of long-chain n−3 fatty acids and the risk of sudden death. New Engl. J. Med. 346:1113-1118.
(82) Schuchardt J. P., N. Tintle, J. Westra, and W. S Harris. 2022. Estimation and predictors of the Omega-3 Index in the UK Biobank. Br J Nutr. 10:1-40.
(83) Stark, K. D. 2008. Analytical implications of routine clinical testing for omega-3 fatty acid biomarkers. Lipid Technol. 20:177-179.
(84) McGlory, C., S. H. M. Gorissen, M. Kamal, R. Bahniwal, A. J. Hector, S. K. Baker, A. Chabowski, and S. M. Phillips. 2019. Omega-3 fatty acid supplementation attenuates skeletal muscle disuse atrophy during two weeks of unilateral leg immobilization in healthy young women. FASEB J. 33:4586-4597.
(85) Klingel, S. L., A. H. Metherel, M. Irfan, A. Rajna, A. Chabowski, R. P. Bazinet, and D. M. Mutch. 2019. EPA and DHA have divergent effects on serum triglycerides and lipogenesis, but similar effects on lipoprotein lipase activity: A randomized controlled trial. Am. J. Clin. Nutr. 110:1502-1509.
(86) Azab, S. M., R. J. de Souza, R. Ly, K. K. Teo, S. A. Atkinson, K. M. Morrison, S. S. Anand, and P. Britz-Mckibbin. 2022. Non-esterified fatty acids as biomarkers of diet and glucose homeostasis in pregnancy: The impact of fatty acid reporting methods NEFA reporting methods affect dietary and cardiometabolic endpoints. Prostaglandins Leukot. Essent. Fat Acids 176:102378.
(87) Azab, S. M., R. J. de Souza, K. K. Teo, (for the FAMILY investigators), S. S. Anand, N. C. Williams, J. Holzschuher, C. McGlory, S. M. Philips, and P. Britz-Mckibbin. 2020. Serum nonesterified fatty acids have utility as dietary biomarkers of fat intake from fish, fish oil, and dairy in women. J. Lipid Res. 61:933-944.
(88) Ly, R., N. Ly, K. Sasaki, M. Suzuki, K. Kami, Y. Ohashi, and P. Britz-McKibbin. 2022. Nontargeted serum lipid profiling of nonalcoholic steatohepatitis by multisegment injection-nonaqueous capillary electrophoresis-mass spectrometry: A multiplexed separation platform for resolving ionic lipids. J Proteome Res. 21:768-777.
(89) Ly R, Torres L C, Ly N, Britz-McKibbin P. Expanding Lipidomic Coverage in Multisegment Injection-Nonaqueous Capillary Electrophoresis-Mass Spectrometry via a Convenient and Quantitative Methylation Strategy. Anal Chem. 2023 Dec. 5; 95 (48): 17513-17524.
(90. Folch, J., M. Lees, and G. H. S. Stanley. 1957. A simple method for the isolation and purification of total lipides from animal tissues. J Biol. Chem. 226:497-509.
(91) Metherel A. H., M. Irfan, S. L. Klingel, D. M. Mutch, and R. P. Bazinet. 2019. Compound-specific isotope analysis reveals no retroconversion of DHA to EPA but substantial conversion of EPA to DHA following supplementation: A randomized control trial. Am. J. Clin. Nutr. 110:823-831.
(92) Klingel S. L., A. H. Metherel, M. Irfan, A. Rajna, A. Chabowski, R. P. Bazinet, and D. M Mutch. 2019. EPA and DHA have divergent effects on serum triglycerides and lipogenesis, but similar effects on lipoprotein lipase activity: a randomized controlled trial. Am. J. Clin. Nutr. 110:1502-1509.
(93) Yamamoto, M., M. I. Pinto-Sanchez, P. Bercik, and P. Britz-McKibbin. 2019 Metabolomics reveals elevated urinary excretion of collagen degradation and epithelial cell turnover products in irritable bowel syndrome patients. Metabolomics 15:82.
(94) Bowden J. A., A. Heckert, C. Z. Ulmer, C. M. Jones, J. P. Koelmel, L. Abdullah, L. Ahonen, Y. Alnouti, A. M. Armando, J. M. Asara, et al. 2017. Harmonizing lipidomics: NIST interlaboratory comparison exercise for lipidomics using SRM 1950-metabolites in frozen human plasma. J. Lipid Res. 58:2275-2288.
(95) Pang, Z., J. Chong, G. Zhou, D. A. de Lima Morais, L. Chang, M. Barrette, C. Gauthier, P. É. Jacques, S. Li, and J. Xia. 2021. MetaboAnalyst 5.0: Narrowing the gap between raw spectra and functional insights. Nucleic Acids Res. 49: W388-W396.
(96) Azab, S., R. Ly, and P. Britz-Mckibbin. 2019. Robust method for high-throughput screening of fatty acids by multisegment injection-nonaqueous capillary electrophoresis-mass spectrometry with stringent quality control. Anal. Chem. 91:2329-2336.
(97) Kuehnbaum, N. L., A. Kormendi, and P. Britz-McKibbin. 2013. Multisegment injection-capillary electrophoresis-mass spectrometry: A high-throughput platform for metabolomics with high data fidelity. Anal. Chem. 85:10664-10669.
(98) Canez, C. R., S. W. Shields, M. Bugno, K. V. Wasslen, H. P. Weinert, W. G. Willmore, J. M. Manthorpe, and J. C. Smith. 2016. Trimethylation enhancement using (13)C-diazomethane ((13)C-TrEnDi): Increased sensitivity and selectivity of phosphatidylethanolamine, phosphatidylcholine, and phosphatidylserine lipids derived from complex biological samples. Anal. Chem. 88:6996-7004.
(99) Dyerberg J. 1989. Coronary heart disease in Greenland Inuit: A paradox. Implications for western diet patterns. Arctic Med. Res. 48:47-54.
(100) Proust F., M. Lucas, and E. Dewailly 2014. Fatty acid profiles among the Inuit of Nunavik: Current status and temporal change. Prostaglandins Leukot Essent Fatty Acids. 90:159-167.
(101) Skogli H. R., D. Geoffroy, H. A. Weiler, G. S. Tell, L. J. Kirmayer, and G. M. Egeland 2017. Associations between omega-3 fatty acids and 25 (OH) D and psychological distress among Inuit in Canada. Int J Circumpolar Health 76:1302684.
(102) Daviglus M. L., J. Stamler, A. J. Orencia, A. R. Dyer, K. Liu, P. Greenland, M. K. Walsh, D. Morris, and R. B. Shekelle 1997. Fish consumption and the 30-year risk of fatal myocardial infarction. N Engl J Med. 336:1046-1053.
(103) Del Gobbo L. C., F. Imamura, S. Aslibekyan, M. Marklund, J. K. Virtanen, M. Wennberg et al. 2016. w-3 polyunsaturated fatty acid biomarkers and coronary heart disease: Pooling project of 19 cohort studies. JAMA Intern. Med. 176:1155-1166.
(104) Micha R., J. L. Peñalvo, F. Cudhea, F. Imamura, C. D. Rehm, and D. Mozaffarian 2017. Association between dietary factors and mortality from heart disease, stroke, and type 2 diabetes in the United States. JAMA 317:912-924.
(105) McBurney M. I., N. L. Tintle, R. S. Vasan, A. Sala-Vila, and W. S. Harris 2021. Using an erythrocyte fatty acid fingerprint to predict risk of all-cause mortality: The Framingham Offspring Cohort. Am. J. Clin. Nutr. 114:1447-1454.
(106) Kleber M. E., G. E. Delgado, S. Lorkowski, W. März, and C. von Schacky 2016. Omega-3 fatty acids and mortality in patients referred for coronary angiography. The Ludwigshafen Risk and Cardiovascular Health Study. Atherosclerosis 252:175-181.
(107) Yokoyama, M., H. Origasa, M. Matsuzaki, Y. Matsuzawa, Y. Saito, Y. Ishikawa, S. Oikawa, J. Sasaki, H. Hishida, H. Itakura, et al. 2007. Effects of eicosapentaenoic acid on major coronary events in hypercholesterolaemic patients (JELIS): A randomised open-label, blinded endpoint analysis. Lancet 369:1090-1098.
(108) Bhatt D. L., P. G. Steg, M. Miller, E. A. Brinton, T. A. Jacobson, S. B. Ketchum, R. T. Doyle, R. A. Juliano, L. Jiao, C. Granowitz, et al. 2019. Cardiovascular risk reduction with icosapent ethyl for hypertriglyceridemia. New Engl. J. Med. 380:11-22.
(109) Singh S., Arora R. R., Singh M., and Khosla S. 2016. Eicosapentaenoic acid versus docosahexaenoic acid as options for vascular risk prevention: A fish story. Am J Ther. 23: e905-e910.
(110) Allam-Ndoul B., F. Guenard, O. Barbier, and M. C. Vohl. 2017. A study of the differential effects of eicosapentaenoic acid (EPA) and docosahexaenoic acid (DHA) on gene expression profiles of stimulated Thp-1 macrophages. Nutrients 9:424.
(111) Sherratt, S. C. R., R. A. Juliano, C. Copland, D. L. Bhatt, P. Libby, and R. P. Mason. 2021. EPA and DHA containing phospholipids have contrasting effects on membrane structure. J. Lipid Res. 62:100106.
(112) Chang W. C., J. So, and S. Lamon-Fava. 2021. Differential and shared effects of eicosapentaenoic acid and docosahexaenoic acid on serum metabolome in subjects with chronic inflammation. Sci. Rep. 11:16324.
(113) Calder P. C. 2015. Functional roles of fatty acids and their effects on human health. JPEN J. Parenter. Enteral Nutr. 39: 18S-32S.
(114) Stark K. D., M. E. Van Elswyk, M. R. Higgins, C. A. Weatherford, and N. Salem Jr. 2016. Global survey of the omega-3 fatty acids, docosahexaenoic acid and eicosapentaenoic acid in the blood stream of healthy adults. Prog. Lipid Res. 63:132-152.
(115) Brenna, J. T., M. Plourde, K. D. Stark, P. J. Jones, and Y.-H. Lin. 2018. Best practices for the design, laboratory analysis, and reporting of trials involving fatty acids. Am. J. Clin. Nutr. 108:211-227.
(116) Schuchardt J. P., N. Tintle, J. Westra, and W. S. Harris. 2022. Estimation and predictors of the Omega-3 Index in the UK Biobank. Br. J. Nutr. 10:1-40.
(117) Züllig, T., M. Trötzmüller, and H. C. Köfeler. 2020. Lipidomics from sample preparation to data analysis: A primer. Anal. Bioanal. Chem. 412:2191-2209.
(118) Shields, S. W. J., C. A. Rosales, J. A. Roberts, P. J. Pallister, K. V. Wasslen, J. M. Manthorpe and J. C. Smith 2021. iTrEnDi: In situ trimethylation enhancement using diazomethane: Improved and expanded glycerophospholipid and sphingolipid analyses via a microscale autonomous derivatization platform. Anal. Chem. 93:1084-1091.
(119) Höring, M., C. S. Ejsing, S. Krautbauer, V. M. Ertl, R. Burkhardt and G. Liebisch. 2021. Accurate quantification of lipid species affected by isobaric overlap in Fourier-transform mass spectrometry. J. Lipid Res. 62:100050.
(120) Dawczynski, C., J. Plagge, G. Jahreis, G. Liebisch, M. Höring, C. Seeliger, and J. Ecker. 2022. Dietary PUFA preferably modify ethanolamine-containing glycerophospholipids of the human plasma lipidome. Nutrients 14:3055.
(121) Giltay E. J., L. J. Gooren, A. W. Toorians, M. B. Katan, and P. L. Zock. 2004. Docosahexaenoic acid concentrations are higher in women than in men because of estrogenic effects. Am. J. Clin. Nutr. 80:1167-1174.
(122) Zhang, Z., V. L. Fulgoni, P. M. Kris-Etherton, and S. H. Mitmesser. 2018. Dietary intakes of EPA and DHA omega-3 fatty acids among US childbearing-age and pregnant women: An analysis of NHANES 2001-2014. Nutrients 10:416.
(123) Carlson S. E., B. J. Gajewski, C. J.Valentine, E. H. Kerling, C. P. Weiner, M. Cackovic, C. S. Buhimschi, L. K. Rogers, S. A. Sands, A. R. Brown, D. P. Mudaranthakam, S. A. Crawford, and E. A. DeFranco. 2021. Higher dose docosahexaenoic acid supplementation during pregnancy and early preterm birth: A randomised, double-blind, adaptive-design superiority trial. EClinicalMedicine. 36:100905.
(124) Rein, D., M. Claus, W. Frosch, W. März, S. Lorkowski, S. Webendoerfer, and T. Schreiner. 2020 Changes in erythrocyte omega-3 fatty acids in German employees upon dietary advice by corporate health. Nutrients 12:3267.
(125) Jackson K. H., J. M. Polreis, N. L. Tintle, P. M. Kris-Etherton, and W. S. Harris. 2019. Association of reported fish intake and supplementation status with the omega-3 index. Prostaglandins Leukot. Essent. Fatty Acids 142:4-10.
(126) J. Allaire, Harris W. S., Vors C., Charest A., Marin J., Jackson K. H., Tchernof A., Couture P., and Lamarche B. 2017. Supplementation with high-dose docosahexaenoic acid increases the omega-3 index more than high-dose eicosapentaenoic acid. Prostaglandins Leukot. Essent. Fatty Acids. 120:8-14.
(127) Walker, R. E., K. H. Jackson, N. L. Tintle, G. C. Shearer, A. Bernasconi, S. Masson, R. Latini, B. Heydari, R. Y. Kwong, M. Flock, et al. 2019. Predicting the effects of supplemental EPA and DHA on the omega-3 index. Am. J. Clin. Nutr. 110:1034-1040.
(128) Minihane A. M. 2016. Impact of genotype on EPA and DHA status and responsiveness to increased intakes. Nutrients 8:123.
(129) Iwamatsu K., S. Abe, H. Nishida, M. Kageyama, T. Nasuno, M. Sakuma, S. Toyoda, and T. Inoue. 2016. Which has the stronger impact on coronary artery disease, eicosapentaenoic acid or docosahexaenoic acid? Hypertens. Res. 39:272-275.
(130) Lemaitre-Delaunay D., C. Pachiaudi, M. Laville, J. Pousin, M. Armstrong, and M. Lagarde. 1999. Blood compartmental metabolism of docosahexaenoic acid (DHA) in humans after ingestion of a single dose of [(13) C] DHA in phosphatidylcholine. J. Lipid Res. 40:1867-1874.
(131) Holub, B. J., M. Wlodek, W. Rowe, and J. Piekarski. 2009. Correlation of omega-3 levels in serum phospholipid from 2053 human blood samples with key fatty acid ratios. Nutr. J. 8:58.
(132) Mozaffarian, D., R. N. Lemaitre, I. B. King, X. Song, H. Huang, F. M. Sacks, E. B. Rimm, M. Wang, and D. S. Siscovick. 2013. Plasma phospholipid long-chain ω-3 fatty acids and total and cause-specific mortality in older adults: A cohort study. Ann. Intern. Med. 158:515-525.
(133) Middleton, P., J. C. Gomersall, J. F. Gould, E. Shepherd, S. F. Olsen, and M. Makrides. 2018. Omega-3 fatty acid addition during pregnancy. Cochrane Db. Syst. Rev. 2018: CD003402.
(134) Lee JW., Nishiumi S., Yoshida M., Fukusaki E., and Bamba T. 2013 Simultaneous profiling of polar lipids by supercritical fluid chromatography/tandem mass spectrometry with methylation. J. Chromatogr. A 1279:98-107.
(135) Morioka, S., Nakanishi, H., Yamamoto, T. et al. A mass spectrometric method for in-depth profiling of phosphoinositide regioisomers and their disease-associated regulation. Nat Commun 13, 83 (2022).
(136) Peng, L., Gawaz M., and Lämmerhofer M. 2021. Targeted Profiling of Short-, Medium-, and Long-Chain Fatty Acyl-Coenzyme As in Biological Samples by Phosphate Methylation Coupled to Liquid Chromatography-Tandem Mass Spectrometry. Anal. Chem. 93:9.

1. A method of generating a lipid profile using mass spectrometry (MS), the method comprising:
a) extracting a lipid fraction from a sample obtained from a subject;
b) protecting the amine of one or more lipids in the lipid fraction using an amine protecting reagent to provide one or more protected lipids;
c) methylating a phosphate ester to generate a phosphate methyl ester of the one or more protected lipids using 3-methyl-1-p-tolyltriazene (MTT) to generate one or more methylated lipids;
d) optionally back extracting the one or more methylated lipids;
e) optionally separating the one or more methylated lipids;
f) introducing the one or more methylated lipids to a mass spectrometer under positive-ion mode; and
g) acquiring a mass spectrum chart of the one or more methylated lipids to generate the lipid profile of the sample.

2. The method of claim 1, wherein the amine protecting reagent is 9-fluorenylmethyoxycarbonyl chloride (FMOC).

3. The method of claim 1, wherein separating comprises capillary electrophoresis, liquid chromatography or ion mobility.

4. The method of claim 1, wherein extracting the lipid fraction comprises incubating the sample with methyl tert butyl ether (MTBE), hexane, chloroform, methanol or acetonitrile.

5. The method of claim 1, wherein the one or more lipids is a phospholipid, glycerolipid, glycerophospholipid, sphingolipid, sterol, steroid, isoprenoid, glycolipid, polyketide, saccharolipid, prenol lipid, bile acid, fatty acid, a lipid containing a reactive amino, carboxyl, phenol, thiol, hydroxyl, or phosphate functionality (e.g., acylcarnitines, acyl-coenzyme A) or combinations thereof.

6. The method of claim 1, wherein the one or more lipids is a phospholipid optionally, the phospholipid is sphingomyelin (SM), phosphatidylcholine (PC), phosphatidylserine (PS), phosphatidylinositol (PI), phosphatidylethanolamine (PE), phosphatidic acid (PA), lysophosphatidylcholine (LPC), lysophosphatidylethanolamine (LPE), cardiolipin (CL) lysophosphatidic acid (LPA), or combinations thereof.

7. The method of claim 1, wherein:
(i) step c) has a reaction time of about 20 minutes to about 100 minutes, about 20 minutes to about 40 minutes, about 40 minutes to about 60 minutes, about 60 minutes to about 100 minutes, about 60 minutes to about 80 minutes, or about 40 minutes to about 80 minutes,
optionally the reaction time is about 60 minutes;
(ii) the concentration of MTT in step c) is about 50 mM to about 900 mM, optionally, the MTT is at a concentration of about 450 mM; and/or
(iii) step c) has a reaction temperature of about 20° C. to about 100° C., optionally the reaction temperature is about 60° C.

8. The method of claim 1, wherein:
(i) step b) has a reaction time of about 1 minute to about 30 minutes, optionally step b) has a reaction time of about 5 minutes; and/or
(ii) the concentration of the amine protecting reagent in step b) is about 0.1 mM to about 10 mM, optionally the concentration of the amine protecting reagent in step b) is about 0.85 mM.

9. The method of claim 1, wherein in step f) the mass spectrometer comprises multisegment injection-nonaqueous capillary electrophoresis-mass spectrometry (MSI-NACE-MS), direct infusion-MS, desorption ionization (DESI)-MS, gas chromatography (GC)-MS, ion mobility (IM)-MS liquid chromatography (LC)-MS or supercritical fluid chromatography (SFC)-MS (SFC)-MS/MS.

10. The method of claim 1, wherein the sample is of animal, plant or human origin, optionally the sample is from human blood, optionally plasma or serum.

11. The method of claim 1, wherein back extracting the one or more methylated lipids comprises back extracting with hexane.

12. A method of assessing omega-3 index (O3I) status in a subject, the method comprising:
a) obtaining a first sample from the subject at a first time point;
b) measuring the level of one or more omega-3 containing phospholipid biomarkers in the first sample; and
c) comparing the level of the one or more omega-3 containing phospholipid biomarkers in the first sample to a control level or value of the one or more omega-3 containing phospholipid biomarkers of known O3I status,
wherein similarity of level of the phospholipid biomarkers in the sample with the control indicates that the sample is from a subject with the known O3I status.

13. The method of claim 12, wherein the one or more omega-3 containing phospholipid biomarkers comprises one or more phosphatidylcholines (PCs) selected from the group consisting of PC 38:6 (16:0_22:6), PC 36:5 (16:0_20:5), PC 38:5, PC 40:6, PC 36:6, and PC 40:5, optionally the one or more PCs comprise one PC, two PCs, three PCs, four PCs, five PCs or six PCs.

14. The method of claim 13, wherein the one or more PCs comprise PC 36:5 (16:0_20:5) and PC 38:6 (16:0_22:6).

15. The method of claim 12, wherein the one or more omega-3 containing phospholipid biomarkers consist of two PCs, and wherein the two PCs consist of PC 36:5 (16:0_20:5) and PC 38:6 (16:0_22:6).

16. The method of claim 12 wherein the one or more omega-3 containing phospholipid biomarkers comprise PCs comprising omega-3 fatty acids containing eicosapentaenoic acid (EPA, 20:5). docosahexaenoic acid (DHA, 22:6), docosapentaenoic acid (DPA) and/or alpha-linolenic acid (ALA) together with other fatty acyl chains (e.g., 18:0).

17. The method of claim 12, wherein the sample comprises serum, plasma, whole blood or dried blood.

18. The method of claim 12, further comprising repeating the method of assessing 031 status for a second sample taken from the same subject at a second time point to assess O3I status and monitor change from the first time point to the second time point.

19. The method of claim 12, wherein assessing the level of one or more omega-3 containing phospholipid biomarkers comprises a method of chemical derivatization, the method comprising:
a) protecting the amine of the one or more omega-3 containing phospholipid biomarkers using an amine protecting reagent to provide one or more protected lipids, optionally the amine protecting reagent is FMOC;
b) methylating a phosphate ester of the one or more protected omega-3 containing phospholipid biomarkers using 3-methyl-1-p-tolyltriazene (MTT) to generate one or more methylated omega-3 containing phospholipid biomarkers; and
c) optionally introducing the one or more methylated lipids to a mass spectrometer under positive-ion mode and acquiring a mass spectrum chart of the one or more methylated lipids to generate the lipid profile of the sample.

20. A method of assessing cardiovascular risk in a subject, the method comprising: assessing omega-3 index (O3I) status in a subject using the method of claim 12, wherein if the level of the one or more omega-3 containing phospholipid biomarkers is similar to a control of less than 4% O3I the subject is determined to be at high cardiovascular risk, if the level of the one or more omega-3 containing phospholipid biomarkers is similar to a control of 4% to 8% O3I the subject is determined to be at intermediate cardiovascular risk, and if the level of the one or more omega-3 containing phospholipid biomarkers is similar to a control of more than 8% O3I the subject is determined to be at low cardiovascular risk.

21. The method of claim 20, wherein if the subject has a high or intermediate cardiovascular risk, the method further comprises treating the subject by administering omega-3 fatty acid supplementation, optionally the omega-3 fatty acid supplementation comprises fish oil, eicosapentaenoic acid (EPA) and/or docosahexaenoic acid (DHA).
